SEQUENCE LISTING (21 sequences)

SEQ ID NO: 1 - protein, 273 aa, Artificial Sequence
  Description of Artificial Sequence/Note = Synthetic Construct
STTSPTEETTQKLTVSHIEGYECQPIFLNVLEAIEPGVVCAGHDNNQPDSFAALLSSLNELGERQLVHVVKWAKALPGFRNLHVDDQMAVIQYSWMGLMVFAMGWRSFTNVNSRMLYFAPDLVFNEYRMHKSRMYSQCVRMRHLSQEFGWLQITPQEFLCMKALLLFSIIPVDGLKNQKFFDELRMNYIKELDRIIACKRKNPTSCSRRFYQLTKLLDSVQPIARELHQFTFDLLIKSHMVSVDFPEMMAEIISVQVPKILSGKVKPIYFHTQ

SEQ ID NO: 2 - protein, 344 aa, Artificial Sequence
  Description of Artificial Sequence/Note = Synthetic Construct
WSQPKTPVPAQRERAPVSGTQEKNKIRPRGQRDSSYYWEIEASEVMLSTRIGSGSFGTVYKGKWHGDVAVKILKVVDPTPEQFQAFRNEVAVLRKTRHVNILLFMGYMTKDNLAIVTQWCEGSSLYKHLHVQETKFQMFQLIDIARQTAQGMDYLHAKNIIHRDMKSNNIFLHEGLTVKIGDFGLATVKSRWSGSQQVEQPTGSVLWMAPEVIRMQDNNPFSFQSDVYSYGIVLYELMTGELPYSHINNRDQIIFMVGRGYASPDLSKLYKNCPKAMKRLVADCVKKVKEERPLFPQILSSIELLQHSLPKINRSASEPSLHRAAHTEDINACTLTTSPRLPVF

SEQ ID NO: 3 - protein, 20 aa, Artificial Sequence
  Description of Artificial Sequence/Note = Synthetic Construct
KMSKDGKKKKKKTKTKCIIM

SEQ ID NO: 4 - protein, 164 aa, Artificial Sequence [sequence omitted]

SEQ ID NO: 5 - protein, 495 aa, Artificial Sequence
  Description of Artificial Sequence/Note = Synthetic Construct
ATGTCAGAACCGGCTGGGGATGTCCGTCAGAACCCATGCGGCAGCAAGGCCTGCCGCCGCCTCTTCGGCCCAGTGGACAGCGAGCAGCTGAGCCGCGACTGTGATGCGCTAATGGCGGGCTGCATCCAGGAGGCCCGTGAGCGATGGAACTTCGACTTTGTCACCGAGACACCACTGGAGGGTGACTTCGCCTGGGAGCGTGTGCGGGGCCTTGGCCTGCCCAAGCTCTACCTTCCCACGGGGCCCCGGCGAGGCCGGGATGAGTTGGGAGGAGGCAGGCGGCCTGGCACCTCACCTGCTCTGCTGCAGGGGACAGCAGAGGAAGACCATGTGGACCTGTCACTGTCTTGTACCCTTGTGCCTCGCTCAGGGGAGCAGGCTGAAGGGTCCCCAGGTGGACCTGGAGACTCTCAGGGTCGAAAACGGCGGCAGACCAGCATGACAGATTTCTACCACTCCAAACGCCGGCTGATCTTCTCCAAGAGGAAGCCCTAA

SEQ ID NO: 6 - protein, 480 aa, Artificial Sequence
  Description of Artificial Sequence/Note = Synthetic Construct
MSDVAIVKEGWLHKRGEYIKTWRPRYFLLKNDGTFIGYKERPQDVDQREAPLNNFSVAQCQLMKTERPRPNTFIIRCLQWTTVIERTFHVETPEEREEWTTAIQTVADGLKKQEEEEMDFRSGSPSDNSGAEEMEVSLAKPKHRVTMNEFEYLKLLGKGTFGKVILVKEKATGRYYAMKILKKEVIVAKDEVAHTLTENRVLQNSRHPFLTALKYSFQTHDRLCFVMEYANGGELFFHLSRERVFSEDRARFYGAEIVSALDYLHSEKNVVYRDLKLENLMLDKDGHIKITDFGLCKEGIKDGATMKTFCGTPEYLAPEVLEDNDYGRAVDWWGLGVVMYEMMCGRLPFYNQDHEKLFELILMEEIRFPRTLGPEAKSLLSGLLKKDPKQRLGGGSEDAKEIMQHRFFAGIVWQHVYEKKLSPPFKPQVTSETDTRYFDEEFTAQMITITPPDQDDSMECVDSERRPHFPQFSYSASSTA

SEQ ID NO: 7 - DNA, 1443 bp, Artificial Sequence
  Description of Artificial Sequence/Note = Synthetic Construct
atgagcgacgtggctattgtgaaggagggttggctgcacaaacgaggggagtacatcaagacctggcggccacgctacttcctcctcaagaatgatggcaccttcattggctacaaggagcggccgcaggatgtggaccaacgtgaggctcccctcaacaacttctctgtggcgcagtgccagctgatgaagacggagcggccccggcccaacaccttcatcatccgctgcctgcagtggaccactgtcatcgaacgcaccttccatgtggagactcctgaggagcgggaggagtggacaaccgccatccagactgtggctgacggcctcaagaagcaggaggaggaggagatggacttccggtcgggctcacccagtgacaactcaggggctgaagagatggaggtgtccctggccaagcccaagcaccgcgtgaccatgaacgagtttgagtacctgaagctgctgggcaagggcactttcggcaaggtgatcctggtgaaggagaaggccacaggccgctactacgccatgaagatcctcaagaaggaagtcatcgtggccaaggacgaggtggcccacacactcaccgagaaccgcgtcctgcagaactccaggcaccccttcctcacagccctgaagtactctttccagacccacgaccgcctctgctttgtcatggagtacgccaacgggggcgagctgttcttccacctgtcccgggaacgtgtgttctccgaggaccgggcccgcttctatggcgctgagattgtgtcagccctggactacctgcactcggagaagaacgtggtgtaccgggacctcaagctggagaacctcatgctggacaaggacgggcacattaagatcacagacttcgggctgtgcaaggaggggatcaaggacggtgccaccatgaagaccttttgcggcacacctgagtacctggcccccgaggtgctggaggacaatgactacggccgtgcagtggactggtgggggctgggcgtggtcatgtacgagatgatgtgcggtcgcctgcccttctacaaccaggaccatgagaagctttttgagctcatcctcatggaggagatccgcttcccgcgcacgcttggtcccgaggccaagtccttgctttcagggctgctcaagaaggaccccaagcagaggcttggcgggggctccgaggacgccaaggagatcatgcagcatcgcttctttgccggtatcgtgtggcagcacgtgtacgagaagaagctcagcccacccttcaagccccaggtcacgtcggagactgacaccaggtattttgatgaggagttcacggcccagatgatcaccatcacaccacctgaccaagatgacagcatggagtgtgtggacagcgagcgcaggccccacttcccccagttctcctactcggccagcagcacggcctga

SEQ ID NO: 8 - protein, 98 aa, Artificial Sequence
  Description of Artificial Sequence/Note = Synthetic Construct
MHGDTPTLHEYMLDLQPETTDLYCYEQLNDSSEEEDEIDGPAGQAEPDRAHYNIVTFCCKCDSTLRLCVQSTHVDIRTLEDLLMGTLGIVCPICSQKP

SEQ ID NO: 9 - DNA, 294 bp, Artificial Sequence
  Description of Artificial Sequence/Note = Synthetic Construct
atgcatggagatacacctacattgcatgaatatatgttagatttgcaaccagagacaactgatctctactgttatgagcaattaaatgacagctcagaggaggaggatgaaatagatggtccagctggacaagcagaaccggacagagcccattacaatattgtaaccttttgttgcaagtgtgactctacgcttcggttgtgcgtacaaagcacacacgtagacattcgtactttggaagacctgttaatgggcacactaggaattgtgtgccccatctgttctcagaaacca

SEQ ID NO: 10 - DNA, 294 bp, Artificial Sequence
  Description of Artificial Sequence/Note = Synthetic Construct
atgcacggagatacacctacattgcatgaatatatgttagatttgcaaccagagacaactgatctctactgttatgagcaattaaatgacagctcagaggaggaggatgaaatagatggtccagctggacaagcagaaccggacagagcccattacaatattgtaaccttttgttgcaagtgtgactctacgcttcggttgtgcgtacaaagcacacacgtagacattcgtactttggaagacctgttaatgggcacactaggaattgtgtgccccatctgttctcagaaacca

SEQ ID NO: 11 - protein, 98 aa, Artificial Sequence
  Description of Artificial Sequence/Note = Synthetic Construct
MHGDTPTLHEYMLDLQPETTDLYCYEQLNDSSEEEDEVDGPAGQAEPDRAHYNIVTFCCKCDSTLRLCVQSTHVDIRTLEDLLMGTLGIVCPICSQKP

SEQ ID NO: 12 - DNA, 294 bp, Artificial Sequence
  Description of Artificial Sequence/Note = Synthetic Construct
atgcatggagatacacctacattgcatgaatatatgttagatttgcaaccagagacaactgatctctactgttatgagcaattaaatgacagctcagaggaggaggatgaagtagatggtccagctggacaagcagaaccggacagagcccattacaatattgtaaccttttgttgcaagtgtgactctacgcttcggttgtgcgtacaaagcacacacgtagacattcgtactttggaagacctgttaatgggcacactaggaattgtgtgccccatctgttctcagaaacca

SEQ ID NO: 13 - DNA, 294 bp, Artificial Sequence
  Description of Artificial Sequence/Note = Synthetic Construct
atgcacggagatacacctacattgcatgaatatatgttagatttgcaaccagagacaactgatctctactgttatgagcaattaaatgacagctcagaggaggaggatgaagtagatggtccagctggacaagcagaaccggacagagcccattacaatattgtaaccttttgttgcaagtgtgactctacgcttcggttgtgcgtacaaagcacacacgtagacattcgtactttggaagacctgttaatgggcacactaggaattgtgtgccccatctgttctcagaaacca

SEQ ID NO: 14 - protein, 648 aa, Artificial Sequence
  Description of Artificial Sequence/Note = Synthetic Construct
MEHIQGAWKTISNGFGLKDAVFDGSSCISPTIVQQFGYQRRASDDGKLTDSSKTSNTIRVFLPNKQRTVVNVRNGMSLHDCLMKALKVRGLQPECCAVFRLLQEHKGKKARLDWNTDAASLIGEELQVDFLDHVPLTTHNFARKTFLKLAFCDICQKFLLNGFRCQTCGYKFHEHCSTKVPTMCVDWSNIRQLLLFPNSTASDSGVPAPPSFTMRRMRESVSRMPASSQHRYSTPHAFTFNTSSPSSEGSLSQRQRSTSTPNVHMVSTTLPVDSRMIEDAIRSHSESASPSALSSSPNNLSPTGWSQPKTPVPAQRERAPGSGTQEKNKIRPRGQRDSSYYWEIEASEVMLSTRIGSGSFGTVYKGKWHGDVAVKILKVVDPTPEQLQAFRNEVAVLRKTRHVNILLFMGYMTKDNLAIVTQWCEGSSLYKHLHVQETKFQMFQLIDIARQTAQGMDYLHAKNIIHRDMKSNNIFLHEGLTVKIGDFGLATVKSRWSGSQQVEQPTGSVLWMAPEVIRMQDNNPFSFQSDVYSYGIVLYELMTGELPYSHINNRDQIIFMVGRGYASPDLSRLYKNCPKAMKRLVADCVKKVKEERPLFPQILSSIELLQHSLPKINRSASEPSLHRAAHTEDINACTLTTSPRLPVF

SEQ ID NO: 15 - DNA, 2977 bp, Artificial Sequence
  Description of Artificial Sequence/Note = Synthetic Construct
ccgaatgtgaccgcctcccgctccctcacccgccgcggggaggaggagcgggcgagaagctgccgccgaacgacaggacgttggggcggcctggctccctcaggtttaagaattgtttaagctgcatcaatggagcacatacagggagcttggaagacgatcagcaatggttttggattcaaagatgccgtgtttgatggctccagctgcatctctcctacaatagttcagcagtttggctatcagcgccgggcatcagatgatggcaaactcacagatccttctaagacaagcaacactatccgtgttttcttgccgaacaagcaaagaacagtggtcaatgtgcgaaatggaatgagcttgcatgactgccttatgaaagcactcaaggtgaggggcctgcaaccagagtgctgtgcagtgttcagacttctccacgaacacaaaggtaaaaaagcacgcttagattggaatactgatgctgcgtctttgattggagaagaacttcaagtagatttcctggatcatgttcccctcacaacacacaactttgctcggaagacgttcctgaagcttgccttctgtgacatctgtcagaaattcctgctcaatggatttcgatgtcagacttgtggctacaaatttcatgagcactgtagcaccaaagtacctactatgtgtgtggactggagtaacatcagacaactcttattgtttccaaattccactattggtgatagtggagtcccagcactaccttctttgactatgcgtcgtatgcgagagtctgtttccaggatgcctgttagttctcagcacagatattctacacctcacgccttcacctttaacacctccagtccctcatctgaaggttccctctcccagaggcagaggtcgacatccacacctaatgtccacatggtcagcaccacgctgcctgtggacagcaggatgattgaggatgcaattcgaagtcacagcgaatcagcctcaccttcagccctgtccagtagccccaacaatctgagcccaacaggctggtcacagccgaaaacccccgtgccagcacaaagagagcgggcaccagtatctgggacccaggagaaaaacaaaattaggcctcgtggacagagagattcaagctattattgggaaatagaagccagtgaagtgatgctgtccactcggattgggtcaggctcttttggaactgtttataagggtaaatggcacggagatgttgcagtaaagatcctaaaggttgtcgacccaaccccagagcaattccaggccttcaggaatgaggtggctgttctgcgcaaaacacggcatgtgaacattctgcttttcatggggtacatgacaaaggacaacctggcaattgtgacccagtggtgcgagggcagcagcctctacaaacacctgcatgtccaggagaccaagtttcagatgttccagctaattgacattgcccggcagacggctcagggaatggactatttgcatgcaaagaacatcatccatagagacatgaaatccaacaatatatttctccatgaaggcttaacagtgaaaattggagattttggtttggcaacagtaaagtcacgctggagtggttctcagcaggttgaacaacctactggctctgtcctctggatggccccagaggtgatccgaatgcaggataacaacccattcagtttccagtcggatgtctactcctatggcatcgtattgtatgaactgatgacgggggagcttccttattctcacatcaacaaccgagatcagatcatcttcatggtgggccgaggatatgcctccccagatcttagtaagctatataagaactgccccaaagcaatgaagaggctggtagctgactgtgtgaagaaagtaaaggaagagaggcctctttttccccagatcctgtcttccattgagctgctccaacactctctaccgaagatcaaccggagcgcttccgagccatccttgcatcgggcagcccacactgaggatatcaatgcttgcacgctgaccacgtccccgaggctgcctgtcttctagttgactttgcacctgtcttcaggctgccaggggaggaggagaagccagcaggcaccacttttctgctccctttctccagaggcagaacacatgttttcagagaagctctgctaaggaccttctagactgctcacagggccttaacttcatgttgccttcttttctatccctttgggccctgggagaaggaagccatttgcagtgctggtgtgtcctgctccctccccacattccccatgctcaaggcccagccttctgtagatgcgcaagtggatgttgatggtagtacaaaaagcaggggcccagccccagctgttggctacatgagtatttagaggaagtaaggtagcaggcagtccagccctgatgtggagacacatgggattttggaaatcagcttctggaggaatgcatgtcacaggcgggactttcttcagagagtggtgcagcgccagacattttgcacataaggcaccaaacagcccaggactgccgagactctggccgcccgaaggagcctgctttggtactatggaacttttcttaggggacacgtcctcctttcacagcttctaaggtgtccagtgcattgggatggttttccaggcaaggcactcggccaatccgcatctcagccctctcaggagcagtcttccatcatgctgaattttgtcttccaggagctgcccctatggggcgggccgcagggccagcctgtttctctaacaaacaaacaaacaaacagccttgtttctctagtcacatcatgtgtatacaaggaagccaggaatacaggttttcttgatgatttgggttttaattttgtttttattgcacctgacaaaatacagttatctgatggtccctcaattatgttattttaataaaataaattaaattt

SEQ ID NO: 16 - protein, 813 aa, Artificial Sequence
  Description of Artificial Sequence/Note = Synthetic Construct
MSRINFKKSSASTTPTSPHCPSPRLISLPRCASSSIDRKDQASPMASPSTPLYPKHSDSLHSLSGHHSAGGAGTSDKEPPKFKYKMIMVHLPFDQHSRVEVRPGETARDAISKLLKKRNITPQLCHVNASSDPKQESIELSLTMEEIASRLPGNELWVHSEYLNTVSSIKHAIVRRTFIPPKSCDVCNNPIWMMGFRCEFCQFKFHQRCSSFAPLYCDLLQSVPKNEDLVKELFGIASQVEGPDRSVAEIVLANLAPTSGQSPAATPDSSHPDLTSIKRTGGVKRHPMAVSPQNETSQLSPSGPYPRDRSSSAPNINAINDEATVQHNQRILDALEAQRLEEESRDKTGSLLSTQARHRPHFQSGHILSGARMNRLHPLVDCTPLGSNSPSSTCSSPPGGLIGQPTLGQSPNVSGSTTSSLVAAHLHTLPLTPPQSAPPQKISPGFFRNRSRSPGERLDAQRPRPPQKPHHEDWEILPNEFIIQYKVGSGSFGTVYRGEFFGTVAIKKLNVVDPTPSQMAAFKNEVAVLKKTRHLNVLLFMGWVREPEIAIITQWCEGSSLYRHIHVQEPRVEFEMGAIIDILKQVSLGMNYLHSKNIIHRDLKTNNIFLMDDMSTVKIGDFGLATVKTKWTVNGGQQQQQPTGSILWMAPEVIRMQDDNPYTPQSDVYSFGICMYEILSSHLPYSNINNRDQILFMVGRGYLRPDRSKIRHDTPKSMLKLYDNCIMFDRNERPVFGEVLERLRDIILPKLTRSQSAPNVLHLDSQYSVMDAVMRSQMLSWSYIPPATAKTPQSAAAAAARNKKAYYNVYGLI

SEQ ID NO: 17 - DNA, 1044 bp, Artificial Sequence
  Description of Artificial Sequence/Note = Synthetic Construct
atggagagagacttcgaccttggcatgggcagacctggcgggctcggaggacttggtggtgaaccgatcatgcaacaaatgccacagccagcgcctcatcatccatcccgtagtagtaacgaccacaatgtgaagaacctcatgaagcaggccgaggagaactccggatatctcacattgcaaggtaaccgtcgtaaagctgacttgaaggagcttcagttcgtggaagatattggtcatggaagctgcggtacggtcacaaagtgcagatacaagagtgtgatcatggctgtgaagacgatgcctcggacgtcaaacagttatgaaatgtcccgcattttgatggatcttgacgtcatctgtctctctttcgactgtccgtacattgtacgttgcttcggatacttcatcaccaacttcgacgtccgtgtctgcatggagtgcatggctacttgccttgaccgtctgcttatccgcatcaagcagccaattccagagagaattattggaaagttgagtgtgagcatcattaaagctctgcactacttgaaaaccaagcaccaaatcatgcaccgtgatgtgaagccatcaaacattctcctcgactggagtggcgtcatcaagctttgcgatttcggtattgctggcagactgatcgagtctcgtgctcattcgaagcaagccggatgccctctgtacatgggtcctgagcgcctcgaccccaacaactttgactcgtatgacattcgcagtgacgtgtggtcttttggtgttactttggtcgagctggcaaccggacagtacccatacgccggaaccgaattcgacatgatgtccaagattctcaatgacgagccaccgcgcctggatccggccaaattctctccggacttctgtcaactcgtcgagagctgcctgcagcgtgatccaacgatgcgtcccaattacgatatgctcctccagcatccgtttgtcgtgcatcacgagaaaattgaaaccgacgtcgaggagtggtttgccgatgtgatgggcgagtgcggataa

SEQ ID NO: 18 - protein, 1493 aa, Artificial Sequence
  Description of Artificial Sequence/Note = Synthetic Construct
MAAAAGDRASSSGFPGAAAASPEAGGGGGGGGALQGSGAPAAGAAGLLREPGSAGRERADWRRRQLRKVRSVELDQLPEQPLFLAAASPPCPSTSPSPEPADAAAGASRFQPAAGPPPPGAASRCGSHSAELAAARDSGARSPAGAEPPSAAAPSGREMENKETLKGLHKMEDRPEERMIREKLKATCMPAWKHEWLERRNRRGPVVVKPIPIKGDGSEVNNLAAEPQGEGQAGSAAPAPKGRRSPSPGSSPSGRSVKPESPGVRRKRVSPVPFQSGRITPPRRAPSPDGFSPYSPEETSRRVNKVMRARLYLLQQIGPNSFLIGGDSPDNKYRVFIGPQNCSCGRGAFCIHLLFVMLRVFQLEPSDPMLWRKTLKNFEVESLFQKYHSRRSSRIKAPSRNTIQKFVSRMSNSHTLSSSSTSTSSSENSIKDEEEQMCPICLLGMLDEESLTVCEDGCRNKLHHHCMSIWAEECRRNREPLICPLCRSKWRSHDFYSHELSSPVESPASLRAVQQPSSPQQPVAGSQRRNQESSFNLTHFGTQQIPSAYKDLAEPWIQVFGMELVGCLFSRNWNVREMALRRLSHDVSGALLLANGESTGNSGGGSGGSLSAGAASGSSQPSISGDVVEACCSVLSIVCADPVYKVYVAALKTLRAMLVYTPCHSLAERIKLQRLLRPVVDTILVKCADANSRTSQLSISTVLELCKGQAGELAVGREILKAGSIGVGGVDYVLSCILGNQAESNNWQELLGRLCLIDRLLLEFPAEFYPHIVSTDVSQAEPVEIRYKKLLSLLTFALQSIDNSHSMVGKLSRRIYLSSARMVTAVPAVFSKLVTMLNASGSTHFTRMRRRLMAIADEVEIAEVIQLGVEDTVDGHQDSLQAVAPTSCLENSSLEHTVHREKTGKGLSATRLSASSEDISDRLAGVSVGLPSSTTTEQPKPAVQTKGRPHSQCLNSSPLSHAQLMFPAPSAPCSSAPSVPDISKHRPQAFVPCKIPSASPQTQRKFSLQFQRNCSEHRDSDQLSPVFTQSRPPPSSNIHRPKPSRPVPGSTSKLGDATKSSMTLDLGSASRCDDSFGGGGNSGNAVIPSDETVFTPVEDKCRLDVNTELNSSIEDLLEASMPSSDTTVTFKSEVAVLSPEKAENDDTYKDDVNHNQKCKEKMEAEEEEALAIAMAMSASQDALPIVPQLQVENGEDIIIIQQDTPETLPGHTKAKQPYREDAEWLKGQQIGLGAFSSCYQAQDVGTGTLMAVKQVTYVRNTSSEQEEVVEALREEIRMMGHLNHPNIIRMLGATCEKSNYNLFIEWMAGGSVAHLLSKYGAFKESVVINYTEQLLRGLSYLHENQIIHRDVKGANLLIDSTGQRLRIADFGAAARLASKGTGAGEFQGQLLGTIAFMAPEVLRGQQYGRSCDVWSVGCAIIEMACAKPPWNAEKHSNHLALIFKIASATTAPSIPSHLSPGLRDVAVRCLELQPQDRPPSRELLKHPVFRTTW

SEQ ID NO: 19 - protein, 393 aa, Artificial Sequence [sequence omitted]

SEQ ID NO: 20 - protein, 393 aa, Artificial Sequence
  Description of Artificial Sequence/Note = Synthetic Construct
MPKKKPTPIQLNPAPDGSAVNGTSSAETNLEALQKKLEELELDEQQRKRLEAFLTQKQKVGELKDDDFEKISELGAGNGGVVFKVSHKPSGLVMARKLIHLEIKPAIRNQIIRELQVLHECNSPYIVGFYGAFYSDGEISICMEHMDGGSLDQVLKKAGRIPEQILGKVSIAVIKGLTYLREKHKIMHRDVKPSNILVNSRGEIKLCDFGVSGQLIDSMANSFVGTRSYMSPERLQGTHYSVQSDIWSMGLSLVEMAVGRYPIPPPDAKELELLFGCHVEGDAAETPPRPRTPGRPLSSYGMDSRPPMAIFELLDYIVNEPPPKLPSGVFSLEFQDFVNKCLIKNPAERADLKQLMVHAFIKRSDAEEVDFAGWLCSTIGLNQPSTPTHAASI

SEQ ID NO: 21 - protein, 393 aa, Artificial Sequence [sequence omitted]

(19) United States
(12) Patent Application Publication
(10) Pub. No.: US 2006/0088472 A1
(43) Pub. Date: Apr. 27, 2006

(54) E7 regulation of p21 cip1 through akt

(76) Inventors: Dennis J McCance, Rochester, NY (US); Thomas F Westbrook III, Rochester, NY (US)

Correspondence Address: NEEDLE & ROSENBERG, P.C., SUITE 1000, 999 PEACHTREE STREET, ATLANTA, GA, 30309-3915, US

(21) Appl. No.: 10/511,814

(22) PCT Filed: Apr. 21, 2003
(86) PCT No.: PCT/US03/12667

§ 371 Date: Sep. 12, 2005

Related U.S. Application Data
(60) Provisional application No. 60/374,245, filed on Apr. 19, 2002.

(51) Int. Cl. A61K 49/00; G01N 33/574

(52) U.S. Cl. 424/9.2; 435/7.23

(57) ABSTRACT

Disclosed are compositions and methods for inhibiting E7 effects on Akt, such as preventing the nuclear localization of p21^{Cip1}.

This application claims benefit of U.S. Provisional Application No. 60/374,245, filed May 19, 2002, which is hereby incorporated herein by reference in their entirety.

### BACKGROUND OF THE INVENTION

p21^{Cip1} is a potent inhibitor of cyclin-dependent kinase-2 (CDK2) activity. The human papillomavirus E7 oncoprotein abrogates p21^{Cip1}-mediated G1-arrest in response to anti-proliferative signals. The mechanism by which E7 antagonizes p21^{Cip1} function in vivo is unclear. Disclosed is the use of an engineered conditional Raf kinase that induces a p21^{Cip1}-mediated cell cycle arrest along with various other molecules.

Disclosed herein E7 abrogates Raf-associated arrest and prevents inhibition of cyclin E-CDK2 activity without disrupting Raf induction of p21^{Cip1}. E7 neither interacts with p21^{Cip1} nor derepresses p21^{Cip1}-associated CDK2 activity, but instead reduces the association between p21^{Cip1} and cyclin E-CDK2 complexes. Disclosed herein it is shown that Raf down-regulates steady-state levels of Akt, a regulator of p21^{Cip1} localization, leading to loss of p21^{Cip1} phosphorylation and accumulation of nuclear p21^{Cip1}. It is also shown herein that E7 disrupts the effects of Raf on Akt activity and prevents p21^{Cip1} nuclear accumulation. It is also diclosed herein that maintenance of Akt activity is necessary and sufficient to bypass Raf arrest.

### SUMMARY OF THE INVENTION

In accordance with the purposes of this invention, as embodied and broadly described herein, this invention, in one aspect, relates to compositions and methods for identifying inhibitors of E7 cell proliferation activity.

Additional advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate several embodiments of the invention and together with the description, serve to explain the principles of the invention.

FIG. 1 shows that E7 abrogates RafAR-induced arrest. (A) NIH3T3 cells stably expressing the RafAR fusion protein were infected with amphotrophic retroviruses expressing HPV-16 E7 (E7) or empty vector (Babe). Lysates were prepared and 100 μg of protein was resolved by 15% SDS-PAGE, transferred to nitrocellulose membrane, and probed with a monoclonal antibody directed against HPV-16 E7. (B and C) Pools of Babe- or E7-expressing cells growing in DMEM plus 10% NCS were treated with 0.02% ethanol (−) or 1.0 μM R1881 (+) for 30 hours and pulsed with BrdU for 30 minutes. Cells were trypsinized, fixed in 70% ethanol, and stained with propidium iodide (PI) for detection of total DNA content (B) and with α-BrdU-FITC for detection of DNA synthesis (C).

FIG. 2 shows that E7 prevents p21Cip1-mediated inhibition of cyclin E-CDK2 activity. (A) Expression levels of proteins involved in regulating the G1-S transition were examined during RafAR activation in the presence or absence of E7. Lysates were prepared from Babe- or E7-expressing cells treated with 0.02% ethanol (−) or 1.0 μM R1881 (+) for 30 hours, and 30 μg of protein was resolved by 12% SDS-PAGE, transferred to nitrocellulose membrane, and probed with antibodies to the indicated proteins. (B and C) Kinase activities of cyclin E, CDK2, and CDK4-associated complexes were assessed during RafAR activation. Lysates (50 μg) prepared as described in (A) were immunoprecipitated with the indicated antibodies, and immune-complexes were collected on Protein A-sepharose beads and assayed for kinase activity using histone H1 (for cyclin E-CDK2) and GST-RB c-terminus (for CDK4) as substrates (B). Quantitation of relative kinase activities in (B) is represented as a percentage of kinase activity from vehicle only (C).

FIG. 3 shows that p21^{Cip1} does not associate with E7 in C4 cells. Lysate was prepared from Babe-transduced cells treated with 1.0 μM R1881 for 30 hours. The indicated purified GST fusion proteins were incubated with lysate(A) or lysate supplemented with radiolabelled ³⁵S-E7 (B) for 12 hours at 4° C. Co-precipitated proteins were separated by 12% SDS-PAGE, transferred to nitrocellulose membrane, and the indicated proteins detected with the appropriate antibodies (A, both panels, B top panel) or by phosphorimager analysis (B, bottom panel). Input lanes represent 10% of extract or ³⁵S-E7 used per reaction.

FIG. 4 shows E7 does not derepress p21^{Cip1}-associated cyclin E-CDK2. (A) p21^{Cip1} is not associated with active cyclin E-CDK2 in the presence of E7. Whole cell lysates prepared from RafAR-induced E7-expressing cells were subjected to three rounds of immunodepletion with normal rabbit IgG or p21^{Cip1}-specific antibodies. Depleted lysates were analyzed by Western blotting with an antibody specific for p21^{Cip1} (top panel). Immunoprecipitations were performed on depleted lysates with a cyclin E-specific antibody and assayed for histone H1 kinase activity (bottom panel) as described in FIG. 2. (B) E7 does not render cyclin E-CDK2 resistant to p21^{Cip1}. Lysates (20 μg) from asynchronous Babe (▴) or E7 (♦) cells were mixed with increasing concentrations of purified recombinant GST-p21^{Cip1} and assayed for cyclin E-associated histone H1 kinase activity. Initial activity is represented as 100%.

FIG. 5 shows that enhanced cyclin E expression cannot overcome RafAR-induced arrest. (A) Cyclin E-CDK2 activity was not restored by exogenous cyclin E expression. NIH3T3 cells stably expressing the RafAR fusion protein were transduced with constructs expressing empty vector (Babe), HPV-16 E7 (E7), or human cyclin E (Cyclin E). Pools of infected cells were treated with 0.02% ethanol (−) or 1.0 μM R1881 (+) for 30 hours and assessed either for cyclin E-associated (top panels) or CDK2-associated (bottom panels) histone H1 kinase activity. An antibody specifically recognizing human cyclin E was utilized for immunoprecipitating cyclin E-associated kinase activity (top right panel) from cells expressing human cyclin E. (B) Exogenous cyclin E expression cannot abrogate RafAR-induced arrest. Cell lines were treated as in (A) and assessed for DNA synthesis via BrdU incorporation.

FIG. 6 shows that E7 alters p21^{Cip1}/cyclin E-CDK2 stoichiometry. (A) Lysates were prepared from Babe- or E7-expressing cells treated with 1.0 μM R1881 for 30 hours. The amount of lysate used was standardized to cyclin E steady state levels as determined by Western blotting (top panel). Cyclin E was immunoprecipitated and immune-complexes were analyzed for cyclin E-associated p21^{Cip1} by Western blotting with a p21^{Cip1}-specific antibody (bottom panel). (B) Lysates prepared from RafAR-induced Babe- or E7-expressing cells were subjected to three rounds of immunodepletion with normal rabbit IgG or p21^{Cip1}-specific antibodies. Depleted lysates were analyzed by Western blotting with antibodies specific for cyclin E (top panel), CDK2 (middle panel), or p21^{Cip1} (bottom panel).

FIG. 7 shows that E7 impairs RafAR-induced p21^{Cip1} nuclear accumulation. Babe- or E7-expressing cells were treated with 0.02% ethanol (−) or 1.0 μM R1881 for 30 hours, fixed with 3.7% paraformaldehyde, and stained with p21^{Cip1} specific antibody as described in Materials and Methods. Cells were scored for nuclear or whole-cell localization of p21^{Cip1}. (A) Representative fluorescent (left panels) and phase contrast (right panels) micrographs of RafAR-induced Babe- (upper panels) or E7-expressing cells (lower panels) are shown. (B) Quantitation of cells exhibiting nuclear localization of p21^{Cip1}. The number of cells with nuclear p21^{Cip1} accumulation is represented as a percentage of total cells counted. The average and deviation values shown are from two independent experiments with at least 200 cells counted per experiment.

FIG. 8 shows that the P13-K/Akt pathway is involved in E7-mediated abrogation of RafAR-induced arrest. (A) E7-expressing cells were treated for 30 hours with 1.0 μM R1881 in the absence or presence of increasing concentrations of LY294002 (50 μM, 100 μM, 200 μM), an inhibitor of PI-3K activity. DNA synthesis was measured via BrdU incorporation. (B) E7-expressing cells were treated with 1.0 uM R1881 in the presence or absence of 100 uM LY294002, stained with p21^{Cip}1-specific antibody, and quantitated for nuclear p21^{Cip1} localization as in FIG. 7. (C) Babe- or E7-expressing cells were co-transfected with a GFP-expression plasmid (400 ng) and the indicated plasmids (3.2 μg). After transfection, cells were treated with 0.02% ethanol or 1.0 mM R1881 for 30 hours, with BrdU added for the last 10 hours of treatment. Cells were stained with α-BrdU, and GFP-positive cells were scored for BrdU incorporation via indirect immunofluorescence. Percentages of BrdU incorporation were calculated by defining the number obtained from vehicle-treated cells as 100%. The average and deviation values shown are from three independent experiments with at least 150 GFP-positive cells counted per experiment. Low deviation values for some samples could not be resolved in this figure. (D) Lysates were prepared from Babe- or E7-expressing cells treated with 0.02% ethanol (−) or 1.0 μM R1881 (+) for 30 hours and analyzed by Western blotting with antibodies directed against total Akt (top panel) or Akt phosphorylated on serine 473 (P-Akt), representing the active form of Akt. (E) Lysates prepared from Babe- or E7-expressing cells treated as in (D) were subjected to immunoprecipitation with an antibody specific for p21^{Cip1}. Precipitated p21^{Cip1} was analyzed for threonine-phosphorylation by Western blotting with a phospho-threonine-specific antibody (top panel). p21^{Cip1}-expression was analyzed in lysates used for above immunoprecipitations by Western blotting with a p21^{Cip1}-specific antibody (bottom panel).

FIG. 9 shows that the LXCXE motif of E7 is necessary to prevent p21^{Cip1}-mediated inhibition of cyclin E-CDK2. (A) NIH3T3 cells stably expressing the RafAR fusion protein were transduced with constructs expressing empty vector (Babe), E7, or E7.C24G. Lysates were prepared and examined for E7 expression with a monoclonal antibody directed against HPV-16 E7. (B) Pools of infected cells were treated with 0.02% ethanol (−) or 1.0 μM R1881 (+) for 30 hours and assessed for DNA synthesis via BrdU incorporation. Low deviation values for some samples could not be resolved in this figure. (C) Babe-, E7-, or E7.C24G-expressing cells were treated as in (A) and assessed for cyclin E-associated histone H1 kinase activity (top panel) or cellular levels of active Akt by Western blotting with an antibody specifically recognizing Akt phosphorylated on serine 473 (P-Akt, bottom panel).

FIG. 10 shows inhibition of P13-K activity restores p21^{Cip1} nuclear localization in E7-expressing cells. E7-expressing cells were treated for 30 hours with 1.0 μM R1881 in the absence or presence of 100 μM LY294002, an inhibitor of PI-3K activity. Cells were fixed with 3.7% paraformaldehyde, and stained with p21^{Cip1}-specific antibody as described in Materials and Methods. Cells were scored for nuclear or whole-cell localization of p21^{Cip1}. The number of cells with nuclear p121^{Cip1} accumulation is represented as a percentage of total cells counted. The average and deviation values shown are from two independent experiments with at least 200 cells counted per experiment.

FIG. 11 shows p21^{Cip1}-associated cyclin E-CDK2 is inactive in E7-expressing human keratinocytes. Whole cell lysates prepared from E7-expressing human keratinocytes were subjected to three rounds of immunodepletion with normal rabbit IgG or p21^{Cip1}-specific antibodies. Depleted lysates were analyzed by Western blotting with an antibody specific for p21^{Cip1} (top panel). Immunoprecipitations were performed on depleted lysates with cyclin E-specific (middle panel) or CDK2-specific (bottom panel) antibodies and assayed for histone H1 kinase activity.

FIG. 12 shows that E7 Reduces p27^{Kip1} nuclear accumulation. (A) Control or E7-expressing NIH3T3 cells were grown to confluence and subsequently subjected to serum-starvation (0.5% BCS) for 24 hours. Cells were collected and analyzed by Western blot with p27^{Kip1}-specific antibody. (B) Confluent, serum-starved cells were fixed with 3.7% paraformaldehyde, and stained with p27^{Kip1}-specific antibody as described in Materials and Methods. Images shown are 40× magnification.

FIG. 13 shows a blot for active Akt (P-Akt) and total Akt (Akt) after treatment of NIH3T3 cells with R1881.

FIG. 14 shows that Raf inactivates active Akt by causing the dephosphorylation of Akt at the earelier time points. The drugs and R1881 were added at 0 hr and the samples were taken at 6 hrs post-treatment.

FIG. 15 shows that E7 diminishes TGF-β-induced p27^{Kip1} nuclear localization. (A) Tet-E7 Mv1Lu cells were treated for 24 hours with 2 μg/mL doxycycline or vehicle. Cell lysates were analyzed by Western blotting with antibody specific for HPV16 E7. (B) Tet-E7 Mv1Lu cells were treated for 24 hours with 3 ng/mL TGF-β in the presence or absence of 2 μg/mL doxycycline and subsequently analyzed for p27^{Kip1} expression via Western blot. (C, D) Tet-E7 Mv1Lu cells were treated as in (B), fixed with 3.7% paraformaldehyde, and stained with p27^{Kip1} specific antibody as described in Materials and Methods. (C) Representative fluorescent (left panels) and phase contrast (right panels) micrographs of TGF-β treated cells in the absence (upper panels) or presence (lower panels) of doxycycline. (D) Quantitation of cells exhibiting nuclear localization of p27^{Kip1}. The number of cells with nuclear p27^{Kip1} accumulation is represented as a percentage of total cells counted. The average and deviation values shown are from two independent experiments with at least 200 cells counted per experiment.

FIG. 16 A shows a plot of the % BrdU incorporation as a function of adriamycin addition in control cells and E7 cells. FIG. 16B shows the % incorporated of P21Cip1 in control and E7 cells. FIG. 16 shows the presence of the active form of Akt in the presence or absence of adriamycin and in control cells or E7 cells.

### DETAILED DESCRIPTION

The present invention may be understood more readily by reference to the following detailed description of preferred embodiments of the invention and the Examples included therein and to the Figures and their previous and following description.

Before the present compounds, compositions, articles, devices, and/or methods are disclosed and described, it is to be understood that this invention is not limited to specific synthetic methods, specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint: It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

Throughout this application, various publications are referenced. The disclosures of these publications in their entireties are hereby incorporated by reference into this application in order to more fully describe the state of the art to which this invention pertains. The references disclosed are also individually and specifically incorporated by reference herein for the material contained in them that is discussed in the sentence in which the reference is relied upon.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

### A. COMPOSITIONS AND METHODS

Disclosed herein E7 promotes oncogenesis through an inhibition of p21^{Cip1} transport into the nucleus. p21^{Cip1} acts as a cell cycle regulator after transport into the nucleus by binding CDK2/cyclin complexes, such that the cell cycle progression signals of the CDK2/cyclin complex are blocked. Akt is a kinase, which phosphorylates p21^{Cip1} in the nuclear localization signal (NLS) of p21^{Cip1}. Upon phosphorylation nuclear transport does not occur and p21^{Cip1} is ubiquinated and subsequently degraded. Disclosed herein, Raf induces cell cycle arrest through increasing the level of p21^{Cip1} in a cell and in the nucleus. Disclosed herein, Raf accomplishes this by increasing the expression of p21^{l Cip1} as well as promoting the degradation of Akt, the negative regulator of p21^{Cip1}. The HPV protein, E7 abbrogates the G1 cell cycle arrest. Disclosed herein, E7 accomplishes this abrogation not by decreasing the expression of p21^{Cip1}, but rather by preventing the degradation of Akt. Thus, E7 is an Akt activity promoting molecule. Furthermore, E7 is an Akt maintaining molecule. This effect of E7 on Akt allows for the identifification and production of molecules that inhibit E7 oncogenic effects on a cell.

The cellular response to oncogenic Ras depends upon the presence or absence of cooperating mutations. In the absence of immortalizing oncogenes or genetic lesions, activation of the Ras/Raf pathway results in a p21^{Cip1}-dependent cellular arrest. The human papillomavirus oncoprotein E7 transforms primary cells in cooperation with Ras and abrogates p21^{Cip1}-mediated growth arrest in the presence of various antimitogenic signals. Disclosed is a conditional Raf molecule to investigate the effects of E7 on p21^{Cip1} function in the context of Raf-induced cellular arrest. E7 bypassed Raf-induced arrest and alleviated inhibition of cyclin E-CDK2 without suppressing Raf-specific synthesis of p21^{Cip1} or derepressing p21^{Cip1}-associated CDK2 complexes. Activation of Raf led to nuclear accumulation of p21^{Cip1}, and provided herein is evidence that this effect is mediated by inhibition of Akt, a regulator of p21^{Cip1} localization. Disclosed herein, loss of Akt activity is an important event in the cellular arrest associated with Raf-induction, since maintenance of Akt activity was necessary and sufficient to bypass Raf-induced arrest. In agreement, expression of E7 sustained Akt activity and reduced nuclear accumulation of p21^{Cip1}, resulting in decreased association between p21^{Cip1} and cyclin E-CDK2. Disclosed herein, E7 inhibits p21^{Cip1} function in the context of Raf signaling by altering Raf-Akt antagonism and preventing the proper subcellular localization of p21^{Cip1}.

Disclosed herein E7 can maintain Akt activity when cells are given an arrest signal, such as that caused by cRaf-1, and that this results in the cells being able to bypass the arrest. One of the arrest signals used was the over-expression of cRaf-1. Disclosed herein cRaf-1 can inactivate Akt. Also disclosed E7 can abrogate this inhibition and so maintain Akt activity. Disclosed herein the ability of HPV-16 E7 to bypass the arrest is dependent on the capacity of E7 to bind and inactivate Rb. This is consistent with E7 affecting the transcriptional regulation of a gene(s) that control Akt activity.

Disclosed herein the inactivation of Akt occurs within 3 to 6 hours after induction of cRaf-1. Cell cycle arrest is not evident until 16 to 20 hours after induction of cRaf-1. Also disclosed, the Akt proteins levels do not change at the early time points, but the active, phosphorylated form of Akt disappears at the early time points. It is also shown herein that cRaf-1 inhibits Akt activity through MEK1 (MAPkinasekinase1), which is a downstream target of cRaf-1, using MEK1 inhibitors. Furthermore, new protein synthesis is required for the inhibition of Akt, by the cell cycle arrestor. This was shown using cyclohexamide, which interferes with protein translation. It is also shown herein that degradation of activate Akt through the proteosome pathway does not occur, and this was shown through using proteosome inhibitors. Also disclosed herein the upstream effector of Akt, namely PI3 kinase activity, is not affected by the cRaf-1. Overall these results indicate that the inactivation of Akt is through a phosphatase enzyme that removes the activating phosphate group on Akt. The data is consistent with E7 controlling the activity of the phosphatase.

Systems designed to enhance this E7 effect are disclosed and these systems can be used for identifying molecules that modulate this effect. Thus, disclosed are compositions and methods related to identifying molecules that affect E7 related events in cells. Also disclosed are compoisitions that inhibit the E7 maintenance of Akt activity. Also disclosed herein E7 does not need to alter the amount of Akt gene expression to modulate the amount of Akt in the cell.

### B. COMPOSITIONS

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular E7 is disclosed and discussed and a number of modifications that can be made to a number of molecules including the E7 are discussed, specifically contemplated is each and every combination and permutation of E7 and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

The disclosed compositions and methods are involved in the modulation of the cell cycle. The disclosed compositions and methods utilize the information disclosed herein that E7 regulates p21^{Cip1} activity by preventing p21^{Cip1} import into the nucleus through E7 regulation of Akt, a regulator of p21^{Cip1}. Disclosed herein, E7 is shown to regulate Akt by down regulating Akt degradation.

### 1. Molecules Involved in the Cell Cycle

Regulation of mammalian cell proliferation is governed by signaling pathways that ultimately converge on the activity of cyclin-dependent kinases (CDKs). Progression through the G1 phase of the mitotic cell cycle is controlled by the activities of the G1 CDKs, cyclin D-CDK4/6 and cyclin E-CDK2 (Sherr, 1994a; Sherr, 1996). Cyclins D and E are rate-limiting for progression through G1, as both cyclins shorten G1 when overexpressed and are essential for G1-S transition in higher eukaryotes (Ohtsubo et al., 1995; Quelle et al., 1993). An important function of the G1 CDKs is to phosphorylate and inactivate the retinoblastoma tumor suppressor protein (RB) (Sherr, 1994b; Weinberg, 1995). RB is a negative regulator of cell proliferation that exerts its effects, at least in part, by associating with various transcription factors (in particular, members of the E2F/DP family) and repressing transcription (Weinberg, 1995). The targets of RB-mediated regulation include genes required for G1-S progression (E2F1, cyclin E, cyclin A) and DNA synthesis (DNA polα, DHFR, thymidine synthetase), underlining the central role of RB in governing cell proliferation (Weinberg, 1995). In addition, cyclin E- and A-CDK2 complexes have essential non-RB substrates, as attenuation of CDK2 arrests cells in G1 in the absence of a functional RB pathway (Alevizopoulos et al., 1997; Hofmann and Livingston, 1996; Lukas et al., 1997).

a) Cyclin Dependent Kinase Inhibitors

The activity of CDKs is regulated at multiple tiers including association with cyclins, nuclear localization, activating and inactivating phosphorylations, and association with specific CDK-inhibitors (CKIs) (Morgan, 1995). The physiologic importance of CKIs is emphasized by the observation that many antiproliferative signals lead to increased expression of these inhibitory molecules and that cells deficient for expression of specific CKIs demonstrate defects in cell cycle control (Harper and Elledge, 1996; Sherr and Roberts, 1995). Mammalian CKIs are divided into two classes based on primary sequence homology. The INK4 family of CKIs (p16^{Ink4a}, p15^{Ink4b}, p18^{Ink4c}, p19^{Ink4d}) interacts with and inhibits CDK4/6 by preventing their association with D-type cyclins. CKIs of the Kip/Cip family (p21^{Cip1}, p27^{Kip1}, p57^{Kip2}) inhibit a broader spectrum of CDKs.

b) p21^{Cip1}

P21^{Cip1} associates with complexes of cyclins D1/D2/D3-CDK4/6 and cyclins E/A-CDK2 (Harper et al., 1993; Harper et al., 1995). The N-terminus of p21^{Cip1} is conserved among Kip/Cip CKIs and contains cyclin- and CDK-binding sites that are necessary for inhibiting CDK activity and cell cycle arrest (Sherr and Roberts, 1995). The C-terminal half of p21^{Cip1} contains a second cyclin-binding motif, a PCNA-binding domain, and a nuclear localization sequence (NLS). p21^{Cip1} is a potent inhibitor of CDK2 in vivo. Indeed, CDK2 represents an important target of p21^{Cip1} in regulating proliferation, since numerous antimitogenic stimuli, including growth factor starvation, contact-inhibition, anchorage detachment, DNA damage, and TGF-β, result in Kip/Cip-mediated inhibition of cyclin E-CDK2 and subsequent cell cycle arrest (Coats et al., 1996; Dulic et al., 1994; Fang et al., 1996; Koff et al., 1993; Polyak et al., 1994a; Polyak et al., 1994b). Transcription of p21^{Cip1} is activated by the p53 tumor suppressor in response to DNA damage, and p21^{Cip1} is an essential determinant of p53-induced G1 arrest (el-Deiry et al., 1993). In agreement, p21Cip1−/− embyronic fibroblasts do not undergo G1 arrest following genotoxic stress or nucleotide perturbation (Brugarolas et al., 1995; Deng et al., 1995). p21^{Cip1} has also been implicated in p53-independent senescence-derived arrest and in terminal differentiation of myoblast, epithelial, and hematopoietic cell lineages (Missero et al., 1995; Missero et al., 1996; Noda et al., 1994; Parker et al., 1995). In addition, it has been suggested that p21^{Cip1} is an important target in the oncogenic activity of HER-2/neu and Akt, since phosphorylation of the p21^{Cip1} NLS by Akt, a downstream effector of the PI3-K signaling pathway, has recently been shown to alter the subcellular localization and cell-growth inhibitory function of p21^{Cip1} (Zhou et al., 2001).

### 2. Human Papillomaviruses (HPV)

Human papillomaviruses (HPV) are small DNA viruses that require unscheduled S phase entry in terminally differentiated epithelial keratinocytes in order for viral genome amplification to occur (Laimins, 1993). Not surprisingly, HPV have evolved several strategies for uncoupling differentiation from cell cycle arrest. The E7 early gene product of HPV16 stimulates cellular progression through the G I -S transition in the presence of various G1-arrest signals and can immortalize and transform several cell types alone or in cooperation with activated ras (Banks et al., 1990; Edmonds and Vousden, 1989; Phelps et al., 1992), suggesting E7 has evolved to interact with key components of cellular growth-regulatory pathways.

E7 has been shown to target the RB family of pocket proteins (RB, p 107, p 130) (Halpern, 1997). E7 interacts with the pocket proteins through an LXCXE motif and can disrupt RB-mediated gene regulation. In addition, expression of E7 leads to a reduction in the steady state level of RB by ubiquitin-dependent degradation (Boyer et al., 1996). E7 also abrogates G1 arrest induced by DNA damage, epithelial differentiation, TGF-β, growth factor withdrawal, and anchorage detachment (Banks et al., 1990; Demers et al., 1996; Funk et al., 1997; Hickman et al., 1997; Jones and Munger, 1997; Ruesch and Laimins, 1998; Schulze et al., 1998), stimuli that negatively regulate proliferation via p21^{Cip1} or the closely related p27^{Kip1}.

Premalignant lesions caused by HPV occur in all stratified squamous epithelia and E7 is essential for the generation of new infectious virus and the abnormalities observed in the infected epithelium. E7 induces AKT activity, which over-rides the inhibitory activity of p21CIP. There are other cancers, such as breast cancer, where there is an increase in the kinase inhibitor, p27KIP (related to p21CIP). There is also a relationship between the high level of p27KIP and poor prognosis for the patient. The cellular localization of the p27KIP in these cancers is similar to that of E7-expressing cells and suggests that there is a celluar function, which is altered in these breast cancer cells which is also altered in E7-expressing cells.

### 3. Systems Which can be Used to Identify Modulators of E7 Activity

Disclosed are systems which can be used to identify compounds that affect the E7 maintenance of Akt in a cell. There a number of components which are present in these systems. It is understandable that the components are general and that they may be substituted with functional equivalents. One aspect of the systems is that the systems should be able to up-regulate p21^{Cip1} production. One way of doing this is to have a regulatable Raf gene present in the system, expressed from, for example, a vector encoding a Raf gene under the control of a regulatable promoter. A system incorporating Raf in this manner allows for contollable upregulation of p21^{Cip1} production in the system in a physiological manner.

The systems, also will typically express Akt, or some form of Akt. It is understood that there are active forms of Akt (phosphorylated) and in active forms of Akt (unphosphorylated).

The system also typically will include a means of expressing E7, such as vector encoding E7. This aspect of the system allows for the E7 maintenance of Akt to be observed. The expression of E7 can either be regulatable or constitutive.

The system then can comprise a variety of components, such as potential inihibtors of E7 or PI3K or active Akt. The system could, for example, comprise a dominant-negative mutant of Akt, such as Akt K179M. This type of system can be used as a control, for example, to compare the inihibtor effects of potential Akt inihibitors, by for example, comparing the potential inhibitors activity to that of the inhibitor Akt K179M, which is disclosed herein. The system can also further include a means for providing E7 point mutants, such as vectors expressing E7 point mutations. For example, the E7 point mutation may be the E7.C24G point mutation, which is a mutation that prevents interaction with Rb. The point mutation has reduced ability to bind Akt. This type of system can also be used as a control, for comparing the effect of other inhibitors of Akt maintenance activity. Typically, only E7 is required. Also, typically only the enzymatic domain of Raf is required along with a localization signal for obtaining appropriate Raf function within the systems. [Sewing et al. 1997 Mol. Cell Biol. 17: 5588-5597].

Also disclosed are systems which have a regulatable Raf component as well as a component that produces stable expression of human cyclin E.

All of the various components can be expressed in a variety of ways. For example, they can be constitutively expressed as described herein, or regulatable. Furthermore, they can be expressed through activation of endogenous genes, for example in cell types which may not normally express one or more of the proteins, but which may have a beneficial background for looking at the effects of E7. It is also understood herein that functional variants and alleles exist for all of the proteins making up the disclosed systems.

These systems can be utilized in a variety of cells, such as NIH3T3. Other cell types such as human foreskin keratinocytes (HFK) can be used when arrested through DNA damage. Also NIH3T3 cells arrested by serum starvation can be used. In this latter case p27^{KIP} is the cyclin kinase inhibitor which is bypassed. One of the cell types that can be used is epithelial cells. For example mink lung cells, such as Mv1Lu, can be used. Typically the cells used must be capable of arrest using a pathway that involves Akt. For example any type of cell, immortalized with Raf or Raf component, such as cRaf-1, will have these properties.

The systems will typically contain or be under some type of arrest signal. For example, the cells can be arrested by Raf or a Raf variant, or cRaf-1. The cells can also be arrested through for example, DNA damage, such as that caused by Adriamycin (see Example 3). The cells can also be arrested by TGF-β or by serum starvation. Cells can also be arrested by differentiation, for example human keratinocytes (HFK).

The systems are designed such that various activities or components or states, for example, can be assayed. These compnonents, activities, or states, for example, are related as disclosed herein to the abrogation by E7 of the arrest of the cell cycle through the effects on Akt. For example, the amount of active Akt or total Akt, can be measured in the systems as an indication of the effect E7 is having on the system, as when cells are arrested as disclosed herein, there is ultimate decrease in active Akt amount. Furthermore, the amount of active Akt, phosphprylated, can be measured, by for example, probing with antibodies to the phosphorylated form. Furthermore, the activity of Akt can be measured, by for example, looking at P21^{cip} presence. The systems can also look at a variety of factors, such as MEK-1 activity or P21Cip activity, or cyclin accumulation or CDK-cyclin complex formation or Rb activity, or E7-Rb complex formation, which are related to the arrest of cells and the abrogation of this arrest by E7.

It is understood that the disclosed methods and systems for identifying molecules that inhibit E7 abbrogation, for example, can be performed using high through put means. For example, putative inhibitors can be identified using Fluorescence Resonance Energy Transfer (FRET) to quickly identify interactions. The underlying theory of the techniques is that when two molecules are close in space, ie, interacting at a level beyond background, a signal is produced or a signal can be quenched. Then, a variety of experiments can be performed, including, for example, adding in a putative inhibitor. If the inhibitor competes with the interaction between the two signaling molecules, the signals will be removed from each other in space, and this will cause a decrease or an increase in the signal, depending on the type of signal used. This decreasing or increasing signal can be correlated to the presence or absence of the putative inhibitor. Any signaling means can be used. For example, disclosed are methods of identifying an inhibitor of the interaction between any two of the disclosed molecules comprising, contacting a first molecule and a second molecule together in the presence of a putative inhibitor, wherein the first molecule or second molecule comprises a fluorescence donor, wherein the first or second molecule, typically the molecule not comprising the donor, comprises a fluorescence acceptor; and measuring Fluorescence Resonance Energy Transfer

In general, it is understood that one way to define any known variants and derivatives or those that might arise, of the disclosed genes and proteins herein, is through defining the variants and derivatives in terms of homology to specific known sequences. This identity of particular sequences disclosed herein is also discussed elsewhere herein. In general, variants of genes and proteins herein disclosed typically have at least, about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent homology to the stated sequence or the native sequence. Those of skill in the art readily understand how to determine the homology of two proteins or nucleic acids, such as genes. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment. It is understood that any of the methods typically can be used and that in certain instances the results of these various methods may differ, but the skilled artisan understands if identity is found with at least one of these methods, the sequences would be said to have the stated identity, and be disclosed herein.

For example, as used herein, a sequence recited as having a particular percent homology to another sequence refers to sequences that have the recited homology as calculated by any one or more of the calculation methods described above. For example, a first sequence has 80 percent homology, as defined herein, to a second sequence if the first sequence is calculated to have 80 percent homology to the second sequence using the Zuker calculation method even if the first sequence does not have 80 percent homology to the second sequence as calculated by any of the other calculation methods. As another example, a first sequence has 80 percent homology, as defined herein, to a second sequence if the first sequence is calculated to have 80 percent homology to the second sequence using both the Zuker calculation method and the Pearson and Lipman calculation method even if the first sequence does not have 80 percent homology to the second sequence as calculated by the Smith and Waterman calculation method, the Needleman and Wunsch calculation method, the Jaeger calculation methods, or any of the other calculation methods. As yet another example, a first sequence has 80 percent homology, as defined herein, to a second sequence if the first sequence is calculated to have 80 percent homology to the second sequence using each of calculation methods (although, in practice, the different calculation methods will often result in different calculated homology percentages).

### 5. Hybridization/Selective Hybridization

The term hybridization typically means a sequence driven interaction between at least two nucleic acid molecules, such as a primer or a probe and a gene. Sequence driven interaction means an interaction that occurs between two nucleotides or nucleotide analogs or nucleotide derivatives in a nucleotide specific manner. For example, G interacting with C or A interacting with T are sequence driven interactions. Typically sequence driven interactions occur on the Watson-Crick face or Hoogsteen face of the nucleotide. The hybridization of two nucleic acids is affected by a number of conditions and parameters known to those of skill in the art. For example, the salt concentrations, pH, and temperature of the reaction all affect whether two nucleic acid molecules will hybridize.

Parameters for selective hybridization between two nucleic acid molecules are well known to those of skill in the art. For example, in some embodiments selective hybridization conditions can be defined as stringent hybridization conditions. For example, stringency of hybridization is controlled by both temperature and salt concentration of either or both of the hybridization and washing steps. For example, the conditions of hybridization to achieve selective hybridization may involve hybridization in high ionic strength solution (6×SSC or 6×SSPE) at a temperature that is about 12-25° C. below the Tm (the melting temperature at which half of the molecules dissociate from their hybridization partners) followed by washing at a combination of temperature and salt concentration chosen so that the washing temperature is about 5° C. to 20° C. below the Tm. The temperature and salt conditions are readily determined empirically in preliminary experiments in which samples of reference DNA immobilized on filters are hybridized to a labeled nucleic acid of interest and then washed under conditions of different stringencies. Hybridization temperatures are typically higher for DNA-RNA and RNA-RNA hybridizations. The conditions can be used as described above to achieve stringency, or as is known in the art. (Sambrook et al., Molecular Cloning: A Laboratory Manual, 2nd Ed., Cold Spring Harbor Laboratory, Cold Spring Harbor, New York, 1989; Kunkel et al. Methods Enzymol. 1987:154:367, 1987 which is herein incorporated by reference for material at least related to hybridization of nucleic acids). A preferable stringent hybridization condition for a DNA:DNA hybridization can be at about 68° C. (in aqueous solution) in 6×SSC or 6×SSPE followed by washing at 68° C. Stringency of hybridization and washing, if desired, can be reduced accordingly as the degree of complementarity desired is decreased, and further, depending upon the G-C or A-T richness of any area wherein variability is searched for. Likewise, stringency of hybridization and washing, if desired, can be increased accordingly as homology desired is increased, and further, depending upon the G-C or A-T richness of any area wherein high homology is desired, all as known in the art.

Another way to define selective hybridization is by looking at the amount (percentage) of one of the nucleic acids bound to the other nucleic acid. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the limiting nucleic acid is bound to the non-limiting nucleic acid. Typically, the non-limiting primer is in for example, 10 or 100 or 1000 fold excess. This type of assay can be performed at under conditions where both the limiting and non-limiting primer are for example, 10 fold or 100 fold or 1000 fold below their k_{d}, or where only one of the nucleic acid molecules is 10 fold or 100 fold or 1000 fold or where one or both nucleic acid molecules are above their k_{d}.

Another way to define selective hybridization is by looking at the percentage of primer that gets enzymatically manipulated under conditions where hybridization is required to promote the desired enzymatic manipulation. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer is enzymatically manipulated under conditions which promote the enzymatic manipulation, for example if the enzymatic manipulation is DNA extension, then selective hybridization conditions would be when at least about 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer molecules are extended. Preferred conditions also include those suggested by the manufacturer or indicated in the art as being appropriate for the enzyme performing the manipulation.

Just as with homology, it is understood that there are a variety of methods herein disclosed for determining the level of hybridization between two nucleic acid molecules. It is understood that these methods and conditions may provide different percentages of hybridization between two nucleic acid molecules, but unless otherwise indicated meeting the parameters of any of the methods would be sufficient. For example if 80% hybridization was required and as long as hybridization occurs within the required parameters in any one of these methods it is considered disclosed herein.

It is understood that those of skill in the art understand that if a composition or method meets any one of these criteria for determining hybridization either collectively or singly it is a composition or method that is disclosed herein.

### 6. Nucleic Acids

There are a variety of molecules disclosed herein that are nucleic acid based, including for example the nucleic acids that encode, for example, as well as various functional nucleic acids. The disclosed nucleic acids are made up of for example, nucleotides, nucleotide analogs, or nucleotide substitutes. Non-limiting examples of these and other molecules are discussed herein. It is understood that for example, when a vector is expressed in a cell, that the expressed mRNA will typically be made up of A, C, G, and U. Likewise, it is understood that if, for example, an antisense molecule is introduced into a cell or cell environment through for example exogenous delivery, it is advantagous that the antisense molecule be made up of nucleotide analogs that reduce the degradation of the antisense molecule in the cellular environment.

a) Nucleotides and Related Molecules

A nucleotide is a molecule that contains a base moiety, a sugar moiety and a phosphate moiety. Nucleotides can be linked together through their phosphate moieties and sugar moieties creating an internucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. A non-limiting example of a nucleotide would be 3′-AMP (3′-adenosine monophosphate) or 5′-GMP (5′-guanosine monophosphate).

A nucleotide analog is a nucleotide which contains some type of modification to either the base, sugar, or phosphate moieties. Modifications to nucleotides are well known in the art and would include for example, 5-methylcytosine (5-me-C), 5-hydroxymethyl cytosine, xanthine, hypoxanthine, and 2-aminoadenine as well as modifications at the sugar or phosphate moieties.

Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but which are linked together through a moiety other than a phosphate moiety. Nucleotide substitutes are able to conform to a double helix type structure when interacting with the appropriate target nucleic acid.

It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety. (Letsinger et al., Proc. Natl. Acad. Sci. USA, 1989,86, 6553-6556),

A Watson-Crick interaction is at least one interaction with the Watson-Crick face of a nucleotide, nucleotide analog, or nucleotide substitute. The Watson-Crick face of a nucleotide, nucleotide analog, or nucleotide substitute includes the C2, N1, and C6 positions of a purine based nucleotide, nucleotide analog, or nucleotide substitute and the C2, N3, C4 positions of a pyrimidine based nucleotide, nucleotide analog, or nucleotide substitute.

A Hoogsteen interaction is the interaction that takes place on the Hoogsteen face of a nucleotide or nucleotide analog, which is exposed in the major groove of duplex DNA. The Hoogsteen face includes the N7 position and reactive groups (NH2 or O) at the C6 position of purine nucleotides.

b) Sequences

There are a variety of sequences related to, for example, the E7 gene, the Akt gene, the p21^{Cip1} gene, and the Raf gene, and there encoded products, are set forth in SEQ ID Nos: 1-13. Also disclosed are fragments, functional fragments, and individual subsequences contained within these sequences. It is understood that there are a variety of allelic sequences as well as strain variants of the disclosed proteins and the nucleic acids that encode them. For example, oncogenic human papilloma virus (HPVs) contain a varity of strain variants sequences encoding for E7, but all of these proteins have a similar functional capability.

One particular sequence set forth in SEQ ID NO: 9, encoding E7 is used herein to exemplify the disclosed compositions and methods. It is understood that the description related to this sequence is applicable to any sequence related to SEQ ID NO:9 or the other disclosed sequences, unless specifically indicated otherwise. Those of skill in the art understand how to resolve sequence discrepancies and differences and to adjust the compositions and methods relating to a particular sequence to other related sequences (i.e. sequences of E7, Akt, p21^{Cip1}, or Raf). Primers and/or probes can be designed for any E7, Akt, p21^{Cip1}, or Raf sequence given the information disclosed herein and known in the art.

c) Primers and Probes

Disclosed are compositions including primers and probes, which are capable of interacting with the disclosed nucleic acids including those for p21^{Cip1}, Akt, E7, and Raf, as disclosed herein. In certain embodiments the primers are used to support DNA amplification reactions. Typically the primers will be capable of being extended in a sequence specific manner. Extension of a primer in a sequence specific manner includes any methods wherein the sequence and/or composition of the nucleic acid molecule to which the primer is hybridized or otherwise associated directs or influences the composition or sequence of the product produced by the extension of the primer. Extension of the primer in a sequence specific manner therefore includes, but is not limited to, PCR, DNA sequencing, DNA extension, DNA polymerization, RNA transcription, or reverse transcription. Techniques and conditions that amplify the primer in a sequence specific manner are preferred. In certain embodiments the primers are used for the DNA amplification reactions, such as PCR or direct sequencing. It is understood that in certain embodiments the primers can also be extended using non-enzymatic techniques, where for example, the nucleotides or oligonucleotides used to extend the primer are modified such that they will chemically react to extend the primer in a sequence specific manner. Typically the disclosed primers hybridize with the p21^{Cip1}, Akt, E7, and Raf genes, for example, or region of the p21^{Cip1}, Akt, E7, and Raf genes or they hybridize with the complement of the p21^{Cip1}, Akt, E7, and Raf genes, for example, or complement of a region of the p21^{Cip1}, Akt, E7, and Raf genes.

d) Functional Nucleic Acids

Functional nucleic acids are nucleic acid molecules that have a specific function, such as binding a target molecule or catalyzing a specific reaction. Functional nucleic acid molecules can be divided into the following categories, which are not meant to be limiting. For example, functional nucleic acids include antisense molecules, aptamers, ribozymes, triplex forming molecules, and external guide sequences. The functional nucleic acid molecules can act as affectors, inhibitors, modulators, and stimulators of a specific activity possessed by a target molecule, or the functional nucleic acid molecules can possess a de novo activity independent of any other molecules.

Functional nucleic acid molecules can interact with any macromolecule, such as DNA, RNA, polypeptides, or carbohydrate chains. Thus, functional nucleic acids can interact with the mRNA of, for example, E7 or Akt or the genomic DNA of E7 or Akt or they can interact with the E7 or Akt polypeptides. Often functional nucleic acids are designed to interact with other nucleic acids based on sequence homology between the target molecule and the functional nucleic acid molecule. In other situations, the specific recognition between the functional nucleic acid molecule and the target molecule is not based on sequence homology between the functional nucleic acid molecule and the target molecule, but rather is based on the formation of tertiary structure that allows specific recognition to take place.

Antisense molecules are designed to interact with a target nucleic acid molecule through either canonical or non-canonical base pairing. The interaction of the antisense molecule and the target molecule is designed to promote the destruction of the target molecule through, for example, RNAseH mediated RNA-DNA hybrid degradation. Alternatively the antisense molecule is designed to interrupt a processing function that normally would take place on the target molecule, such as transcription or replication. Antisense molecules can be designed based on the sequence of the target molecule. Numerous methods for optimization of antisense efficiency by finding the most accessible regions of the target molecule exist. Exemplary methods would be in vitro selection experiments and DNA modification studies using DMS and DEPC. It is preferred that antisense molecules bind the target molecule with a dissociation constant (k_{d})less than 10⁻⁶. It is more preferred that antisense molecules bind with a k_{d} less than 10⁻⁸. It is also more preferred that the antisense molecules bind the target moelcule with a k_{d} less than 10⁻¹⁰. It is also preferred that the antisense molecules bind the target molecule with a k_{d} less than 10⁻¹². A representative sample of methods and techniques which aid in the design and use of antisense molecules can be found in the following non-limiting list of U.S. Pat. Nos. 5,135,917, 5,294,533, 5,627,158, 5,641,754, 5,691,317, 5,780,607, 5,786,138, 5,849,903, 5,856,103, 5,919,772, 5,955,590, 5,990,088, 5,994,320, 5,998,602, 6,005,095, 6,007,995, 6,013,522, 6,017,898, 6,018,042, 6,025,198, 6,033,910, 6,040,296, 6,046,004, 6,046,319, and 6,057,437.

Aptamers are molecules that interact with a target molecule, preferably in a specific way. Typically aptamers are small nucleic acids ranging from 15-50 bases in length that fold into defined secondary and tertiary structures, such as stem-loops or G-quartets. Aptamers can bind small molecules, such as ATP (U.S. Pat. No. 5,631,146) and theophiline (U.S. Pat. No. 5,580,737), as well as large molecules, such as reverse transcriptase (U.S. Pat. No. 5,786,462) and thrombin (U.S. Pat. No. 5,543,293). Aptamers can bind very tightly with kds from the target molecule of less than 10⁻¹² M. It is preferred that the aptamers bind the target molecule with a k_{d} less than 10⁻⁶. It is more preferred that the aptamers bind the target molecule with a k_{d} less than 10⁻⁸. It is also more preferred that the aptamers bind the target molecule with a k_{d} less than 10⁻¹⁰. It is also preferred that the aptamers bind the target molecule with a k_{d} less than 10⁻¹². Aptamers can bind the target molecule with a very high degree of specificity. For example, aptamers have been isolated that have greater than a 10000 fold difference in binding affinities between the target molecule and another molecule that differ at only a single position on the molecule (U.S. Pat. No. 5,543,293). It is preferred that the aptamer have a k_{d} with the target molecule at least 10 fold lower than the k_{d} with a background binding molecule. It is more preferred that the aptamer have a k_{d} with the target molecule at least 100 fold lower than the k_{d} with a background binding molecule. It is more preferred that the aptamer have a k_{d} with the target molecule at least 1000 fold lower than the k_{d} with a background binding molecule. It is preferred that the aptamer have a k_{d} with the target molecule at least 10000 fold lower than the k_{d} with a background binding molecule. It is preferred when doing the comparison for a polypeptide for example, that the background molecule be a different polypeptide. For example, when determining the specificity of E7 or Akt aptamers, the background protein could be serum albumin. Representative examples of how to make and use aptamers to bind a variety of different target molecules can be found in the following non-limiting list of U.S. Pat. Nos. 5,476,766, 5,503,978, 5,631,146, 5,731,424, 5,780,228, 5,792,613, 5,795,721, 5,846,713, 5,858,660, 5,861,254, 5,864,026, 5,869,641, 5,958,691, 6,001,988, 6,011,020, 6,013,443, 6,020,130, 6,028,186, 6,030,776, and 6,051,698.

Ribozymes are nucleic acid molecules that are capable of catalyzing a chemical reaction, either intramolecularly or intermolecularly. Ribozymes are thus catalytic nucleic acid. It is preferred that the ribozymes catalyze intermolecular reactions. There are a number of different types of ribozymes that catalyze nuclease or nucleic acid polymerase type reactions which are based on ribozymes found in natural systems, such as hammerhead ribozymes, (for example, but not limited to the following U.S. Pat. Nos. 5,334,711, 5,436,330, 5,616,466, 5,633,133, 5,646,020, 5,652,094, 5,712,384, 5,770,715, 5,856,463, 5,861,288, 5,891,683, 5,891,684,5,985,621, 5,989,908, 5,998,193, 5,998,203, WO 9858058 by Ludwig and Sproat, WO 9858057 by Ludwig and Sproat, and WO 9718312 by Ludwig and Sproat) hairpin ribozymes (for example, but not limited to the following U.S. Pat. Nos. 5,631,115, 5,646,031, 5,683,902, 5,712,384, 5,856,188, 5,866,701, 5,869,339, and 6,022,962), and tetrahymena ribozymes (for example, but not limited to the following U.S. Pat. Nos. 5,595,873 and 5,652,107). There are also a number of ribozymes that are not found in natural systems, but which have been engineered to catalyze specific reactions de novo (for example, but not limited to the following U.S. Pat. Nos. 5,580,967, 5,688,670, 5,807,718, and 5,910,408). Preferred ribozymes cleave RNA or DNA substrates, and more preferably cleave RNA substrates. Ribozymes typically cleave nucleic acid substrates through recognition and binding of the target substrate with subsequent cleavage. This recognition is often based mostly on canonical or non-canonical base pair interactions. This property makes ribozymes particularly good candidates for target specific cleavage of nucleic acids because recognition of the target substrate is based on the target substrates sequence. Representative examples of how to make and use ribozymes to catalyze a variety of different reactions can be found in the following non-limiting list of U.S. Pat. Nos. 5,646,042,5,693,535, 5,731,295, 5,811,300, 5,837,855, 5,869,253, 5,877,021, 5,877,022, 5,972,699, 5,972,704, 5,989,906, and 6,017,756.

Triplex forming functional nucleic acid molecules are molecules that can interact with either double-stranded or single-stranded nucleic acid. When triplex molecules interact with a target region, a structure called a triplex is formed, in which there are three strands of DNA forming a complex dependant on both Watson-Crick and Hoogsteen base-pairing. Triplex molecules are preferred because they can bind target regions with high affinity and specificity. It is preferred that the triplex forming molecules bind the target molecule with a k_{d} less than 10⁻⁶. It is more preferred that the triplex forming molecules bind with a k_{d} less than 10⁻⁸. It is also more preferred that the triplex forming molecules bind the target moelcule with a k_{d} less than 10⁻¹⁰. It is also preferred that the triplex forming molecules bind the target molecule with a k_{d} less than 10⁻¹². Representative examples of how to make and use triplex forming molecules to bind a variety of different target molecules can be found in the following non-limiting list of U.S. Pat. Nos. 5,176,996, 5,645,985, 5,650,316,5,683,874, 5,693,773, 5,834,185, 5,869,246, 5,874,566, and 5,962,426.

External guide sequences (EGSs) are molecules that bind a target nucleic acid molecule forming a complex, and this complex is recognized by RNase P, which cleaves the target molecule. EGSs can be designed to specifically target a RNA molecule of choice. RNAse P aids in processing transfer RNA (tRNA) within a cell. Bacterial RNAse P can be recruited to cleave virtually any RNA sequence by using an EGS that causes the target RNA:EGS complex to mimic the natural tRNA substrate. (WO 92/03566 by Yale, and Forster and Altman, Science 238:407-409 (1990)).

Similarly, eukaryotic EGS/RNAse P-directed cleavage of RNA can be utilized to cleave desired targets within eukarotic cells. (Yuan et al., Proc. Natl. Acad. Sci. USA 89:8006-8010 (1992); WO 93/22434 by Yale; WO 95/24489 by Yale; Yuan and Altman, EMBO J 14:159-168 (1995), and Carrara et al., Proc. Natl. Acad. Sci. (USA) 92:2627-2631 (1995)). Representative examples of how to make and use EGS molecules to facilitate cleavage of a variety of different target molecules be found in the following non-limiting list of U.S. Pat. Nos. 5,168,053, 5,624,824, 5,683,873, 5,728,521, 5,869,248, and 5,877,162

### 7. Antibodies

a) Antibodies Generally

The term “antibodies” is used herein in a broad sense and includes both polyclonal and monoclonal antibodies. In addition to intact immunoglobulin molecules, also included in the term “antibodies” are fragments or polymers of those immunoglobulin molecules, and human or humanized versions of immunoglobulin molecules or fragments thereof, as long as they are chosen for their ability to interact with E7 or Akt such that the disclosed effect on p21^{Cip1}. The antibodies can be tested for their desired activity using the in vitro assays described herein, or by analogous methods, after which their in vivo therapeutic and/or prophylactic activities are tested according to known clinical testing methods. Also disclosed are functional equivalents of antibodies.

The term “monoclonal antibody” as used herein refers to an antibody obtained from a substantially homogeneous population of antibodies, i.e., the individual antibodies within the population are identical except for possible naturally occurring mutations that may be present in a small subset of the antibody molecules. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, as long as they exhibit the desired antagonistic activity (See, U.S. Pat. No. 4,816,567 and Morrison et al., Proc. Natl. Acad. Sci. USA, 81:6851-6855 (1984)).

The disclosed monoclonal antibodies can be made using any procedure which produces mono clonal antibodies. For example, monoclonal antibodies of the invention can be prepared using hybridoma methods, such as those described by Kohler and Milstein, Nature, 256:495 (1975). In a hybridoma method, a mouse or other appropriate host animal is typically immunized with an immunizing agent to elicit lymphocytes that produce or are capable of producing antibodies that will specifically bind to the immunizing agent.

The monoclonal antibodies may also be made by recombinant DNA methods, such as those described in U.S. Pat. No. 4,816,567 (Cabilly et al.). DNA encoding the disclosed monoclonal antibodies can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques, e.g., as described in U.S. Pat. No. 5,804,440 to Burton et al. and U.S. Pat. No. 6,096,441 to Barbas et al.

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348 published Dec. 22, 1994 and U.S. Pat. No. 4,342,566. Papain digestion of antibodies typically produces two identical antigen binding fragments, called Fab fragments, each with a single antigen binding site, and a residual Fc fragment. Pepsin treatment yields a fragment that has two antigen combining sites and is still capable of cross-linking antigen.

The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, M. J. Curr. Opin. Biotechnol. 3:348-354, 1992).

As used herein, the term “antibody” or “antibodies” can also refer to a human antibody and/or a humanized antibody. Many non-human antibodies (e.g., those derived from mice, rats, or rabbits) are naturally antigenic in humans, and thus can give rise to undesirable immune responses when administered to humans. Therefore, the use of human or humanized antibodies in the methods of the invention serves to lessen the chance that an antibody administered to a human will evoke an undesirable immune response.

b) Human Antibodies

The human antibodies of the invention can be prepared using any technique. Examples of techniques for human monoclonal antibody production include those described by Cole et al. (Monoclonal Antibodies and Cancer Therapy, Alan R. Liss, p. 77, 1985) and by Boemer et al. (J. Immunol., 147(1):86-95, 1991). Human antibodies of the invention (and fragments thereof) can also be produced using phage display libraries (Hoogenboom et al., J. Mol. BioL, 227:381, 1991; Marks et al., J. Mol. Biol., 222:581, 1991).

The human antibodies of the invention can also be obtained from transgenic animals. For example, transgenic, mutant mice that are capable of producing a full repertoire of human antibodies, in response to immunization, have been described (see, e.g., Jakobovits et al., Proc. Natl. Acad. Sci. USA, 90:2551-255 (1993); Jakobovits et al., Nature, 362:255-258 (1993); Bruggermann et al., Year in Immunol., 7:33 (1993)). Specifically, the homozygous deletion of the antibody heavy chain joining region (J(H)) gene in these chimeric and germ-line mutant mice results in complete inhibition of endogenous antibody production, and the successful transfer of the human germ-line antibody gene array into such germ-line mutant mice results in the production of human antibodies upon antigen challenge.

c) Humanized Antibodies

Antibody humanization techniques generally involve the use of recombinant DNA technology to manipulate the DNA sequence encoding one or more polypeptide chains of an antibody molecule. Accordingly, a humanized form of a non-human antibody (or a fragment thereof) is a chimeric antibody or antibody chain (or a fragment thereof, such as an Fv, Fab, Fab′, or other antigen-binding portion of an antibody) which contains a portion of an antigen binding site from a non-human (donor) antibody integrated into the framework of a human (recipient) antibody.

To generate a humanized antibody, residues from one or more complementarity determining regions (CDRs) of a recipient (human) antibody molecule are replaced by residues from one or more CDRs of a donor (non-human) antibody molecule that is known to have desired antigen binding characteristics (e.g., a certain level of specificity and affinity for the target antigen). In some instances, Fv framework (FR) residues of the human antibody are replaced by corresponding non-human residues. Humanized antibodies may also contain residues which are found neither in the recipient antibody nor in the imported CDR or framework sequences. Generally, a humanized antibody has one or more amino acid residues introduced into it from a source which is non-human. In practice, humanized antibodies are typically human antibodies in which some CDR residues and possibly some FR residues are substituted by residues from analogous sites in rodent antibodies. Humanized antibodies generally contain at least a portion of an antibody constant region (Fc), typically that of a human antibody (Jones et al., Nature, 321:522-525 (1986), Reichmann et al., Nature, 332:323-327 (1988), and Presta, Curr. Opin. Struct. Biol., 2:593-596 (1992)).

Methods for humanizing non-human antibodies are well known in the art. For example, humanized antibodies can be generated according to the methods of Winter and co-workers (Jones et al., Nature, 321:522-525 (1986), Riechmann et al., Nature, 332:323-327 (1988), Verhoeyen et al., Science, 239:1534-1536 (1988)), by substituting rodent CDRs or CDR sequences for the corresponding sequences of a human antibody. Methods that can be used to produce humanized antibodies are also described in U.S. Pat. No. 4,816,567 (Cabilly et al.), U.S. Pat. No. 5,565,332 (Hoogenboom et al.), U.S. Pat. No. 5,721,367 (Kay et al.), U.S. Pat. No. 5,837,243 (Deo et al.), U.S. Pat. No. 5,939,598 (Kucherlapati et al.), U.S. Pat. No. 6,130,364 (Jakobovits et al.), and U.S. Pat. No. 6,180,377 (Morgan et al.).

d) Administration of Antibodies

Administration of the antibodies can be done as disclosed herein. Nucleic acid approaches for antibody delivery also exist. The broadly neutralizing anti E7 or Akt antibodies and antibody fragment, or other antibodies antibodies and antibody fragments disclosed herein, can also be administered to patients or subjects as a nucleic acid preparation (e.g., DNA or RNA) that encodes the antibody or antibody fragment, such that the patient's or subject's own cells take up the nucleic acid and produce and secrete the encoded antibody or antibody fragment. The delivery of the nucleic acid can be by any means, as disclosed herein, for example.

### 8. Peptides

a) Protein Variants

As discussed herein there are numerous variants of the E7 protein and Akt protein or the p21^{Cip1} and Raf proteins, for example, that are known and herein contemplated. In addition, to the known functional E7, Akt, p21^{Cip1}, and Raf strain variants, for example, there are derivatives of the E7, Akt, p21^{Cip1}, and Raf proteins, for example, which also function in the disclosed methods and compositions. Protein variants and derivatives are well understood to those of skill in the art and in can involve amino acid sequence modifications. For example, amino acid sequence modifications typically fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Immunogenic fusion protein derivatives, such as those described in the examples, are made by fusing a polypeptide sufficiently large to confer immunogenicity to the target sequence by cross-linking in vitro or by recombinant cell culture transformed with DNA encoding the fusion. Deletions are characterized by the removal of one or more amino acid residues from the protein sequence. Typically, no more than about from 2 to 6 residues are deleted at any one site within the protein molecule. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known, for example M13 primer mutagenesis and PCR mutagenesis. Amino acid substitutions are typically of single residues, but can occur at a number of different locations at once; insertions usually will be on the order of about from 1 to 10 amino acid residues; and deletions will range about from 1 to 30 residues. Deletions or insertions preferably are made in adjacent pairs, i.e. a deletion of 2 residues or insertion of 2 residues. Substitutions, deletions, insertions or any combination thereof may be combined to arrive at a final construct. The mutations must not place the sequence out of reading frame and preferably will not create complementary regions that could produce secondary mRNA structure. Substitutional variants are those in which at least one residue has been removed and a different residue inserted in its place. Such substitutions generally are made in accordance with the following Tables 1 and 2 and are referred to as conservative substitutions.

TABLE 1
Amino Acid Abbreviations

| Amino Acid | Abbreviations |
|---|---|
| Alanine | AlaA |
| Allosoleucine | AIle |
| Arginine | ArgR |
| Asparagines | AsnN |
| aspartic acid | AspD |
| Cysteine | CysC |
| glutamic acid | GluE |
| Glutamine | GlnK |
| Glycine | GlyG |
| Histidine | HisH |
| Isolelucine | IleI |
| Leucine | LeuL |
| Lysine | LysK |
| Phenylalanine | PheF |
| Proline | ProP |
| pyroglutamic acidp | Glu |
| Serine | SerS |
| Threonine | ThrT |
| Tyrosine | TyrY |
| Tryptophan | TrpW |
| Valine | ValV |

TABLE 2

Amino Acid Substitutions Exemplary Conservative
Substitutions, others are known in the art. Either
can be substituted for the other

| | |
|---|---|
| Ala | ser |
| Ar | glys, gln |
| Asn | gln; his |
| Asp | glu |
| Cys | ser |
| Gln | asn, lys |
| Glu | asp |
| Gly | ala |
| His | asn; gln |
| Ile | leu; val |
| Leu | ile; val |
| Lys | arg; gln; |
| Met | Leu; ile |
| Phe | met; leu; tyr |
| Ser | thr |
| Thr | ser |
| Trp | tyr |
| Tyr | trp; phe |
| Val | ile; leu |

Substantial changes in function or immunological identity are made by selecting substitutions that are less conservative than those in Table 2, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, lle, Leu; Asp, Glu; Asn, Gln; Ser, Thr; Lys, Arg; and Phe, Tyr. Such conservatively substituted variations of each explicitly disclosed sequence are included within the mosaic polypeptides provided herein.

Substitutional or deletional mutagenesis can be employed to insert sites for N-glycosylation (Asn-X-Thr/Ser) or O-glycosylation (Ser or Thr). Deletions of cysteine or other labile residues also may be desirable. Deletions or substitutions of potential proteolysis sites, e.g. Arg, is accomplished for example by deleting one of the basic residues or substituting one by glutaminyl or histidyl residues.

Certain post-translational derivatizations are the result of the action of recombinant host cells on the expressed polypeptide. Glutaminyl and asparaginyl residues are frequently post-translationally deamidated to the corresponding glutamyl and asparyl residues. Alternatively, these residues are deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains (T. E. Creighton, Proteins: Structure and Molecular Properties, W. H. Freeman & Co., San Francisco pp 79-86 [1983]), acetylation of the N-terminal amine and, in some instances, amidation of the C-terminal carboxyl.

It is understood that one way to define the variants and derivatives of the disclosed proteins herein is through defining the variants and derivatives in terms of homology/identity to specific known sequences. For example, SEQ ID NOs:1-4, 6, 8, and 11 set forth particular sequences of disclosed proteins. Specifically disclosed are variants of these and other proteins herein disclosed which have at least, 70% or 75% or 80% or 85% or 90% or 95% homology to the stated sequence. Those of skill in the art readily understand how to determine the homology of two proteins. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment.

It is understood that the description of conservative mutations and homology can be combined together in any combination, such as embodiments that have at least 70% homology to a particular sequence wherein the variants are conservative mutations.

As this specification discusses various proteins and protein sequences it is understood that the nucleic acids that can encode those protein sequences are also disclosed. This would include all degenerate sequences related to a specific protein sequence, i.e. all nucleic acids having a sequence that encodes one particular protein sequence as well as all nucleic acids, including degenerate nucleic acids, encoding the disclosed variants and derivatives of the protein sequences. Thus, while each particular nucleic acid sequence may not be written out herein, it is understood that each and every sequence is in fact disclosed and described herein through the disclosed protein sequence. For example, one of the many nucleic acid sequences that can encode the protein sequence set foth in SEQ ID NO:8 is set forth in SEQ ID NO:9. Another nucleic acid sequence that encodes the same protein sequence set forth in SEQ ID NO:8 is set forth in SEQ ID NO:10. In addition, for example, a disclosed conservative derivative of SEQ ID NO:8 is shown in SEQ ID NO: 11, where the isoleucine (1) at position 38 is changed to a valine (V). It is understood that for this mutation all of the nucleic acid sequences that encode this particular derivative of the E7 protein are also disclosed including for example SEQ ID NO:12 and SEQ ID NO:13 which set forth two of the nucleic acid sequences that encode the particular polypeptide set forth in SEQ ID NO: I1. It is also understood that while no amino acid sequence indicates what particular DNA sequence encodes that protein within an organism, where particular variants of a disclosed protein are disclosed herein, the known nucleic acid sequence that encodes that protein in the particular strain or species from which that protein arises is also known and herein disclosed and described.

It is understood that there are numerous amino acid and peptide analogs which can be incorporated into the disclosed compositions. For example, there are numerous D amino acids or amino acids which have a different functional substituent then the amino acids shown in Table 1 and Table 2. The opposite stereo isomers of naturally occurring peptides are disclosed, as well as the stereo isomers of peptide analogs. These amino acids can readily be incorporated into polypeptide chains by charging tRNA molecules with the amino acid of choice and engineering genetic constructs that utilize, for example, amber codons, to insert the analog amino acid into a peptide chain in a site specific way (Thorson et al., Methods in Molec. Biol. 77:43-73 (1991), Zoller, Current Opinion in Biotechnology, 3:348-354 (1992); Ibba, Biotechnology & Genetic Enginerring Reviews 13:197-216 (1995), Cahill et al., TIBS, 14(10):400403 (1989); Benner, TIB Tech, 12:158-163 (1994); Ibba and Hennecke, Bio/technology, 12:678-682 (1994) all of which are herein incorporated by reference at least for material related to amino acid analogs).

Molecules can be produced that resemble peptides, but which are not connected via a natural peptide linkage. For example, linkages for amino acids or amino acid analogs can include CH₂NH—, —CH₂S—, —CH₂—CH₂—, —CH═CH— (cis and trans), —COCH₂—, —CH(OH)CH₂—, and —CHH₂SO—(These and others can be found in Spatola, A. F. in Chemistry and Biochemistry of Amino Acids, Peptides, and Proteins, B. Weinstein, eds., Marcel Dekker, New York, p. 267 (1983); Spatola, A. F., Vega Data (March 1983), Vol. 1, Issue 3, Peptide Backbone Modifications (general review); Morley, Trends Pharm Sci (1980) pp. 463-468; Hudson, D. et al., Int J Pept Prot Res 14:177-185 (1979) (—CH₂NR—, CH₂CH₂—); Spatola et al. Life Sci 38:1243-1249 (1986) (—CH H₂—S); Hann J. Chem. Soc Perkin Trans. [307-314 (1982) (—CH—CH—, cis and trans); Almquist et al. J. Med. Chem. 23:1392-1398 (1980) (—COCH₂—); Jennings-White et al. Tetrahedron Lett 23:2533 (1982) (—COCH₂—); Szelke et al. European Appln, EP 45665 CA (1982): 97:39405 (1982) (—CH(OH)CH₂—); Holladay et al. Tetrahedron. Lett 24:4401-4404 (1983) (—C(OH)CH₂—); and Hruby Life Sci 31:189-199 (1982) (—CH₂—S—); each of which is incorporated herein by reference. A particularly preferred non-peptide linkage is —CH₂NH—. It is understood that peptide analogs can have more than one atom between the bond atoms, such as b-alanine, g-aminobutyric acid, and the like.

Amino acid analogs and analogs and peptide analogs often have enhanced or desirable properties, such as, more economical production, greater chemical stability, enhanced pharmacological properties (half-life, absorption, potency, efficacy, etc.), altered specificity (e.g., a broad-spectrum of biological activities), reduced antigenicity, and others.

D-amino acids can be used to generate more stable peptides, because D amino acids are not recognized by peptidases and such. Systematic substitution of one or more amino acids of a consensus sequence with a D-amino acid of the same type (e.g., D-lysine in place of L-lysine) can be used to generate more stable peptides. Cysteine residues can be used to cyclize or attach two or more peptides together. This can be beneficial to constrain peptides into particular conformations. (Rizo and Gierasch Ann. Rev. Biochem. 61:387 (1992), incorporated herein by reference).

### 9. Delivery of the Compositions to Cells

a) Nucleic Acid Delivery

There are a number of compositions and methods which can be used to deliver nucleic acids to cells, either in vitro or in vivo. These methods and compositions can largely be broken down into two classes: viral based delivery systems and non-viral based delivery systems. For example, the nucleic acids can be delivered through a number of direct delivery systems such as, electroporation, lipofection, calcium phosphate precipitation, plasmids, viral vectors, viral nucleic acids, phage nucleic acids, phages, cosmids, or via transfer of genetic material in cells or carriers such as cationic liposomes. Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991) Such methods are well known in the art and readily adaptable for use with the compositions and methods described herein. In certain cases, the methods will be modifed to specifically function with large DNA molecules. Further, these methods can be used to target certain diseases and cell populations by using the targeting characteristics of the carrier.

In the methods described herein, which include the administration and uptake of exogenous DNA into the cells of a subject (i.e., gene transduction or transfection), the nucleic acids of the present invention can be in the form of naked DNA or RNA, or the nucleic acids can be in a vector for delivering the nucleic acids to the cells, whereby the encoding DNA or DNA or fragment is under the transcriptional regulation of a promoter, as would be well understood by one of ordinary skill in the art as well as enhancers. The vector can be a commercially available preparation, such as an adenovirus vector (Quantum Biotechnologies, Inc. (Laval, Quebec, Canada).

As one example, vector delivery can be via a viral system, such as a retroviral vector system which can package a recombinant retroviral genome (see e.g., Pastan et al., Proc. Natl. Acad. Sci. U.S.A. 85:4486, 1988; Miller et al., Mol. Cell. Biol. 6:2895, 1986). The recombinant retrovirus can then be used to infect and thereby deliver to the infected cells nucleic acid encoding a broadly neutralizing antibody (or active fragment thereof) of the invention. The exact method of introducing the altered nucleic acid into mammalian cells is, of course, not limited to the use of retroviral vectors. Other techniques are widely available for this procedure including the use of adenoviral vectors (Mitani et al., Hum. Gene Ther. 5:941-948, 1994), adeno-associated viral (AAV) vectors (Goodman et al., Blood 84:1492-1500, 1994), lentiviral vectors (Naidini et al., Science 272:263-267, 1996), pseudotyped retroviral vectors (Agrawal et al., Exper. Hematol. 24:738-747, 1996). Physical transduction techniques can also be used, such as liposome delivery and receptor-mediated and other endocytosis mechanisms (see, for example, Schwartzenberger et al., Blood 87:472-478, 1996). This invention can be used in conjunction with any of these or other commonly used gene transfer methods.

As one example, if the antibody-encoding nucleic acid or some other other nucleic acid encoding an inhibitor of the E7 or Akt proteins or encoding a particular variant of the E7 or Akt genes to be used in the disclosed methods, is delivered to the cells of a subject in an adenovirus vector, the dosage for administration of adenovirus to humans can range from about 10⁷ to 10⁹ plaque forming units (pfu) per injection but can be as high as 10¹² pfu per injection (Crystal, Hum. Gene Ther. 8:985-1001, 1997; Alvarez and Curiel, Hum. Gene Ther. 8:597-613, 1997). A subject can receive a single injection, or, if additional injections are necessary, they can be repeated at six month intervals (or other appropriate time intervals, as determined by the skilled practitioner) for an indefinite period and/or until the efficacy of the treatment has been established.

Parenteral administration of the nucleic acid or vector of the present invention, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein. For additional discussion of suitable formulations and various routes of administration of therapeutic compounds, see, e.g., Remington: The Science and Practice of Pharmacy (19th ed.) ed. A. R. Gennaro, Mack Publishing Company, Easton, Pa. 1995.

Nucleic acids that are delivered to cells which are to be integrated into the host cell genome, typically contain integration sequences. These sequences are often viral related sequences, particularly when viral based systems are used. These viral intergration systems can also be incorporated into nucleic acids which are to be delivered using a non-nucleic acid based system of deliver, such as a liposome, so that the nucleic acid contained in the delivery system can become integrated into the host genome.

Other general techniques for integration into the host genome include, for example, systems designed to promote homologous recombination with the host genome. These systems typically rely on sequence flanking the nucleic acid to be expressed that has enough homology with a target sequence within the host cell genome that recombination between the vector nucleic acid and the target nucleic acid takes place, causing the delivered nucleic acid to be integrated into the host genome. These systems and the methods necessary to promote homologous recombination are known to those of skill in the art.

b) Non-Nucleic Acid Based Systems

The disclosed compositions can be delivered to the target cells in a variety of ways. For example, the compositions can be delivered through electroporation, or through lipofection, or through calcium phosphate precipitation. The delivery mechanism chosen will depend in part on the type of cell targeted and whether the delivery is occurring for example in vivo or in vitro.

Thus, the compositions can comprise, in addition to the disclosed compositions or vectors for example, lipids such as liposomes, such as cationic liposomes (e.g., DOTMA, DOPE, DC-cholesterol) or anionic liposomes. Liposomes can further comprise proteins to facilitate targeting a particular cell, if desired. Administration of a composition comprising a compound and a cationic liposome can be administered to the blood afferent to a target organ or inhaled into the respiratory tract to target cells of the respiratory tract. Regarding liposomes, see, e.g., Brigham et al. Am. J. Resp. Cell. Mol. Biol. 1:95-100 (1989); FeIgner et al. Proc. Natl. Acad. Sci USA 84:7413-7417 (1987); U.S. Pat. No. 4,897,355. Furthermore, the compound can be administered as a component of a microcapsule that can be targeted to specific cell types, such as macrophages, or where the diffusion of the compound or delivery of the compound from the microcapsule is designed for a specific rate or dosage.

In the methods described above which include the administration and uptake of exogenous DNA into the cells of a subject (i.e., gene transduction or transfection), delivery of the compositions to cells can be via a variety of mechanisms. As one example, delivery can be via a liposome, using commercially available liposome preparations such as LIPOFECTIN, LIPOFECTAMINE (GIBCO-BRL, Inc., Gaithersburg, Md.), SUPERFECT (Qiagen, Inc. Hilden, Germany) and TRANSFECTAM (Promega Biotec, Inc., Madison, Wis.), as well as other liposomes developed according to procedures standard in the art. In addition, the nucleic acid or vector of this invention can be delivered in vivo by electroporation, the technology for which is available from Genetronics, Inc. (San Diego, Calif.) as well as by means of a SONOPORATION machine (ImaRx Pharmaceutical Corp., Tucson, Ariz.).

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). These techniques can be used for a variety of other specific cell types. Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1 992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

c) In vivo/ex vivo

As described above, the compositions can be administered in a pharnmaceutically acceptable carrier and can be delivered to the subject=s cells in vivo and/or ex vivo by a variety of mechanisms well known in the art (e.g., uptake of naked DNA, liposome fusion, intramuscular injection of DNA via a gene gun, endocytosis and the like).

If ex vivo methods are employed, cells or tissues can be removed and maintained outside the body according to standard protocols well known in the art. The compositions can be introduced into the cells via any gene transfer mechanism, such as, for example, calcium phosphate mediated gene delivery, electroporation, microinjection or proteoliposomes. The transduced cells can then be infused (e.g., in a pharmaceutically acceptable carrier) or homotopically transplanted back into the subject per standard methods for the cell or tissue type. Standard methods are known for transplantation or infusion of various cells into a subject.

### 10. Expression Systems

The nucleic acids that are delivered to cells typically contain expression controlling systems. For example, the inserted genes in viral and retroviral systems usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

a) Viral Promoters and Enhancers

Promoters controlling transcription from vectors in mammalian host cells may be obtained from various sources, for example, the genomes of viruses such as: polyoma, Simian Virus 40 (SV40), adenovirus, retroviruses, hepatitis-B virus and most preferably cytomegalovirus, or from heterologous mammalian promoters, e.g. beta actin promoter. The early and late promoters of the SV40 virus are conveniently obtained as an SV40 restriction fragment which also contains the SV40 viral origin of replication (Fiers et al., Nature, 273: 113 (1978)). The immediate early promoter of the human cytomegalovirus is conveniently obtained as a HindIII E restriction fragment (Greenway, P. J. et al., Gene 18: 355-360 (1982)). Of course, promoters from the host cell or related species also are useful herein.

Enhancer generally refers to a sequence of DNA that functions at no fixed distance from the transcription start site and can be either 5′ (Laimins, L. et al., Proc. Natl. Acad. Sci. 78: 993 (1981)) or 3′ (Lusky, M. L., et al., Mol. Cell Bio. 3: 1108 (1983)) to the transcription unit. Furthermore, enhancers can be within an intron (Banerdi, J. L. et al., Cell 33: 729 (1983)) as well as within the coding sequence itself (Osborne, T. F., et al., Mol. Cell Bio. 4: 1293 (1984)). They are usually between 10 and 300 bp in length, and they function in cis. Enhancers f unction to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, -fetoprotein and insulin), typically one will use an enhancer from a eukaryotic cell virus for general expression. Preferred examples are the SV40 enhancer on the late side of the replication origin (bp 100-270), the cytomegalovirus early promoter enhancer, the polyoma enhancer on the late side of the replication origin, and adenovirus enhancers.

The promotor and/or enhancer may be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

In certain embodiments the promoter and/or enhancer region can act as a constitutive promoter and/or enhancer to maximize expression of the region of the transcription unit to be transcribed. In certain constructs the promoter and/or enhancer region be active in all eukaryotic cell types, even if it is only expressed in a particular type of cell at a particular time. A preferred promoter of this type is the CMV promoter (650 bases). Other preferred promoters are SV40 promoters, cytomegalovirus (full length promoter), and retroviral vector LTF.

It has been shown that all specific regulatory elements can be cloned and used to construct expression vectors that are selectively expressed in specific cell types such as melanoma cells. The glial fibrillary acetic protein (GFAP) promoter has been used to selectively express genes in cells of glial origin.

Expression vectors used in eukaryotic host cells (yeast, fungi, insect, plant, animal, human or nucleated cells) may also contain sequences necessary for the termination of transcription which may affect mRNA expression. These regions are transcribed as polyadenylated segments in the untranslated portion of the mRNA encoding tissue factor protein. The 3′ untranslated regions also include transcription termination sites. It is preferred that the transcription unit also contain a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs. In certain transcription units, the polyadenylation region is derived from the SV40 early polyadenylation signal and consists of about 400 bases. It is also preferred that the transcribed units contain other standard sequences alone or in combination with the above sequences improve expression from, or stability of, the construct.

b) Markers

The viral vectors can include nucleic acid sequence encoding a marker product. This marker product is used to determine if the gene has been delivered to the cell and once delivered is being expressed. Preferred marker genes are the E. Coli lacZ gene, which encodes β-galactosidase, and green fluorescent protein.

In some embodiments the marker may be a selectable marker. Examples of suitable selectable markers for mammalian cells are dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two examples are: CHO DHFR- cells and mouse LTK-cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non-supplemented media.

The second category is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin, (Southern P. and Berg, P., J. Molec. Appl. Genet. 1: 327 (1982)), mycophenolic acid, (Mulligan, R. C. and Berg, P. Science 209: 1422 (1980)) or hygromycin, (Sugden, B. et al., Mol. Cell. Biol. 5: 410-413 (1985)). The three examples employ bacterial genes under eukaryotic control to convey resistance to the appropriate drug G418 or neomycin (geneticin), xgpt (mycophenolic acid) or hygromycin, respectively. Others include the neomycin analog G418 and puramycin.

### 11. Pharmaceutical Carriers/Delivery of Pharmaceutical Products

As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like, although topical intranasal administration or administration by inhalant is typically preferred. As used herein, “topical intranasal administration” means delivery of the compositions into the nose and nasal passages through one or both of the nares and can comprise delivery by a spraying mechanism or droplet mechanism, or through aerosolization of the nucleic acid or vector. The latter may be effective when a large number of animals is to be treated simultaneously. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research,49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

a) Pharmaceutically Acceptable Carriers

The compositions, including antibodies, can be used therapeutically in combination with a pharmaceutically acceptable carrier.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceu Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

b) Therapeutic Uses

The dosage ranges for the administration of the compositions are those large enough to produce the desired effect in which the symptoms disorder are effected. The dosage should not be so large as to cause adverse side effects, such as unwanted cross-reactions, anaphylactic reactions, and the like. Generally, the dosage will vary with the age, condition, sex and extent of the disease in the patient and can be determined by one of skill in the art. The dosage can be adjusted by the individual physician in the event of any counterindications. Dosage can vary, and can be administered in one or more dose administrations daily, for one or several days.

### 12. Chips and Micro Arrays

Disclosed are chips where at least one address is the sequences or part of the sequences set forth in any of the nucleic acid sequences disclosed herein. Also disclosed are chips where at least one address is the sequences or portion of sequences set forth in any of the peptide sequences disclosed herein.

Also disclosed are chips where at least one address is a variant of the sequences or part of the sequences set forth in any of the nucleic acid sequences disclosed herein. Also disclosed are chips where at least one address is a variant of the sequences or portion of sequences set forth in any of the peptide sequences disclosed herein.

Disclosed are chips comprising E7 protein and Akt protein or the p21^{Cip1} and Raf protein (or nucleic acid related to the same) which are designed such that the chips can be used to identify molecules having the properties disclosed herein. Also disclosed are chips where the chips comprise molecules that interact with either E7 protein and Akt protein or the p21^{Cip1} and Raf protein.

### 13. Computer Readable Mediums

It is understood that the disclosed nucleic acids and proteins can be represented as a sequence consisting of the nucleotides of amino acids, and that the relationships between protein molecules can be stored in a computer readable medium. There are a variety of ways to display these sequences or molecule relationships, such as the relationship between E7, Akt, Raf, and p21^{Cip1}, for example the nucleotide guanosine can be represented by G or g and the amino acid valine can be represented by Val or V, and the proteins themselves could be stored as, for example, their sequence or by representations of the protein, such as a defining word or symbol. Those of skill in the art understand how to display and express any nucleic acid or protein sequence or protein relationship in any of the variety of ways that exist, each of which is considered herein disclosed. Specifically contemplated herein is the display or storage of these sequences or molecule relationships on computer readable mediums, such as, commercially available floppy disks, tapes, chips, hard drives, compact disks, and video disks, or other computer readable mediums. Also disclosed are the binary code representations of the disclosed sequences and the disclosed molecule relationships. Those of skill in the art understand what computer readable mediums. Thus, computer readable mediums on which the nucleic acids or protein sequences are recorded, stored, or saved.

Disclosed are computer readable mediums comprising the sequences and/or molecule relationships and information regarding the sequences and/or molecule relationships set forth herein, as well as the relationships between the proteins disclosed in the mechanisms disclosed herein.

### 14. Kits

Disclosed herein are kits that are drawn to reagents that can be used in practicing the methods disclosed herein. The kits can include any reagent or combination of reagent discussed herein or that would be understood to be required or beneficial in the practice of the disclosed methods. For example, the kits could include primers to perform the amplification reactions discussed in certain embodiments of the methods, as well as the buffers and enzymes required to use the primers as intended. The kits could also include, for example, cells that are deisigned as disclosed herein, for use in screening or testing the activity of compounds that modulate the effect of E7 on a cell.

### C. METHODS OF MAKING THE COMPOSITIONS

The compositions disclosed herein and the compositions necessary to perform the disclosed methods can be made using any method known to those of skill in the art for that particular reagent or compound unless otherwise specifically noted.

### 1. Nucleic Acid Synthesis

For example, the nucleic acids, such as, the oligonucleotides to be used as primers can be made using standard chemical synthesis methods or can be produced using enzymatic methods or any other known method. Such methods can range from standard enzymatic digestion followed by nucleotide fragment isolation (see for example, Sambrook et al., Molecular Cloning: A Laboratory Manual, 2nd Edition (Cold Spring Harbor Laboratory Press, Cold Spring Harbor, N.Y., 1989) Chapters 5, 6) to purely synthetic methods, for example, by the cyanoethyl phosphoramidite method using a Milligen or Beckman System 1Plus DNA synthesizer (for example, Model 8700 automated synthesizer of Milligen-Biosearch, Burlington, Mass. or ABI Model 380B). Synthetic methods useful for making oligonucleotides are also described by Ikuta et al., Ann. Rev. Biochem. 53:323-356 (1984), (phosphotriester and phosphite-triester methods), and Narang et al., Methods Enzymol., 65:610-620 (1980), (phosphotriester method). Protein nucleic acid molecules can be made using known methods such as those described by Nielsen et al., Bioconjug. Chem. 5:3-7 (1994).

### 2. Peptide Synthesis

One method of producing the disclosed proteins, such as SEQ, is to link two or more peptides or polypeptides together by protein chemistry techniques. For example, peptides or polypeptides can be chemically synthesized using currently available laboratory equipment using either Fmoc (9-fluorenylmethyloxycarbonyl) or Boc (tert-butyloxycarbonoyl) chemistry. (Applied Biosystems, Inc., Foster City, Calif.). One skilled in the art can readily appreciate that a peptide or polypeptide corresponding to the disclosed proteins, for example, can be synthesized by standard chemical reactions. For example, a peptide or polypeptide can be synthesized and not cleaved from its synthesis resin whereas the other fragment of a peptide or protein can be synthesized and subsequently cleaved from the resin, thereby exposing a terminal group which is functionally blocked on the other fragment. By peptide condensation reactions, these two fragments can be covalently joined via a peptide bond at their carboxyl and amino termini, respectively, to form an antibody, or fragment thereof. (Grant G A (1992) Synthetic Peptides: A User Guide. W.H. Freeman and Co., N.Y. (1992); Bodansky M and Trost B., Ed. (1 993) Principles of Peptide Synthesis. Springer-Verlag Inc., NY (which is herein incorporated by reference at least for material related to peptide synthesis). Alternatively, the peptide or polypeptide is independently synthesized in vivo as described herein. Once isolated, these independent peptides or polypeptides may be linked to form a peptide or fragment thereof via similar peptide condensation reactions.

For example, enzymatic ligation of cloned or synthetic peptide segments allow relatively short peptide fragments to be joined to produce larger peptide fragments, polypeptides or whole protein domains (Abrahmsen L et al., Biochemistry, 30:4151 (1991)). Alternatively, native chemical ligation of synthetic peptides can be utilized to synthetically construct large peptides or polypeptides from shorter peptide fragments. This method consists of a two step chemical reaction (Dawson et al. Synthesis of Proteins by Native Chemical Ligation. Science, 266:776-779 (1994)). The first step is the chemoselective reaction of an unprotected synthetic peptide—thioester with another unprotected peptide segment containing an amino-terminal Cys residue to give a thioester-linked intermediate as the initial covalent product. Without a change in the reaction conditions, this intermediate undergoes spontaneous, rapid intramolecular reaction to form a native peptide bond at the ligation site (Baggiolini M et al. (1992) FEBS Lett. 307:97-101; Clark-Lewis I et al., J.Biol.Chem., 269:16075 (1994); Clark-Lewis I et al., Biochemistry, 30:3128 (1991); Rajarathnam K et al., Biochemistry 33:6623-30 (1994)).

Alternatively, unprotected peptide segments are chemically linked where the bond formed between the peptide segments as a result of the chemical ligation is an unnatural (non-peptide) bond (Schnolzer, M et al. Science, 256:221 (1992)). This technique has been used to synthesize analogs of protein domains as well as large amounts of relatively pure proteins with full biological activity (deLisle Milton R C et al., Techniques in Protein Chemistry IV. Academic Press, New York, pp. 257-267 (1992)).

### 3. Process for Making the Compositions

Disclosed are processes for making the compositions as well as making the intermediates leading to the compositions. There are a variety of methods that can be used for making these compositions, such as synthetic chemical methods and standard molecular biology methods. It is understood that the methods of making these and the other disclosed compositions are specifically disclosed.

Disclosed are cells produced by the process of transforming the cell with any of the disclosed nucleic acids. Disclosed are cells produced by the process of transforming the cell with any of the non-naturally occurring disclosed nucleic acids.

Disclosed are any of the disclosed peptides produced by the process of expressing any of the disclosed nucleic acids. Disclosed are any of the non-naturally occurring disclosed peptides produced by the process of expressing any of the disclosed nucleic acids. Disclosed are any of the disclosed peptides produced by the process of expressing any of the non-naturally disclosed nucleic acids.

Disclosed are animals produced by the process of transfecting a cell within the animal with any of the nucleic acid molecules disclosed herein. Disclosed are animals produced by the process of transfecting a cell within the animal any of the nucleic acid molecules disclosed herein, wherein the animal is a mammal. Also disclosed are animals produced by the process of transfecting a cell within the animal any of the nucleic acid molecules disclosed herein, wherein the mammal is mouse, rat, rabbit, cow, sheep, pig, or primate.

Also disclosed are animals produced by the process of adding to the animal any of the cells disclosed herein.

Disclosed are methods of making a composition capable of inhibiting E7 cellular proliferation activity comprising mixing an E7 inhibiting compound with a pharmaceutically acceptable carrier, wherein the compound is identified, or can be identified, by administering the compound to a system, wherein the system causes maintenance of Akt, assaying the effect of the compound on the amount of Akt in the system, and selecting a compound which causes a decrease in the amount of Akt present in the system.

Disclosed are methods of making a compound that inhibits E7 cellular proliferation activity comprising, a) administering a compound to a system, wherein the system causes maintenance of Akt, b) assaying the effect of the compound on the amount of Akt in the system, c) selecting a compound which causes a decrease in the amount of Akt present in the system, and d) synthesizing the compound.

Disclosed are methods of making a composition capable of inhibiting E7 Akt maintenance activity comprising mixing the compound with a pharmaceutical carrier and wherein the compound is identified, or can be identified, by administering the compound to a system, wherein the system comprises E7 Akt maintenance activity, assaying the effect of the compound on E7 Akt maintenance activity, and selecting a compound which inhibits E7 Akt maintenance activity.

Disclosed are methods of making a compound capable of reversing the effect E7 has on Akt comprising, a) administering a compound to a system, wherein the system comprises E7 Akt maintenance activity, b) assaying the effect of the compound on E7 Akt maintenance activity, c) selecting a compound which inhibits E7 Akt maintenance activity, and d) synthesizing the compound.

It is understood that the disclosed methods can also be performed where the amount of active, i.e. phosphorylated Akt, is present are assayed. It is also understood that the disclosed methods can be performed by looking at the activity of Akt, for example, the phosphorylation of p21^{Cip}.

### D. METHODS OF USING THE COMPOSITIONS

The disclosed compositions can be used in a variety of ways as research tools. For example, the disclosed compositions can be used to identify molecule that modulate the effect E7 has on the cell through E7's modulation of Akt activity.

### 1. Methods of Treatment

The disclosed compositions can be used to treat any disease where uncontrolled cellular proliferation occurs such as cancers, typically cancers where uncontrolled proliferation involves E7 or E7 variants or AKT or AKT variants. Cancers that are associated with HPV can be target cancers. For example, Penile, vaginal, and vulvar cancers are associated with HPVs. A non-limiting list of different types of cancers is as follows: lymphomas (Hodgkins and non-Hodgkins), leukemias, carcinomas, carcinomas of solid tissues, squamous cell carcinomas, adenocarcinomas, sarcomas, gliomas, high grade gliomas, blastomas, neuroblastomas, plasmacytomas, histiocytomas, melanomas, adenomas, hypoxic tumours, myelomas, AIDS-related lymphomas or sarcomas, metastatic cancers, or cancers in general.

A representative but non-limiting list of cancers that the disclosed compositions can be used to treat is the following: lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, kidney cancer, lung cancers such as small cell lung cancer and non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, colon cancer, cervical cancer, cervical carcinoma, breast cancer, and epithelial cancer, renal cancer, genitourinary cancer, pulmonary cancer, esophageal carcinoma, head and neck carcinoma, large bowel cancer, hematopoietic cancers; testicular cancer; colon and rectal cancers, prostatic cancer, or pancreatic cancer.

Compounds disclosed herein may also be used for the treatment of precancer conditions such as cervical and anal dysplasias, other dysplasias, severe dysplasias, hyperplasias, atypical hyperplasias, and neoplasias.

Disclosed are methods of inhibiting aberrant cellular proliferation comprising, administering a compound which inhibits E7 Akt maintenance activity.

Also disclosed are methods of inhibiting abberant cellular proliferation, wherein administering the compound occurs in a subject or wherein the subject is a subject who has cancer.

Also disclosed are methods of inhibiting E7 cellular proliferation activity comprising administering a compound, wherein the compound causes degradation of active Akt, wherein the compound is defined as a compound capable of being identified by administering the compound to a system, wherein the system causes maintenance of active Akt, assaying the effect of the compound on the amount of active Akt in the system, and selecting a compound which causes a decrease in the amount of active Akt present in the system.

Disclosed are methods of inhibiting E7 cellular proliferation activity comprising administering a compound that causes the degradation of active Akt.

Disclosed are methods of inhibiting E7 cellular proliferation activity comprising administering a compound, wherein the compound is identified, or canbe identified, as maintaining Akt activity.

Disclosed are methods of inhibiting E7 cellular proliferation activity comprising administering an inhibitor of E7 Akt maintenance activity, wherein the inhibitor is a compound capable of being identified by administering the compound to a system, wherein the system comprises E7 Akt maintenance activity, assaying the effect of the compound on E7 Akt maintenance activity, and selecting a compound which inhibits E7 Akt maintenance activity.

Disclosed are methods of inhibiting E7 cellular proliferation activity comprising administering an inhibitor of E7 Akt maintenance activity.

Disclosed are methods of inhibiting E7 cellular proliferation activity comprising administering a compound, wherein the compound is identified, or can be identified, as inhibiting E7 Akt maintenance activity.

It is understood that all of the disclosed methods for identifying the various compounds and compositions discussed herein can also represent methods of inhibiting the vartious E7 effects as well as methods of making the various compounds and compositions identified.

It is understood that the disclosed methods can also be performed where the amount of active, i.e. phosphorylated Akt, is present are assayed. It is also understood that the disclosed methods can be performed by looking at the activity of Akt, for example, the phosphorylation of p21^{Cip}.

### 2. Methods of Identifying Modulators of the E7 Affect on Akt and on p21^{Cip}

The disclosed systems can be used to identify compositions that modulate the effect of E7 on Akt, for example.

Disclosed are methods of identifying a compound that inhibits E7 cellular proliferation activity comprising, a) administering a compound to a system, wherein the system causes maintenance of Akt activity; b) assaying the effect of the compound on the amount of Akt activity in the system; and c) selecting a compound which causes a decrease in the amount of Akt activity present in the system.

Disclosed are methods of identifying a compound capable of reversing the effect E7 has on Akt comprising, a) administering a compound to a system, wherein the system comprises E7 active Akt maintenance activity, b) assaying the effect of the compound on E7 active Akt maintenance activity, and c) selecting a compound which inhibits E7 active Akt maintenance activity.

Disclosed are methods of identifying a compound which promotes the nuclear localization of p21^{Cip1} comprising, a) administering a compound to a system, wherein the system comprises E7 p21^{Cip1} cytoplasmic localization activity, b) assaying the effect of the compound on E7 p21^{Cip1} cytoplasmic localization activity, and c) selecting a compound which promotes p21^{Cip1} nuclear localization activity.

Disclosed are methods of promoting p21^{Cip1} nuclear localization, comprising a) administering a compound to a system, wherein the system comprises E7 p21^{Cip1} cytoplasmic localization activity, b) assaying the effect of the compound on E7 p21^{Cip1} cytoplasmic localization activity, and c) selecting a compound which promotes p21^{Cip1} nuclear localization activity.

Disclosed are methods of identifying an inhibitor of an interaction between Akt and E7 comprising a) administering a compound to a system, wherein the system comprises E7, b) assaying the effect of the compound on an E7-Akt interaction, and c) selecting a compound which inhibits E7 Akt interaction.

It is understood that the disclosed methods and systems for identifying molecules having the E7 effect inhibiting properties disclosed herein can be coupled with combinatorial chemistry protocols and concepts. A discussion of combinatorial chemistry and general methods and concepts is discussed herein as well as computer assisted composition design. For example, molecules that are identified using in vitro combinatorial chemistry methods can then be assayed for appropriate activity in the disclosed system. The systems could also for example, be used in an interative screening process using many potential inhibitors as discussed herein.

It is understood that the disclosed methods can also be performed where the amount of active, i.e. phosphorylated Akt, is present are assayed. It is also understood that the disclosed methods can be performed by looking at the activity of Akt, for example, the phosphorylation of p21^{Cip}.

a) Compositions Identified by Screening with Disclosed Compositions/Combinatorial Chemistry

(1) Combinatorial Chemistry

The disclosed compositions and relationships can be used as targets for any combinatorial technique to identify molecules or macromolecular molecules that interact with the disclosed compositions and effect the relationships in a desired way. Also disclosed are the compositions that are identified through combinatorial techniques or screening techniques in which E7 or Akt or portions thereof, for example, are used as the target in a combinatorial or screening protocol.

It is understood that when using the disclosed compositions in combinatorial techniques or screening methods, molecules, such as macromolecular molecules, will be identified that have particular desired properties such as inhibition or stimulation or the target molecule's function. The molecules identified and isolated when using the disclosed compositions, such as, E7 or Akt, for example, or the cells disclosed herein, are also disclosed. Thus, the products produced using the combinatorial or screening approaches that involve the disclosed compositions, such as, E7, Akt, or the cells disclosed are also considered herein disclosed.

Combinatorial chemistry includes but is not limited to all methods for isolating small molecules or macromolecules that are capable of binding either a small molecule or another macromolecule, typically in an iterative process. Proteins, oligonucleotides, and sugars are examples of macromolecules. For example, oligonucleotide molecules with a given function, catalytic or ligand-binding, can be isolated from a complex mixture of random oligonucleotides in what has been referred to as “in vitro genetics” (Szostak, TIBS 19:89, 1992). One synthesizes a large pool of molecules bearing random and defined sequences and subjects that complex mixture, for example, approximately 10¹⁵ individual sequences in 100 μg of a 100 nucleotide RNA, to some selection and enrichment process. Through repeated cycles of affinity chromatography and PCR amplification of the molecules bound to the ligand on the column, Ellington and Szostak (1990) estimated that 1 in 10¹⁰ RNA molecules folded in such a way as to bind a small molecule dyes. DNA molecules with such ligand-binding behavior have been isolated as well (Ellington and Szostak, 1992; Bock et al, 1992). Techniques aimed at similar goals exist for small organic molecules, proteins, antibodies and other macromolecules known to those of skill in the art. Screening sets of molecules for a desired activity whether based on small organic libraries, oligonucleotides, or antibodies is broadly referred to as combinatorial chemistry. Combinatorial techniques are particularly suited for defining binding interactions between molecules and for isolating molecules that have a specific binding activity, often called aptamers when the macromolecules are nucleic acids.

There are a number of methods for isolating proteins which either have de novo activity or a modified activity. For example, phage display libraries have been used to isolate numerous peptides that interact with a specific target. (See for example, U.S. Pat. No. 6,031,071; 5,824,520; 5,596,079; and 5,565,332 which are herein incorporated by reference at least for their material related to phage display and methods relate to combinatorial chemistry)

A preferred method for isolating proteins that have a given function is described by Roberts and Szostak (Roberts R. W. and Szostak J. W. Proc. Natl. Acad. Sci. USA, 94(23)12997-302 (1997). This combinatorial chemistry method couples the functional power of proteins and the genetic power of nucleic acids. An RNA molecule is generated in which a puromycin molecule is covalently attached to the 3′-end of the RNA molecule. An in vitro translation of this modified RNA molecule causes the correct protein, encoded by the RNA to be translated. In addition, because of the attachment of the puromycin, a peptdyl acceptor which cannot be extended, the growing peptide chain is attached to the puromycin which is attached to the RNA. Thus, the protein molecule is attached to the genetic material that encodes it. Normal in vitro selection procedures can now be done to isolate functional peptides. Once the selection procedure for peptide function is complete traditional nucleic acid manipulation procedures are performed to amplify the nucleic acid that codes for the selected functional peptides. After amplification of the genetic material, new RNA is transcribed with puromycin at the 3′-end, new peptide is translated and another functional round of selection is performed. Thus, protein selection can be performed in an iterative manner just like nucleic acid selection techniques. The peptide which is translated is controlled by the sequence of the RNA attached to the puromycin. This sequence can be anything from a random sequence engineered for optimum translation (i.e. no stop codons etc.) or it can be a degenerate sequence of a known RNA molecule to look for improved or altered function of a known peptide. The conditions for nucleic acid amplification and in vitro translation are well known to those of ordinary skill in the art and are preferably performed as in Roberts and Szostak (Roberts R. W. and Szostak J. W. Proc. Natl. Acad. Sci. USA, 94(23)12997-302 (1997)).

Another preferred method for combinatorial methods designed to isolate peptides is described in Cohen et al. (Cohen B.A.,et al., Proc. NatI. Acad. Sci. USA 95(24): 14272-7 (1998)). This method utilizes and modifies two-hybrid technology. Yeast two-hybrid systems are useful for the detection and analysis of protein:protein interactions. The two-hybrid system, initially described in the yeast Saccharomyces cerevisiae, is a powerful molecular genetic technique for identifying new regulatory molecules, specific to the protein of interest (Fields and Song, Nature 340:245-6 (1989)). Cohen et al., modified this technology so that novel interactions between synthetic or engineered peptide sequences could be identified which bind a molecule of choice. The benefit of this type of technology is that the selection is done in an intracellular environment. The method utilizes a library of peptide molecules that attached to an acidic activation domain. A peptide of choice, for example a portion of AKT OR E7 is attached to a DNA binding domain of a transcriptional activation protein, such as Gal 4. By performing the Two-hybrid technique on this type of system, molecules that bind the portion of Akt or E7 can be identified.

Using methodology well known to those of skill in the art, in combination with various combinatorial libraries, one can isolate and characterize those small molecules or macromolecules, which bind to or interact with the desired target. The relative binding affinity of these compounds can be compared and optimum compounds identified using competitive binding studies, which are well known to those of skill in the art.

Techniques for making combinatorial libraries and screening combinatorial libraries to isolate molecules which bind a desired target are well known to those of skill in the art. Representative techniques and methods can be found in but are not limited to U.S. Pat. Nos. 5,084,824, 5,288,514, 5,449,754, 5,506,337, 5,539,083, 5,545,568, 5,556,762, 5,565,324, 5,565,332, 5,573,905, 5,618,825, 5,619,680, 5,627,210, 5,646,285, 5,663,046, 5,670,326, 5,677,195, 5,683,899, 5,688,696, 5,688,997, 5,698,685, 5,712,146, 5,721,099, 5,723,598,5,741,713, 5,792,431, 5,807,683, 5,807,754, 5,821,130, 5,831,014, 5,834,195, 5,834,318,5,834,588, 5,840,500, 5,847,150, 5,856,107, 5,856,496, 5,859,190, 5,864,010, 5,874,443, 5,877,214, 5,880,972, 5,886,126, 5,886,127, 5,891,737, 5,916,899, 5,919,955, 5,925,527, 5,939,268, 5,942,387, 5,945,070, 5,948,696, 5,958,702, 5,958,792, 5,962,337, 5,965,719,5,972,719, 5,976,894, 5,980,704, 5,985,356, 5,999,086, 6,001,579, 6,004,617, 6,008,321, 6,017,768, 6,025,371, 6,030,917, 6,040,193, 6,045,671, 6,045,755, 6,060,596, and 6,061,636.

Combinatorial libraries can be made from a wide array of molecules using a number of different synthetic techniques. For example, libraries containing fused 2,4-pyrimidinediones (U.S. Pat. No. 6,025,371) dihydrobenzopyrans (U.S. Pat. Nos. 6,017,768and 5,821,130), amide alcohols (U.S. Pat. No. 5,976,894), hydroxy-amino acid amides (U.S. Pat. No. 5,972,719) carbohydrates (U.S. Pat. No. 5,965,719), 1,4-benzodiazepin-2,5-diones (U.S. Pat. No. 5,962,337), cyclics (U.S. Pat. No. 5,958,792), biaryl amino acid amides (U.S. Pat. No. 5,948,696), thiophenes (U.S. Pat. No. 5,942,387), tricyclic Tetrahydroquinolines (U.S. Pat. No. 5,925,527), benzofurans (U.S. Pat. No. 5,919,955), isoquinolines (U.S. Pat. No. 5,916,899), hydantoin and thiohydantoin (U.S. Pat. No. 5,859,190), indoles (U.S. Pat. No. 5,856,496), imidazol-pyrido-indole and imidazol-pyrido-benzothiophenes (U.S. Pat. No. 5,856,107) substituted 2-methylene-2,3-dihydrothiazoles (U.S. Pat. No. 5,847,150), quinolines (U.S. Pat. No. 5,840,500), PNA (U.S. Pat. No. 5,831,014), containing tags (U.S. Pat. No. 5,721,099), polyketides (U.S. Pat. No. 5,712,146), morpholino-subunits (U.S. Pat. Nos. 5,698,685 and 5,506,337), sulfamides (U.S. Pat. No. 5,618,825), and benzodiazepines (U.S. Pat. No. 5,288,514).

As used herein combinatorial methods and libraries included traditional screening methods and libraries as well as methods and libraries used in interative processes.

(2) Computer Assisted Drug Design

The disclosed compositions can be used as targets for any molecular modeling technique to identify either the structure of the disclosed compositions or to identify potential or actual molecules, such as small molecules, which interact in a desired way with the disclosed compositions.

It is understood that when using the disclosed compositions in modeling techniques, molecules, such as macromolecular molecules, will be identified that have particular desired properties such as inhibition or stimulation or the target molecule's function. The molecules identified and isolated when using the disclosed compositions, such as, E7 or Akt or the cells disclosed herein, are also disclosed. Thus, the products produced using the combinatorial or screening approaches that involve the disclosed compositions, such as, E7, Akt, or the cells disclosed are also considered herein disclosed.

Thus, one way to isolate molecules that bind a molecule of choice is through rational design. This is achieved through structural information and computer modeling. Computer modeling technology allows visualization of the three-dimensional atomic structure of a selected molecule and the rational design of new compounds that will interact with the molecule. The three-dimensional construct typically depends on data from x-ray crystallographic analyses or NMR imaging of the selected molecule. The molecular dynamics require force field data. The computer graphics systems enable prediction of how a new compound will link to the target molecule and allow experimental manipulation of the structures of the compound and target molecule to perfect binding specificity. Prediction of what the molecule-compound interaction will be when small changes are made in one or both requires molecular mechanics software and computationally intensive computers, usually coupled with user-friendly, menu-driven interfaces between the molecular design program and the user.

Examples of molecular modeling systems are the CHARMm and QUANTA programs, Polygen Corporation, Waltham, Mass. CHARMm performs the energy minimization and molecular dynamics functions. QUANTA performs the construction, graphic modeling and analysis of molecular structure. QUANTA allows interactive construction, modification, visualization, and analysis of the behavior of molecules with each other.

A number of articles review computer modeling of drugs interactive with specific proteins, such as Rotivinen, et al., 1988 Acda Pharmaceutica Fennica 97, 159-166; Ripka, New Scientist 54-57 (Jun. 16, 1988); McKinaly and Rossmann, 1989 Annu. Rev. Pharinacol._Toxiciol. 29, 111-122; Perry and Davies, QSAR: Ouantitative Structure-Activity Relationships in Drug Design pp. 189-193 (Alan R. Liss, Inc. 1989); Lewis and Dean, 1989 Proc. R. Soc. Lond. 236, 125-140 and 141-1 62; and, with respect to a model enzyme for nucleic acid components, Askew, et al., 1989 J. Am. Chem. Soc. 111, 1082-1090. Other computer programs that screen and graphically depict chemicals are available from companies such as BioDesign, Inc., Pasadena, Calif., Allelix, Inc, Mississauga, Ontario, Canada, and Hypercube, Inc., Cambridge, Ontario. Although these are primarily designed for application to drugs specific to particular proteins, they can be adapted to design of molecules specifically interacting with specific regions of DNA or RNA, once that region is identified.

Although described above with reference to design and generation of compounds which could alter binding, one could also screen libraries of known compounds, including natural products or synthetic chemicals, and biologically active materials, including proteins, for compounds which alter substrate binding or enzymatic activity.

### E. EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in IC or is at ambient temperature, and pressure is at or near atmospheric.

### 1. Example 1

a) Results

(1) E7 Overcomes Raf-Induced Growth Arrest.

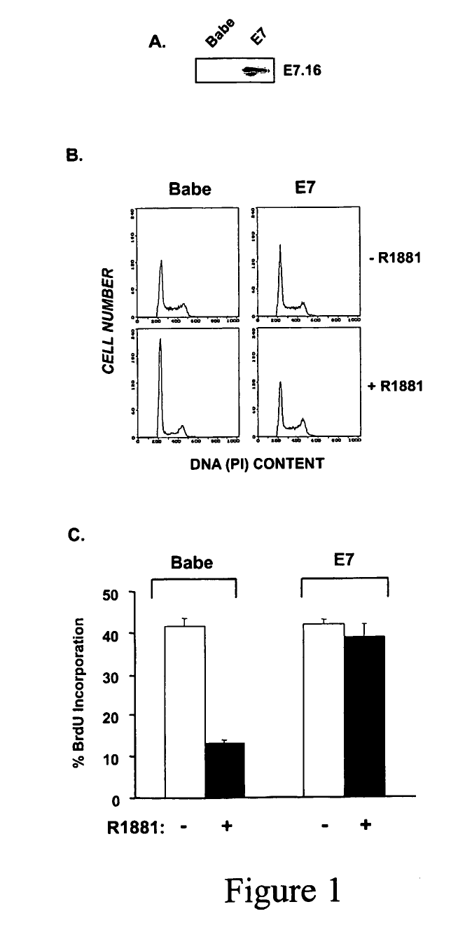

The ability of HPV16 E7 to abrogate p21^{Cip1}-dependent growth arrest has been observed, but the targets of E7 to propogate this effect are disclosed herein. Activation of the Ras/Raf/MAPK pathway induces cell cycle arrest in various primary and immortal cell types (Hirakawa and Ruley, 1988; Lloyd et al., 1997; Ridley et al., 1988; Serrano et al., 1997; Sewing et al., 1997; Woods et al., 1997). Importantly, biochemical and genetic approaches indicate that p21^{Cip1} is an essential mediator of this arrest phenotype. To explore the molecular basis for E7-mediated bypass of Ras-induced arrest, a conditional RafAR molecule in which an activated Raf kinase has been fused to the androgen receptor hormone-binding domain was used (Sewing, A., B. Wiseman, A. C. Lloyd, and H. Land 1997. High-intensity Raf signal causes cell cycle arrest mediated by p21Cip1 Molecular & Cellular Biology. 17:5588-97). Importantly, Raf acts directly downstream of Ras ( Moodie, S. A., B. M. Willumsen, M. J. Weber, and A. Wolfman 1993. Complexes of Ras.GTP with Raf-1 and mitogen-activated protein kinase kinase Science. 260:1658-61, Vojtek, A. B., S. M. Hollenberg, and J. A. Cooper 1993. Mammalian Ras interacts directly with the serine/threonine kinase Raf Cell. 74:205-14.), and Ras effector loop mutants that preferentially activate Raf (but not PI(3)K or Ral.GDS) confer growth arrest similar to oncogenic Ras (Lin, A. W., M. Barradas, J. C. Stone, L. van Aelst, M. Serrano, and S. W. Lowe 1998. Premature senescence involving p53 and p16 is activated in response to constitutive MEK/MAPK mitogenic signaling Genes & Development. 12:3008-19.). Additionally, Raf renders similar growth and morphological phenotypes as Ras in primary and immortal fibroblasts (Lin, A. W., M. Barradas, J. C. Stone, L. van Aelst, M. Serrano, and S. W. Lowe 1998. Premature senescence involving p53 and p16 is activated in response to constitutive MEK/MAPK mitogenic signaling Genes & Development. 12:3008-19., Sewing, A., B. Wiseman, A. C. Lloyd, and H. Land 1997. High-intensity Raf signal causes cell cycle arrest mediated by p21Cip1 Molecular & Cellular Biology. 17:5588-97). In NIH3T3 fibroblasts, activation of a steroid hormone regulatable RafAR kinase leads to inhibition of DNA synthesis preceded by increased expression of p21^{Cip1} and loss of cyclin E-CDK2 kinase activity (Sewing et al., 1997). As such, this cell-based system represents a relevant context within which to examine the interplay between E7 and p21^{Cip1}. In order to assess the effects of E7 in this system, RafAR-expressing NIH3T3 were infected with the retroviral vector pBabe (Morgenstem and Land, 1990) or its derivative encoding HPV16 E7. Infected cells were pooled, examined for E7 expression (FIG. 1A), and used for subsequent experiments. Activation of RafAR with 1.0 μM R1881 led to morphological changes including elongation and development of extended processes (data not shown). These RafAR-induced alterations in cell morphology, which are consistent with previous descriptions in NIH3T3 and other cell types (Lloyd et al., 1997; Sewing et al., 1997), were not affected in E7-expressing cells, indicating that at least some components of Raf signaling are not disrupted by the presence of E7. Upon examination of cell proliferation, RafAR activation led to G1 arrest in control cells (Babe), with >85% of cells accumulating in G1 (FIG. 1B). This was accompanied by inhibition of DNA synthesis as shown in FIG. 1C. However, cells expressing E7 continued cell cycle progression (FIG. 1B) and DNA synthesis (FIG. 1C) in the presence of activated RafAR. Similar observations have been made in separately generated clonal and pooled E7-expressing cell lines. These results suggest that E7 perturbs Raf-induced negative regulation at the G1-S transition and are in agreement with previous observations that E7 transforms cells in cooperation with an activated Ras/Raf pathway (Halpem, 1997).

(2) E7 Prevents Inactivation of Cyclin E-CDK2 by p21^{Cip1}

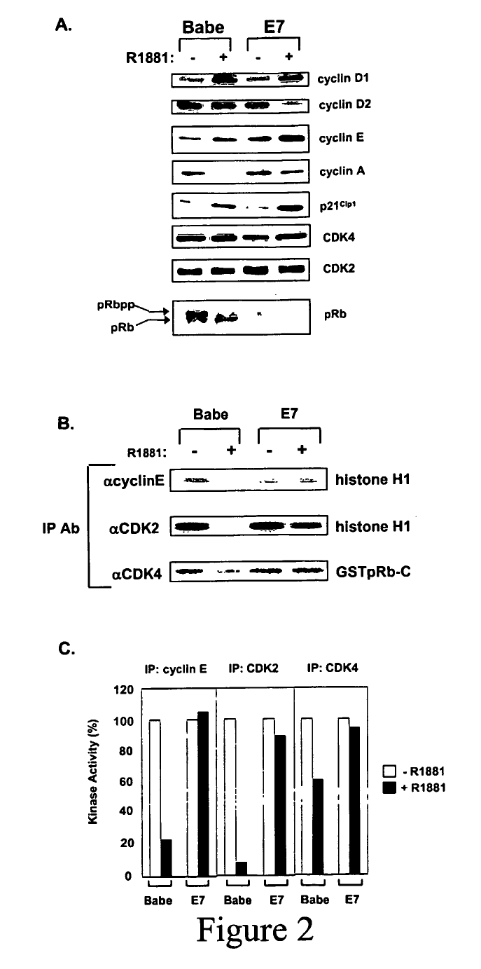

In NIH3T3 fibroblasts, Raf activation leads to inhibition of DNA synthesis preceded by increased expression of p21^{Cip1} and loss of cyclin E-CDK2 kinase activity (Sewing, A., B. Wiseman, A. C. Lloyd, and H. Land 1997. High-intensity Raf signal causes cell cycle arrest mediated by p21Cip1 Molecular & Cellular Biology. 17:5588-97, Woods, D., D. Parry, H. Cherwinski, E. Bosch, E. Lees, and M. McMahon 1997. Raf-induced proliferation or cell cycle arrest is determined by the level of Raf activity with arrest mediated by p21Cip1 Molecular & Cellular Biology. 17:5598-611.). To detennine the nature of resistance to RafAR-induced arrest in E7-expressing cells, the expression levels and activities of G 1-specific cyclins, CDKs, and CKIs were examined. Activation of RafAR led to induction of cyclin D1 and cyclin E in control and E7-expressing cells (FIG. 2A). Importantly, p21^{Cip1} was also elevated in both cell lines upon RafAR stimulation (FIG. 2A). Consistent with observations in other p21^{Cip1}-dependent arrest systems (Funk et al., 1997; Jones et al., 1997; Ruesch and Laimins, 1997), this implies that E7 does not overcome p21^{Cip1}-mediated arrest by preventing p21^{Cip1} expression. In RafAR-arrested control cells, stimulation of RafAR resulted in the loss of steady state cyclin A and accumulation of hypophosphorylated RB (FIG. 2A). Cyclin E-CDK2 activity is required for hyperphosphorylation of RB and cyclin A expression (Rudolph et al., 1996; Weinberg, 1995; Zerfass-Thome et al., 1997). In accordance, control cells exhibited significant loss of cyclin E-CDK2 activity and a less dramatic decrease in CDK4-associated kinase activity (FIG. 2B and FIG. 2C), likely due to a greater sensitivity of CDK2 to inhibition by Kip/Cip CKIs (Cheng et al., 1999; LaBaer et al., 1997; Polyak et al., 1994b). In contrast, cyclin E-CDK2 and CDK4 kinase activities were maintained upon RafAR stimulation of E7-expressing cells (FIG. 2B and FIG. 2C). In addition, cyclin A expression and RB hyperphosphorylation were similar in asynchronous and RafAR-activated cells in the presence of E7. Taken together, these results indicate that E7 overcomes RafAR-induced arrest by abrogating the CDK2-inhibitory function of p21^{Cip1}.

(3) E7 Does Not Derepress p21Cip1-Associated CDK2 Activity

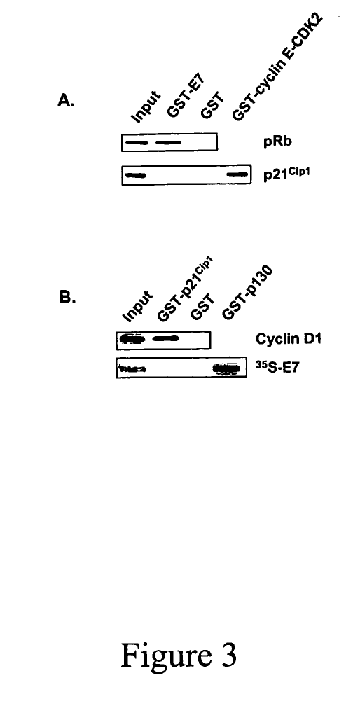

In response to observations that E7 maintains CDK2 activity in the context of p21^{Cip1}-mediated arrest, a model has been proposed that p21^{Cip1}-associated CDK2 complexes are derepressed via the p21^{Cip1}-E7 interaction (Funk et al., 1997). However, others have reported that E7 does not associate with p21^{Cip1} (Hickman, E. S., S. Bates, and K. H. Vousden 1997. Pertubation of the p53 response by human papillomavirus type 16 E7 Journal of Virology. 71:3710-3718., Ruesch, M., and L. A. Laimins 1997. Initiation of DNA synthesis by human papillomavirus E7 oncoproteins is resistant to p21 mediated inhibition of cyclin E-cdk2 activity Journal of Virology. 71:5570-5578.). This model was examined within the context of the E7-RafAR system by testing two key predictions: (1) E7 should interact with p21^{Cip1} in cell lysates, and (2) p21^{Cip1} should be associated with active cyclin E-CDK2 complexes. Standard coimrnmunoprecipitations with E7- and p21^{Cip1}-specific antibodies were used. No interaction between E7 and p21^{Cip1} was detected. However, E7 expression is low in this system, raising the possibility that the putative E7- p21^{Cip1} interaction was not detected due to technical limitations. To confirm the coimmunoprecipitation results, purified recombinant GST-E7 was mixed with control cell lysates and precipitated complexes were examined for the presence of p21^{Cip1} by Western blot analysis. p21^{Cip1} was not found in GST-E7 precipitates (FIG. 3A). As controls, p21^{Cip1} was detected in GST-cyclin E-CDK2 precipitates, and RB was associated with GST-E7. In the reciprocal experiment, GST-p21^{Cip1} was mixed with cell lysate and an excess of radiolabelled E7. No p21^{Cip1}-E7 interaction was observed, while GST-p130/³⁵S-E7 and GST-p21^{Cip1}/cyclin D1 complexes were detected (FIG. 3B). These results indicate that E7 does not associate with p21^{Cip1} in this system.

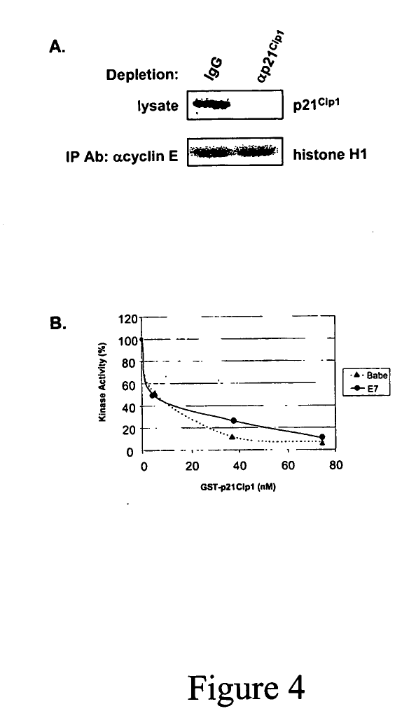

In order to address the second corollary that p21^{Cip1} is associated with active cyclin E-CDK2 in E7-expressing cells, an immunodepletion approach was used. Depletion of p21^{Cip1}-containing complexes from cell lysates did not reduce the level of remaining cyclin E-CDK2 activity. RafAR-induced E7-expressing cell lysates were subjected to three rounds of immunodepletion with control or p21^{Cip1}-specific antibodies shown previously to precipitate all known cyclin-CDK-p21^{Cip1} complexes (Cai and Dynlacht, 1998). p21^{Cip1} was efficiently depleted as monitored by western blot analysis (FIG. 4A). Cyclin E-CDK2 complexes were then immunoprecipitated from depleted lysates and assessed for kinase activity. p21^{Cip1}-specific immunodepletion reduced p21^{Cip1} levels by >95%, but did not alter the residual cyclin E-CDK2 kinase activity (FIG. 4A), indicating that any depleted p21^{Cpi1}-cyclinE-CDK2 complexes were inactive and that E7 does not derepress p21^{Cip1}-associated CDK2. In addition, cyclin E and CDK2 are not rendered intrinsically resistant to p21^{Cip1} by E7, as cyclin E-associated kinase activity in lysates of control and E7-expressing cells was equally sensitive to inhibition by purified recombinant GST-p21^{Cip1} (FIG. 4B).

(4) Enhanced Expression of Cyclin E Does Not Overcome RafAR-Induced Arrest

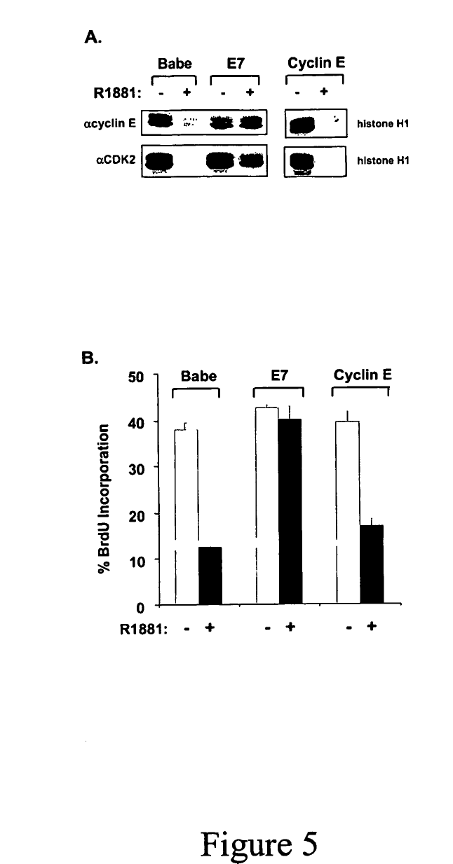

Expression of E7 increased cyclin E protein levels approximately 2.5-3 fold (FIG. 2A), which is consistent with the ability of E7 to dysregulate RB-E2F transcriptional regulation of the cyclin E gene (Funk et al., 1997; Zerfass et al., 1995). This effect is independent from, and additive to, the RafAR-induced elevation of cyclin E (FIG. 2A). Since E7 did not alter the intrinsic sensitivity of cyclin E-CDK2 to p21^{Cip1}, whether dysregulation of cyclin E expression by E7 may be sufficient to overcome RafAR-induced, p21^{Cip1}-mediated arrest was addessed. This scenario was examined by stable expression of human cyclin E in RafAR-NIH3T3. Exogenous human cyclin E associates with and activates endogenous murine CDK2 (Alevizopoulos et al., 1997; Vlach et al., 1996), as human cyclin E-specific antisera precipitated robust levels of kinase activity (FIG. 5A, top panel). However, retroviral expression of cyclin E did not abrogate RafAR-induced arrest (FIG. 5B). In addition, immunoprecipitation of human cyclin E complexes or total endogenous CDK2 revealed that exogenous cyclin E expression did not prevent p21^{Cip1}-mediated inhibition of cyclin E-CDK2 (FIG. 5A), suggesting that the activity of E7 in this system is not defined solely by induction of cyclin E levels. These observations are consistent with other reports that arrest imposed by Kip/Cip CKIs cannot be overcome by elevated physiological accumulation of cyclin E (Alevizopoulos et al., 1997; Perez-Roger et al., 1997).

(5) E7 Alters the Stoichiometry Between p21^{Cip1} and Cyclin E-CDK2

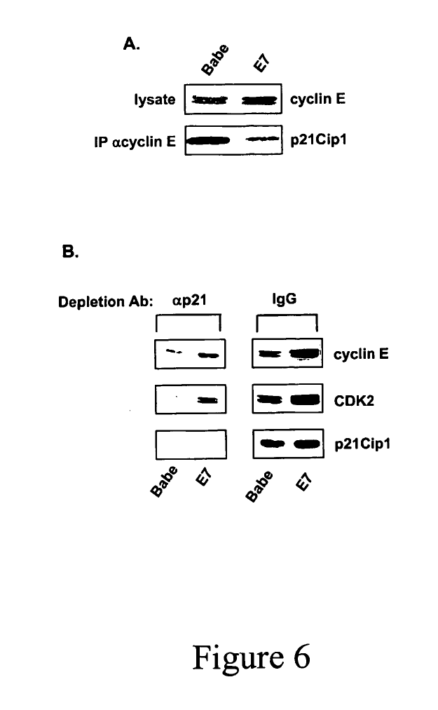

Upon RafAR-activation, cells expressing E7 maintained cyclin E-CD:K2 activity (FIG. 2A) in complexes free of p21^{Cip1} (FIG. 4A), suggesting that E7 may prevent the association of p21^{Cpi1} with a pool of cyclin E-CDK2 complexes. Equal amounts of p21^{Cpi1} were associated with cyclin E-immunoprecipitates in RafAR-induced control and E7-expressing cells (data not shown). However, since there is a significant increase of cyclin E steady state levels in E7-expressing cells (FIG. 2A), this observation would imply that the stoichiometry of p21^{Cip1} and cyclin E-CDK2 is altered in the presence of E7. To illustrate this contention more clearly, cyclin E-containing complexes were immunoprecipitated from RafAR-induced control or E7 cell lysate, standardizing on the level of cyclin E expression (FIG. 6A, top panel). As seen in FIG. 6A (bottom panel), cyclin E-associated p21^{Cip1} was significantly lower in E7-expressing cells. This suggests that E7 expression may lead to accumulation of p21^{Cip1}-free cyclin E-CDK2 complexes. In order to examine this hypothesis more directly, RafAR-induced control or E7 cell lysates were depleted with p21^{Cip1}-specific antisera as described in FIG. 4A. Mock or p21^{Cip1}-depleted lysates were subsequently analyzed for remaining cyclin E, CDK2, and p21^{Cip1}. The levels of cyclin E and CDK2 were significantly reduced by the p21^{Cip1}-specific antisera in both cell types (FIG. 6B, left panel). This indicates that a substantial quantity of CDK2 complexes is associated with p21^{Cip1}, consistent with previous reports (Cai and Dynlacht, 1998; Zhang et al., 1994a; Zhang et al., 1994b). However, E7-expressing cell lysates retained approximately 3-fold more cyclin E and CDK2 than the control counterparts in p21^{Cip1}-depleted lysates. Since data from FIG. 4A demonstrates that cyclin E-associated kinase activity is not associated with p21^{Cip1}, the increased pool of p21^{Cip1}-free cyclin E-CDK2 in E7 cells (FIG. 6B) is likely responsible for E7-specific maintenance of cyclin E-CDK2 activity during RafAR activation (FIG. 2B and FIG. 2C). Altogether, these observations indicate that the presence of E7 hinders p21^{Cip1} association with and inhibition of cyclin E-CDK2 complexes in RafAR-activated cells.

(6) E7 Prevents Raf-Induced p21^{Cip1} Nuclear Accumulation

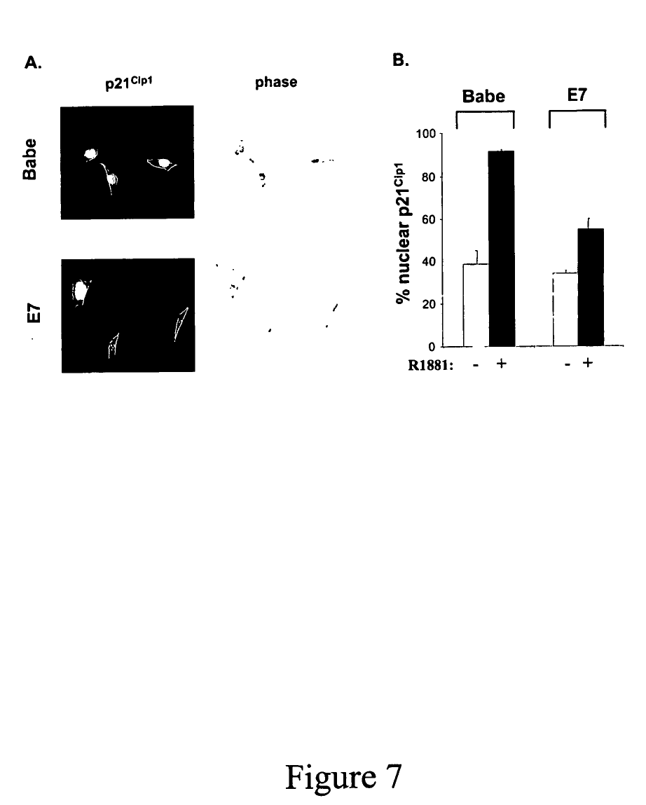

Induction of p21^{Cip1} expression by p53 or other antimitogenic stimuli is accompanied by its nuclear accumulation. Since the localization of p21^{Cip1} is considered to be important in its function as an inhibitor of proliferation (Goubin and Ducommun, 1995; Sherr and Roberts, 1995), the effects of RafAR on p21^{Cip1} cellular localization were examined. Upon staining with p21^{Cip1}-specific antibodies, a similar fraction (˜40%) of control and E7-expressing cells exhibited strong nuclear fluorescence (FIG. 7). This observation is consistent with reports that p21^{Cip1} localizes to the nucleus during mid-G1 (Dulic et al., 1998) as 50-55% of asynchronous cells were in G1 (FIG. 1B). Interestingly, induction of RafAR resulted in a dramatic increase in p21^{Cip1} nuclear accumulation, with >90% of control cells showing nuclear p21^{Cip1}-staining (FIG. 7). This observation suggests that Raf signaling activates p21^{Cip1} function by regulating its cellular localization as well as increasing its synthesis. Notably, E7 expression markedly reduced the RafAR-specific nuclear localization of p21^{Cip1} (FIG. 7), without altering induction of p21^{Cip1} expression (FIG. 2A). As cyclin E is primarily a nuclear protein (Ohtsubo et al., 1995), these results are consistent with the idea that E7 prevents p21^{Cip1} association with cyclin E-CDK2 by inhibiting RafAR-specific nuclear compartmentalization of p21^{Cip1}.

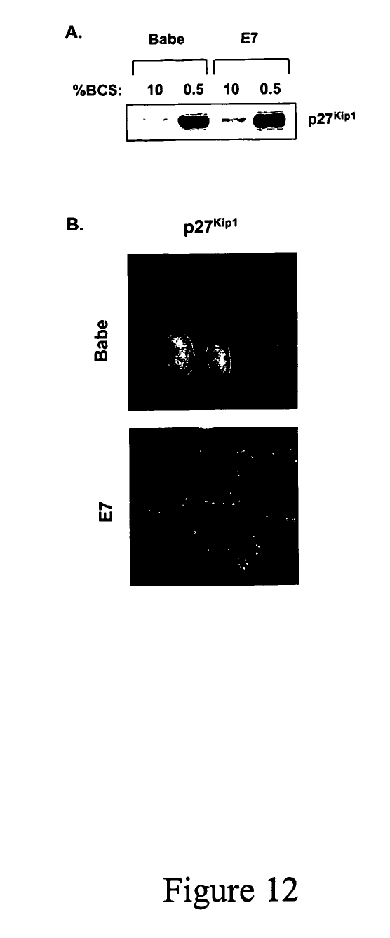

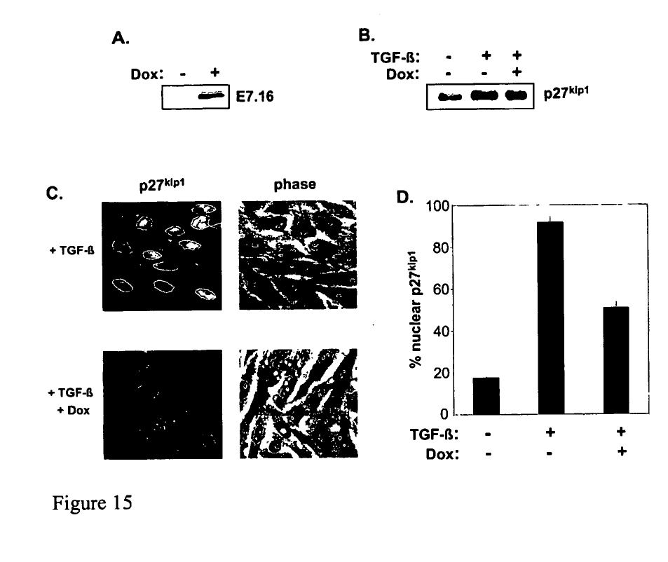

Since E7 has also been shown to abrogate the CKI function of the related p27^{Kip1} during cellular arrest (Schulze, A., B. Mannhardt, K. Zerfass-Thome, W. Zwerschke, and P. Jansen-Durr 1998. Anchorage-independent transcription of the cyclin A gene induced by the E7 oncoprotein of human papillomavirus type 16 Journal of Virology. 72:2323-34.) (data not shown), it was examined whether mislocalization was a more general strategy by which E7 impinges on CKI function. TGF-β signaling leads to p27^{Kip1}-mediated inhibition of cyclin E-CDK2 and cell cycle arrest in the epithelial cell line Mv1Lu (Reynisdottir, I., K. Polyak, A. lavarone, and J. Massague 1995. Kip/Cip and Ink4 Cdk inhibitors cooperate to induce cell cycle arrest in response to TGF-beta Genes & Development. 9:1831-45.). It was investigated whether the effects of E7 on p27^{Kip1} localization in the context of TGF-β utilizing an Mv1Lu derivative that expresses E7 in response to doxycycline (FIG. 15A). Consistent with previous observations (Demers, G. W., E. Espling, J. B. Harry, B. G. Etscheid, and D. A. Galloway 1996. Abrogation of growth arrest signals by human papillomavirus type 16 E7 is mediated by sequences required for transformation Journal of Virology. 70:6862-9.), E7 prevented the arrest imposed by TGF-β (data not shown). Interestingly, TGF-β induced a robust nuclear accumulation of p27^{Kip1} that was alleviated in the presence of E7 (FIG. 15C and FIG. 15D). E7 altered p27^{Kip1} localization without affecting expression levels of the CKI (FIG. 15B). It was also observed this mislocalization of p27^{Kip1} by E7 during growth factor deprivation in fibroblasts (FIG. 12). These results suggest that E7 hinders Kip/Cip CKI function through a conserved mechanism (mislocalization) and in response to multiple antimitogenic signals.

(7) The PI-3K/Akt Pathway is Required for E7-Mediated Abrogation of RafAR-Induced Arrest

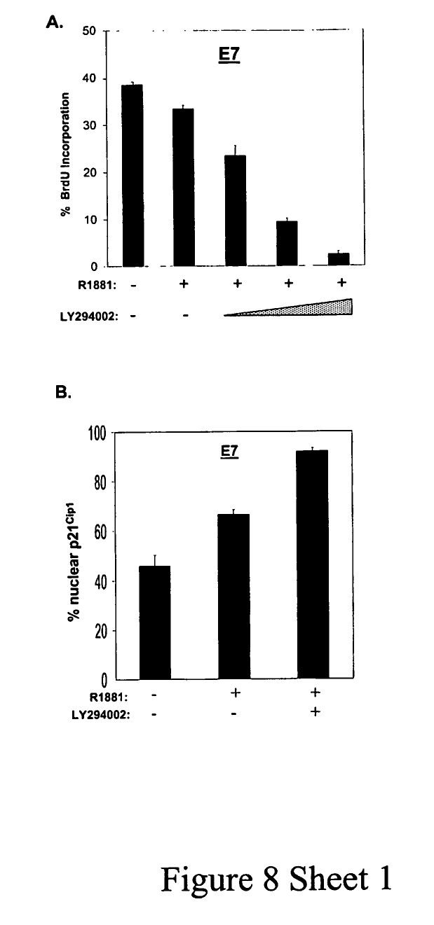

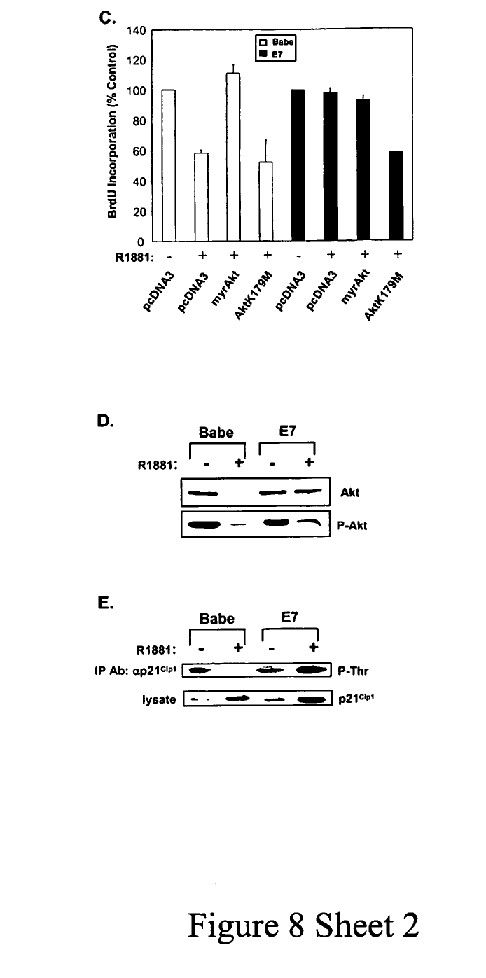

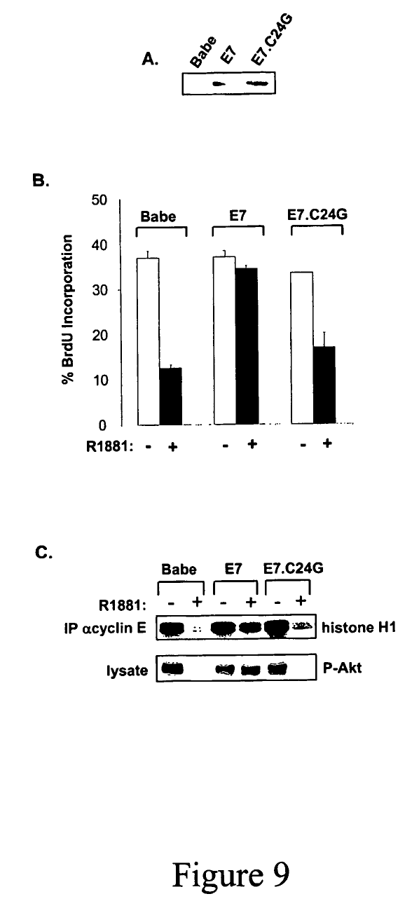

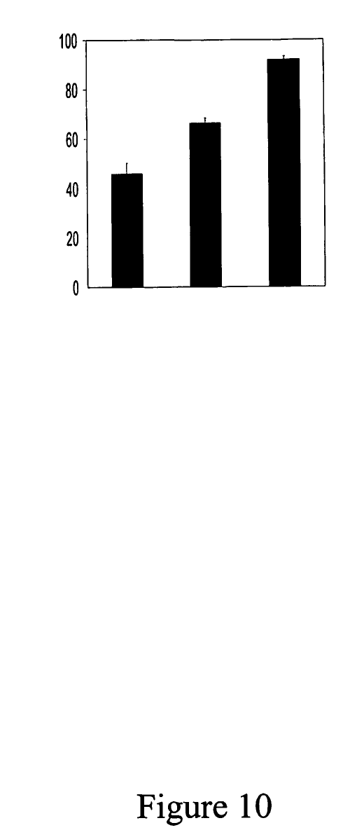

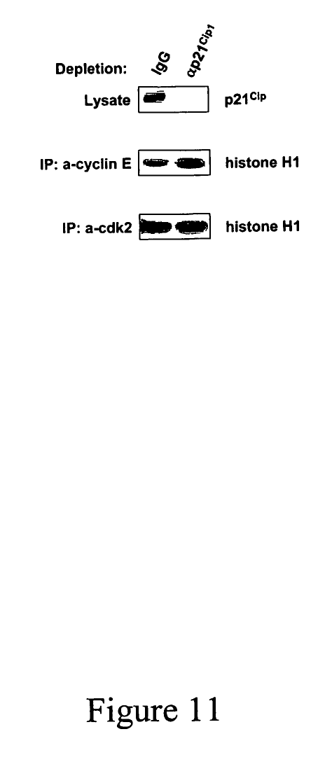

p21^{Cip1} contains a bipartite nuclear localization sequence (NLS) in its C-terminus (Goubin and Ducommun, 1995). Mutation of the NLS reduces the capacity of p21^{Cip1} to inhibit CDK activity and cellular proliferation (Rossig et al., 2001; Sherr and Roberts, 1995; Zhou et al.,2001). Recently, Akt has been shown to phosphorylate threonine-145 within the p21^{Cip1} NLS, leading to cytoplasmic localization of p21^{Cip1} (Zhou et al., 2001). In accordance, inhibition of Akt results in reduced p21^{Cip1} phosphorylation, promoting nuclear accumulation of p21^{Cip1} and growth arrest. In order to assess the role of Akt in E7-mediated abrogation of RafAR-induced arrest, RafAR was activated in E7-expressing cells in the presence or absence of LY294002, an inhibitor of the Akt-activator P13-K. As previously demonstrated, E7-expressing cells continue through the G1-S transition following RafAR activation. However, cell cycle progression of RafAR-induced, E7-expressing cells was diminished in the presence of LY294002 (FIG. 8A). Incubation with LY294002 also restored Raf-induced nuclear accumulation of p21^{Cip1} (FIG. 8B), suggesting that PI3-K/Akt activity is required for E7 to impair p21^{Cip1} localization and overcome RafAR-induced G1 arrest. Because inhibition of PI3-K could have pleiotropic effects beyond the specific activity of Akt, the role of Akt in this system was further shown by utilizing a dominant-negative mutant of Akt, Akt K179M. RafAR-activated control or E7-expressing cells were transiently transfected with vector or Akt K179M expression plasmids in conjunction with a plasmid encoding the green fluorescent protein (GFP). DNA synthesis of the transfected, GFP-positive cells was measured by BrdU-incorporation. As shown in FIG. 8C, introduction of Akt K179M reduced BrdU incorporation of RafAR-induced E7-expressing cells to a level similar to RafAR-induced control cells, indicating that Akt activity is required for E7-mediated bypass of RafAR-arrest. In addition, Akt activity appears sufficient to rescue G1-S progression during RafAR-signaling, since transfection of RafAR-activated control cells with a myristoylated, constitutively active form of Akt (myrAkt) restored BrdU incorporation to asynchronous levels (FIG. 8C). These results suggest that Akt antagonizes Raf-induced arrest. Consequently, we examined the effects of RafAR signaling on the status of Akt using antibodies which detect total or serine-473 phosphorylated (active) Akt. In control cells, the steady state levels of total and active Akt were decreased upon RafAR-activation by 66% and 79%, respectively (FIG. 8D). This RafAR-induced reduction in Akt activity correlated with a significant decrease in threonine-phosphorylated p21^{Cip1} despite elevated levels of total p21^{Cip1} (FIG. 8E). In contrast, E7-expressing cells maintained total and activated Akt at or near asynchronous levels and exhibited a modest increase in threonine-phosphorylated p21^{Cip1} upon RafAR activation (FIG. 8D and FIG. 8E). Taken together, these observations suggest that RafAR signaling may converge on p21^{Cip1} in two ways: inducing transcription of p21^{Cip1} and stimulating p21^{Cip1} nuclear accumulation via negative regulation of Akt. While E7 does not interfere with RafAR-specific expression of p21^{Cip1}, the ability of RafAR to induce Akt down-regulation and p21^{Cip1} nuclear localization is prevented by E7, consistent with the idea that maintenance of Akt is important in E7-mediated cell cycle progression in the presence of RafAR-activation. This is further supported by analysis of the E7.C24G point mutation within the context of the RafAR system. Residue 24 resides in the LXCXE motif of E7 and is essential for the E7-RB interaction (Barbosa et al. EMBO Journal. 9:153-60 (1990), Munger et al. EMBO Journal. 8:4099-105 (1989). Importantly, it has been demonstrated that mutation of the LXCXE motif disrupts the ability of E7 to cooperate with an activated Ras pathway in cellular transformation (Banks et al. Oncogene. 5:1383-1389 (1990), Edmonds et al. Virology. 63:2650-6 (1989), Phelps Journal of Virology. 66:2418-27 (1992)). As represented in FIG. 9B, RafAR-NIH3T3 cells stably expressing E7.C24G were as sensitive to RafAR-induced inhibition of DNA synthesis as cells transduced with empty retrovirus, although the E7- and E7.C24G-expressing cell lines expressed comparable levels of E7 as determined by Western blot analysis (FIG. 9A). This suggests that the LXCXE motif is essential in E7-mediation abrogation of RafAR-induced arrest. In contrast to cells expressing wild-type E7, E7.C24G-expressing cells exhibited RafAR-induced loss of cyclin E-associated kinase activity and were incapable of maintaining active Akt upon RafAR induction (FIG. 9C). Altogether, these observations indicate that persistence of Akt activity may play a role in the ability of E7 to abrogate p21^{Cip1}, function and RafAR-induced arrest. That E7 disrupts p21^{Cip1}-function without derepressing p21^{Cip1}-cyclin-CDK2 complexes is relevant in multiple systems, including the host cell type of HPV.

RafAR-activation led to a similar increase in p21^{Cip1} steady state levels in control and E7-expressing cells. However, the stoichiometry of cyclin E-associated p21^{Cip1} was lower in cells expressing E7 (FIG. 6). The reduced association between p21^{Cip1} and cyclin E-CDK2 in the presence of E7 correlated with an increased pool of p21^{Cip1}-free cyclin E-CDK2. As active cyclin E-CDK2 was not associated with p21^{Cip1} (FIG. 4), this elevation in inhibitor-free cyclin E-CDK2 complexes is likely responsible for the E7-specific maintenance of cyclin E-CDK2 activity during Raf signaling. E7-expressing cells also exhibited increased levels of steady-state cyclin E (FIG. 2), consistent with observations that E7 disrupts E2F-RB-mediated transcriptional regulation of the cyclin E gene (Martin et al., 1998; Zerfass et al., 1995). Although exogenous expression of cyclin E alone was insufficient to maintain cyclin E-CDK2 activity and cell cycle progression upon RafAR-activation (FIG. 5), E7-induced elevation in cyclin E synthesis may contribute to a net gain in p21^{Cip1}-free cyclin E-CDK2. Observations have been made with regard to the inyc proto-oncogene, where activation of Myc restores cyclin E-CDK2 activity in the presence of elevated p21^{Cip1} or the closely related p27^{Kip1} (Bouchard et al., 1999; Perez-Roger et al., 1999; Perez-Roger et al., 1997; Rudolph et al., 1996; Steiner et al., 1995; Vlach et al., 1996). Myc-induced cyclin E-CDK2 activity results from increased cyclin E and cyclin D1/D2 expression, with cyclin D-CDK complexes sequestering Kip/Cip CKIs and cyclin E feeding a CKI-free pool of activatable cyclin E-CDK2 (Bouchard et al., 1999; Perez-Roger et al., 1999). As cyclins D1 and D2 are not upregulated upon E7 expression (FIG. 2), sequestration of p21^{Cip1} by cyclin D-CDK complexes does not likely account for the maintenance of cyclin E-CDK2 activity in the RafAR-E7 system.

The results disclosed here establish a bi-directional antagonism between these components of the Raf/MAPK and PI3-K/Akt signaling pathways. E7 disrupts the effects of Raf on Akt (FIG. 8), and this correlates with continued phosphorylation and reduced nuclear localization of p21^{Cip1} in Raf-activated E7 cells (FIG. 7). The ability of E7 to preserve Akt activity participates in E7's ability to overcome Raf-induced arrest, since chemical inhibition of the PI3-K/Akt pathway or introduction of a dominant-negative Akt perturbs E7-mediated cell cycle progression in the presence of Raf stimulation (FIG. 8). Indeed, expression of a constitutively active form of Akt restored G1-S progression during Raf-activation, underlining Akt as an important target in rescue from Raf-induced arrest. An LXCXE-mutated form of E7 that could not prevent the Raf-induced effects on Akt was also impaired in its capacity to abrogate Raf arrest (FIG. 9). Since the LXCXE motif is essential for the interaction between E7 and the RB tumor suppressor (Barbosa et al., 1990; Munger et al., 1989), the effects of E7 on Akt may involve dysregulation of RB-targeted genes.

Oncogenic activation of the Ras/Raf/MAPK pathway can lead to a p21^{Cip1}-dependent cell cycle arrest (Lloyd et al. Genes & Development. 11:663-77 (1997), Sewing et al. Molecular & Cellular Biology 17:5588-97 (1997), Woods et al. Molecular & Cellular Biology 17:5598-611 (1997)). HPV 16 E7 transforms primary cells in cooperation with Ras and abrogates growth arrest elicited by various antimitogenic signals that induce p21^{Cip1} expression (Demers et al. J. Virol. 70:6862-9 (1996), Phelps et al. Cell. 53:539-47 (1988), Vousden et al. Oncogene Res. 3:167-75 (1988))). Disclosed herein, HPVI6 E7 ablates the CDK-inhibitory function of p21^{Cip1} in response to Raf activation by inhibiting its nuclear accumulation. Also disclosed is that Akt, a regulator of p21^{Cip1} localization, is required for the ability of E7 to impair p21^{Cip1} nuclear accumulation and Raf-induced arrest. The ability to impinge on p21^{Cip1} function is conserved in several Ras-cooperating oncogenes. However, these oncogenes target p21^{Cip1} by differing mechanisms. For instance, SV40 Large T antigen interacts with and inactivates the p53 tumor suppressor, a transcriptional activator of p21^{Cip1} (Dyson et al. Princess Takamatsu Symposia. 20:191-8 (1989), el-Deiry et al. Cell. 75:817-25 (1993)). In rat Schwann cells, expression of Large T antigen prevents Raf-induced, p53-mediated expression of p21^{Cip1}, resulting in a mitogenic cellular response to Raf (Lloyd et al. 1997). Alternatively, other Ras-cooperating oncogenes do not affect the expression or accumulation of p21^{Cip1}. Myc activates expression of factors that sequester p21^{Cip1} and the closely related p27^{Kip1} (Perez-Roger et al. Eembo Journal. 18:5310-20 (1999), Vlach et al. EMBO Journal. 15:6595-604 (1996)). In the context of Raf-signaling, Myc restores cyclin E-CDK2 activity by inducing expression of cyclin E and cyclin D2, with cyclin D-CDK complexes sequestering p21^{Cip1} and cyclin E feeding a p21^{Cip1}-free pool of activatable cyclin E-CDK2 (Bouchard et al. EMBO Journal. 18:5321-33 (1999), Perez-Roger et al. (1990)). Similar to Myc, E7 induces synthesis of cyclin E (FIG. 2A) (Funk et al. Genes & Development 11:2090-100 (1997), Zerfass et al. J. Virol. 69:6389-99 (1995)). Increased levels of cyclin E may elevate the pool of total cyclin E-CDK2 in E7-expressing cells, but, similar to previous observations (Perez-Roger et al. Oncogene. 14:2373-2381 (1997), is insufficient to bypass Raf-induced arrest or restore cyclin E-CDK2 activity (FIG. 5A and FIG. 5B).

The results disclosed herein suggest a mechanism by which E7 prevents Raf-induced, p21^{Cip1}-mediated inhibition of cyclin E-CDK2. It is disclosed that Raf-activation enhances the nuclear localization of p21^{Cip1} (FIG. 7), and E7 expression reduces this Raf-specific p21^{Cip1} nuclear accumulation. Since p21^{Cip1} is thought to establish a regulatory threshold that must be overcome for CDK2 activation (Cai et al. PNAS USA 95:12254-9 (1998), Harper et al. Cell. 6:387-400 (1995), Hengst et al. Genes & Development. 12:3882-8 (1998)), disclosed herein a reduction in nuclear p21^{Cip1} would effectively lower the “local threshold” of p21^{Cip1} in the nuclear compartment. This is consistent with the disclosed observation that E7-expressing cells exhibited an increased pool of p21^{Cip1}-free cyclin E-CDK2 despite Raf-induced elevation in p21^{Cip1} levels (FIG. 6A and FIG. 6B).

Previously, a model was proposed in which E7 abrogates p21^{Cip1} function by directly interacting with and derepressing p21^{Cip1}-associated CDK2 complexes ( Funk, J. O., S. Waga, J. B. Harry, E. Espling, B. Stillman, and D. A. Galloway 1997. Inhibition of CDK activity and PCNA-dependent DNA replication by p21 is blocked by interaction with the HPV-16 E7 oncoprotein Genes & Development. 11:2090-100., Jones, D. L., R. M. Alani, and K. Munger 1997. The human papillomavirus E7 oncoprotein can uncouple cellular differentiation and proliferation in human keratinocytes by abrogating p21Cip1-mediated inhibition of cdk2 Genes & Development. 11:2101-11.). However, the disclosed experiments do not support such a model. Interaction between E7 and p21^{Cip1} was not observed in the context of Raf signaling (FIG. 3), and could not be detected in other cell types (data not shown) (Hickman, E. S., S. Bates, and K. H. Vousden 1997. Pertubation of the p53 response by human papillomavirus type 16 E7 Journal of Virology. 71:3710-3718., Ruesch, M., and L. A. Laimins 1997. Initiation of DNA synthesis by human papillomavirus E7 oncoproteins is resistant to p21 mediated inhibition of cyclin E-cdk2 activity Journal of Virology. 71:5570-5578.). In addition, immunodepletion experiments indicate that p21^{Cip1}-associated cyclin E-CDK2 complexes are not active in the presence of E7 (FIG. 4A), suggesting that E7 does not physically derepress p21^{Cip1}-cyclin E-CDK2 complexes. Furthermore, it was shown herein that the intrinsic sensitivity of cyclin E-CDK2 to p21^{Cip1}-mediated inhibition is not altered in E7-expressing cells (FIG. 4B). Taken together, these data imply that E7 impinges on p21^{Cip1} function via an alternative mechanism as disclosed herein.

The ability of E7 to reduce nuclear accumulation of p21^{Cip1} and prevent inhibition of cyclin E-CDK2 suggests that p21^{Cip1} localization affects its regulation of CDK activity. Indeed, mutation of the nuclear localization sequence of p21^{Cip1} reduces its growth inhibitory function (Goubin et al. Oncogene. 10:2281-7 (1995), Zhou et al. Nature Call Biology. 3:245-52 (2001)). Subcellular location is an important aspect of other CKI function. For instance, the coordinate inhibition of CDK4 and CDK2 by p15^{Ink4b} and p27^{Kip1} requires proper compartmentalization of both CKIs within the cell (Reynisdottir et al. Genes & Development. 11:492-503 (1997)). It was also shown herein that E7 affects the nuclear localization of p27^{Kip1} upon serum withdrawal (FIG. 12), indicating that the ability of E7 to abrogate the localization of Kip/Cip CKIs may be conserved in the context of other biological signals.

Akt has recently been shown to regulate p21^{Cip1} localization via phosphorylation of its nuclear localization sequence (Zhou et al. (2001)). Interestingly, E7 prevents Raf-specific reduction in Akt levels and p21^{Cip1} phosphorylation (FIG. 8D), suggesting that E7 alters localization of p21^{Cip1} by targeting Akt. In accordance, inhibition of the PI(3)K/Akt signaling pathway abrogated the effects of E7 on p21^{Cip1} localization (FIG. 8B). Further experiments are required to determine the role of Akt in E7-mediated bypass of other p21^{Cip1}-associated arrest signals. However, Akt has been implicated in diverse contexts including cell survival, proliferation, and transformation, making Akt an intriguing putative target of E7 (Coffer et al. Biochemical Journal. 335:1-13 (1998), Datta et al. Genes & Development. 13:2905-27 (1999)).

Ras transduces extracellular information through a multitude of signaling cascades. Raf and Akt, components of two “parallel” Ras signaling pathways, have been shown to interact, with Akt phosphorylating and negatively regulating Raf activity (Rommel et al. Science. 286:1738-41 (1999), Zimmermann et al. Science. 286:1741-4 (1999)). In this study, we provide evidence that Raf can functionally antagonize Akt. We demonstrate that Raf can down-regulate steady-state levels of total and active Akt (FIG. 8D). Importantly, transfection with a constitutively active Akt restored cell cycle progression during Raf signaling (FIG. 8C), indicating that loss of Akt activity may be essential in Raf-induced arrest. Consistent with this, expression of E7 maintained total and active Akt levels (FIG. 8C), and disruption of Akt activity prevented E7-mediated bypass of Raf-arrest (FIG. 8A and FIG. 8C). Interestingly, an E7 mutant deficient in the ability to bind and disrupt RB does not maintain Akt activity (FIG. 9C), indicating that RB may be involved in regulating Akt or factors that control Akt activity. Nevertheless, the observation that Raf down-regulates Akt function establishes precedent for bi-directional cross-talk between these Ras-effector pathways. The antagonism between Raf and Akt suggests that the cellular response elicited by simultaneous stimulation of Raf and Akt can be affected by the intensity and duration of the stimulation. For instance, anchorage detachment in primary fibroblasts leads to Raf-dependent anoikis (Zugasti et al. Molecular & Cellular Biology. 21:6706-17 (2001)). Activation of Raf in this context requires loss of Akt activity, and exogenously restored Akt activity can disrupt Raf activation and restore cell survival. Likewise, sustained Akt activity can prevent serum- and IGF-1-induced activation of the Raf/ERK signaling cascade, promoting myotube differentiation and hypertrophy (Rommel et al. (1999), Zimmermann (1999)). Disclosed herein, Raf activation leads to loss of Akt activity and that this may be an essential event in the cellular arrest imposed by Raf.

Activation of the Ras/Raf/MAPK pathway can stimulate transformation or cellular arrest, depending on the signaling intensity and presence or absence of cooperating oncogenic mutations (Hirakawa et al. PNAS USA. 85:1519-23 (1988), Land et al. Molecular & Cellular Biology 6:1917-25 (1986), Ruley et al., H.E. Cancer Cells. 2:258-68 (1990), Weinberg, R. A. O Cancer Research. 49:3713-21 (1989.), Woods et al. (1997)). It was shown herein that the HPV16 E7 oncoprotein abrogates growth arrest imposed by Raf activity, a signal that elicits similar growth and morphological phenotypes as Ras in primary and immortal cells (Lin et al. Genes & Development. 12:3008-19 (1998), Sewing et al. (1 997)). Consistent with these observations, E7 inhibits Ras-induced arrest and senescence and subsequently transforms primary cells in cooperation with activated Ras (Phelps et al. (1988), Vousden et al. (1988)). Raf-induced arrest is dependent on the CKI p21^{Cip1} (Sewing et al. (1997), Woods et al. (1997)), and it was demonstrated herein that E7 impairs p21^{Cip1} CDK-inhibitory function during Raf signaling. As previously mentioned, other oncogenes (e.g. Myc, SV40 Large T antigen) that cooperate with Ras in cellular transformation also impair p21^{Cip1} function (Dyson et al. (1989), Perez-Roger et al. (1999)). This convergence of function implies that inactivation of p21^{Cip1} may be an important mechanism by which oncogenic mutations alter the cellular response to the activated Ras/Raf pathway from growth arrest to proliferation. Indeed, genetic ablation of p21^{Cip1} confers a proliferative advantage in Ras-transduced fibroblasts and promotes aggressive Ras-induced epithelial tumorigenesis (Missero et al. Genes & Development. 10:3065-75 (1996), Sewing et al. (1997)). These observations suggest that p21^{Cip1} is critical in suppressing Ras-induced transformation.

### 2. Example 2 Materials and Methods

Cell culture. NIH3T3 cells stably expressing the RafAR fusion protein have been described previously (Sewing et al., 1997). This cell line and all derivatives were cultured in DMEM without phenol red (GIBCO-BRL) supplemented with 10% charcoal-stripped newborn calf serum (NCS, Hyclone). Cell lines stably expressing E7, E7.C24G, or human cyclin E were established via the recombinant amphotropic pBabe retroviral system described elsewhere (Morgenstern and Land, 1990). Upon infection, cells were selected in 2.5 tig/mL puromycin (Sigma) and used for limited generations. For all experiments, asynchronous cells were seeded at low density (5×105 cells per 15 cm dish), and subsequently treated with RafAR-inducing 1.0 μM R1881 (methyltrienolone, Dupont) or vehicle (ethanol) for the indicated times. For TGF-β experiments, Mv1Lu cells with doxycycline-inducible E7 expression, tet-E7 Mv1Lu (a kind gift from M. O'Reilly), were treated with 3 ng/mL TGF-β with or without 2 μg/mL doxycycline for 24 hours.

Cell cycle analysis. Cells were pulsed with 10 μM bromodeoxyuridine (BrdU) for 30 minutes, trypsinized, and fixed in 70% ethanol for 12 hours at 4° C. Subsequently, cells were labeled with FITC-conjugated α-BrdU (Boehringer Manheim), treated with RNase A (1 mg/mL, Sigma), and stained with propidium iodide (20 μg/mL, Sigma) following standard protocols. Data was collected and analyzed by FACSCaliber (ELITE) and Multicycle software, respectively.

Immunoblotting and immunoprecipitations (IP). Cell pellets were lysed in HLB lysis buffer (50 mM Tris pH 8.0, 150 mM NaCl, 1% Triton X-100, 1 mM DTT, and protease inhibitor cocktail (Sigma P8340)) for 30 minutes with vortexing. After centrifugation, protein content was quantitated via a standard BioRad Bradford assay. For immunoblotting, 30-50 μg of cell lysate was separated by sodium dodecyl sulfate-polyacrylamide gel electrophoresis (SDS-PAGE) and transferred onto nitrocellulose membrane (Schleicher and Schuell). After incubation with primary and secondary antibodies, antigen detection was performed using the enhanced chemiluminescence kit from NEN. The following antibodies were obtained from Santa Cruz: α-cyclin D1 (sc-450, Western), α-cyclin D2 (sc-593, Western), α-cyclin E (sc-481, IP and Western), α-human cyclin E (sc-198, IP and Western), α-CDK2 (sc-163 g, IP and Western), α-CDK4 (sc-260, IP and Western), α-p21^{Cip1} (sc-6246, Western), α-p21^{Cip1} (sc-397, IP and immunofluorescence), and a-phospho-threonine (sc-5267, Western). HPV-16 E7-specific and RB-specific antibodies were purchased from Zymed and Pharmingen, respectively. Antibodies recognizing Akt or Akt phosphorylated at serine-473 were obtained from Cell Signaling. For immunoprecipitations, cell lysates were incubated with primary antibodies for 2 hours at 4° C., and immune-complexes were collected on Protein A-agarose beads (Santa Cruz) for an additional 1 hour. The complexes were washed four times with HLB buffer and resolved by 12% SDS-PAGE for immunoblotting.

Kinase assays. After immunoprecipitation, immune-complexes were washed two additional times in kinase buffer (50 mM Tris, pH 7.5, 10 mM MgCl2, 1 mM DTT). Kinase assays were performed in kinase buffer with 30 μM ATP, 3 μCi [γ-32P]ATP, and 15 μg histone H1 (for cyclin E-CDK2 complexes) or 2 μg GST-RB c-terminus (for CDK4 complexes) per reaction for 15 minutes at 23° C. Radiolabelled substrate was resolved on 12% SDS-PAGE and quantified with a phosphorimager and ImageQuant (Molecular Dynamics) software. For in vitro p21^{Cip1} inhibition experiments, purified recombinant GST-p21^{Cip1} was incubated with 20 μg of target control or E7-expressing cell lysates for 30 minutes at 30° C. before assaying for cyclin E-associated histone H1 kinase activity as described above.

Plasmids and transfections. pBabe retroviral constructs expressing E7 and E7.C24G were generated and are described elsewhere (Nead et al., 1998). The pBabe derivative expressing human cyclin E was described elsewhere (Vlach et al., 1996). The pcDNA3 constructs encoding Akt K179M was described elsewhere (Burgering and Coffer, 1995) and myristoylated Akt (myr-Akt) (Kohn et al., 1996) was described elsewhere. The GFP-expression plasmid, pEGFPC1, is commercially available (Clontech). For transient transfections, control or E7-expressing cells were seeded on 60 mm dishes at a density of 1.5×10⁵ cells/plate. Cells were transfected 24 hours later with the indicated plasmids using LipfectAmine 2000 (Invitrogen) according to manufacturer's instructions. After a 5 hour incubation, the transfection medium was replaced with DMEM without phenol red (GIBCO-BRL) supplemented with 10% charcoal-stripped newborn calf serum (NCS, Hyclone) for an additional 6 hours. Subsequently, cells were treated with vehicle (ethanol) or R1881 for an additional 30 hours, pulsed with 10 μM BrdU during the last 10 hours of treatment, and analyzed for BrdU incorporation by immunofluorescence (see below).

Immunofluorescence. For p21^{Cip1} localization experiments, cells were plated in six-well dishes and treated with R1881 or vehicle. After treatment, cells were fixed in 3.7% paraformaldehyde, perneabilized in 0.2% Triton X-100 and 10% FBS in PBS for 10 minutes, and incubated with p21^{Cip1}-specific antibodies (1:100 dilution) or normal IgG for 12 hours at 4° C. Cells were washed three times in PBS with 10% FBS and stained with fluorophore-conjugated α-rabbit (1:200 dilution, Molecular Probes) for 30 minutes. All antibody solutions were prepared in 10% FBS in PBS. For transfection experiments, cells were pulsed with BrdU for 10 hours prior to fixation. Cells were fixed and permeabilized as described for p21^{Cip1} localization experiments, and cellular DNA was denatured by incubation with DNase I (100 U/mL, Gibco) for 1 hour at 37° C. BrdU-positive cells were stained with fluorophore-conjugated α-BrdU (Molecular Probes) for 12 hours at 4° C. Antigen staining was visualized by inverted fluorescence microscopy (Olympus CK40), and images were captured with Quality Imaging camera and software. Exposure times were kept constant for each experiment.

### 3. Example 3

a) Inactivation of Akt by c-Raf-1 is Rapid:

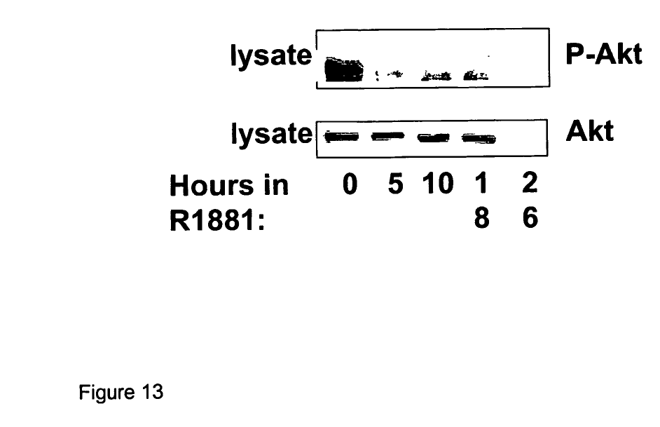

c-Raf-1 was induced by addition of the androgen analog R1881, as described herein and the cells harvested at various times thereafter. As can be seen from FIG. 13, the down-regulation of the active Akt (P-Akt) is observed within 5 hours post-treatment, while total protein levels only decrease at about 19-26 hours post-treatment. As discussed in Example 1, the cell cycle arrest occurs about 20 hours post-treatment, and is consistent with accounting for the reduced levels of non-phosphorylated-inactive Akt. Thus, while total amounts of Akt do not change at the early time points the phosphorylated active form disappears early on. The results indicate that the down-regulation of active Akt is c-raf-1 mediated and not a result of cell cycle arrest.

b) c-Raf-1 Inhibits Akt Activity Through MEK1:

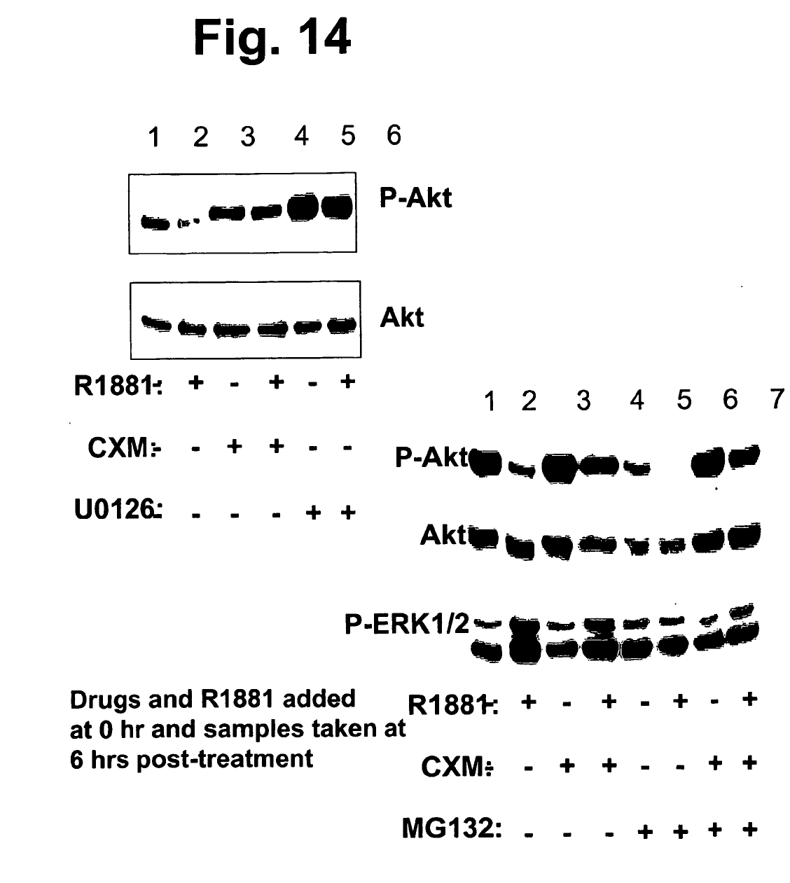

The down-regulation of activie Akt (P-Akt) on induction of c-Raf-1 requires MEK1, which is a down stream target of cRaf-1. This was determined by adding a MEK1 inhibitor (U0126) at the same time as the androgen analog, R1881 (FIG. 14, left hand panel, lanes 5 and 6). In the presence of the inhibitor active Akt down-regulation is inhibited (FIG. 14, compare lanes 1 and 2 with 5 and 6 in left panel). In fact, in the presence of the inhibitor the basal level of active Akt is increased, consistent with MEK1 normally controlling Akt activity (FIG. 14, compare lanes I and 5).

c) New Protein Synthesis is Required for c-Raf-1-Dependent Down-Regulation of Akt Activity:

To determine if new protein synthesis is required for the down-regulation of Akt, the cells were treated with cyclohexamide (a protein synthesis inhibitor, which acts on ribosomes) was added along with R1881. As can be seen from FIG. 14, (compare lanes 1 and 2 with lanes 3 and 4 in left panel), cyclohexamide treatment (CXM) inhibited the down-regulation of Akt. Therefore, new protein synthesis is required for the down-regulation of Akt by c-Raf-1.

d) Proteosome Activity Not Required for the Down-Regulation of Akt Activity:

Since there is a reduction in the active form of Akt (P-Akt) within 5 hours, it was determined if this Akt component was degraded by the proteosome. Using the proteosome inhibitor, MG132, we showed that down-regulation of P-Akt was similar to control cells (FIG. 14 compare lanes 1 and 2 with 5 and 6, right panel). Therefore, the active form of Akt is not degraded.

These results indicate that Akt is de-phosphorylated by a phosphatase protein controlled by c-Raf-1.

e) E7 Impairs DNA Damage-Induced Nuclear Localization of p21^{Cip1} in HFK.

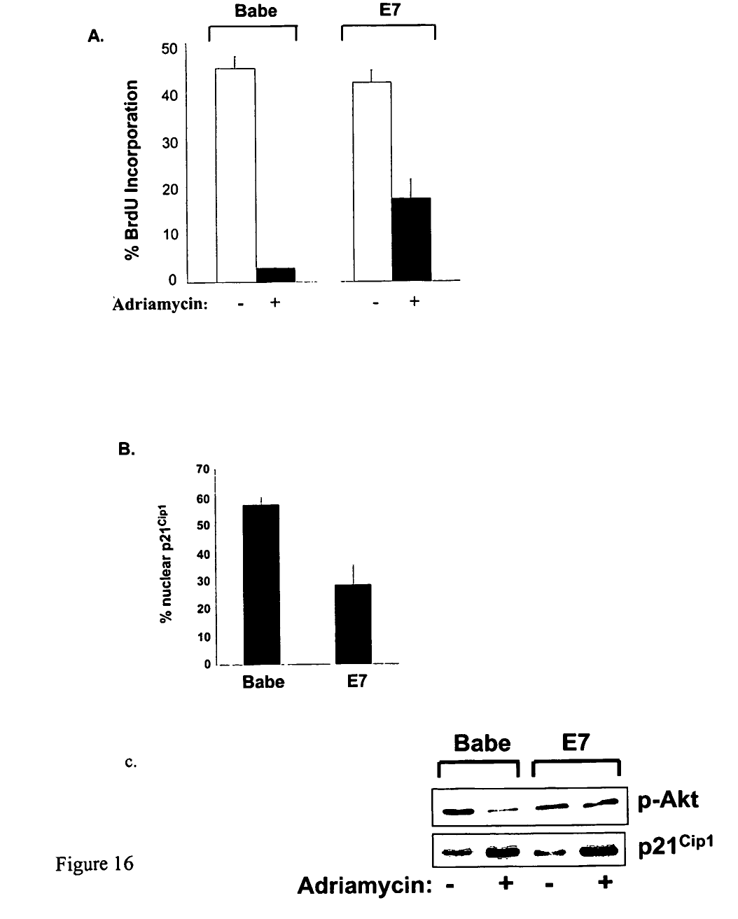

DNA damage was used to arrest cells through adriamycin treatment. Control- or E7-expressing HFK were treated with vehicle or 0.1 mM Adriamycin for 17 hours and pulsed for 1 hour prior to fixation in 4% paraformaldehyde. Subsequent to fixation, chromatin was denatured with 2N HCl, and cells were permeabilized with 0.2% Triton X-100, and stained with AlexaFluor 588-conjugated α-BrdU. Cells were scored for BrdU-positive nuclei. The number of cells with BrdU-positive nuclei is represented as a percentage of total cells counted. FIG. 16 A shows the average and standard deviation values shown are from two independent experiments with at least 300 cells counted per experiment.

In FIG. 16B cells were treated as in FIG. 16A and in addition were stained with p21^{Cip1}-specific antibody and scored for nuclear or whole-cell staining. The number of cells with nuclear p21^{Cip1} accumulation is represented as a percentage of total cells counted. FIG. 16B shows the average and standard deviation values shown are from two independent experiments with at least 200 cells counted per experiment. In FIG. 16C cell lysates were isolated at the same time as in FIG. 16A and Western blots carried out for active Akt and (FRET), in the presence of the putative inhibitor and the in absence of the putative inhibitor, wherein a decrease in FRET in the presence of the putative inhibitor as compared to FRET measurement in its absence indicates the putative inhibitor inhibits binding between the two molecules. This type of method can be performed with a cell system as well. Thus, for example, putative inhibitors could be attached to a fluorescence acceptor, and Akt could be attached to a fluorescence donor, and then in the disclosed systems, Akt activity monitored and correlated with the molecules interacting with Akt to identify molecules inhibiting Akt activity through interaction with Akt. It would also be possible to detect BrDU uptake in cells in a high throughput screen. BrDU would be taken up in cells that were not cell cycle arrested and so would indicate a drug treatment did not work.

Disclosed are cells comprising, a) a regulatable nucleic acid comprising sequence encoding a Raf gene and b) a nucleic acid comprising sequence encoding an E7 gene.

Disclosed are cells comprising, a regulatable nucleic acid comprising sequence encoding a Raf gene and sequence encoding an E7 gene.

Disclosed are cells comprising, a) a regulatable nucleic acid comprising sequence encoding a Raf gene and b) a nucleic acid comprising sequence encoding a cyclin gene.

Also disclosed are cells comprising, a regulatable nucleic acid comprising sequence encoding a Raf gene and sequence encoding a cyclin gene.

Disclosed are cells further comprising an inhibitor of Akt, an inhibitor of E7, a potential inhibitor of Akt degradation, and/or a potential inhibitor of E7 activity. Also disclosed are cells further comprising an inhibitor of PI3K.

These systems can be used to identify compositions having the desired effects on E7 and Akt described herein. For example, compositions which potentially inhibit the effect of E7 described herein can be assayed for their effect in the system, for example, those that promote p21^{Cip1} nuclear transport, through, for example, inhibiting E7 promoted maintenance of Akt. The systems can be used in a variety of ways as discussed herein.

### 4. Sequence Similarities

It is understood that as discussed herein the use of the terms homology and identity mean the same thing as similarity. Thus, for example, if the use of the word homology is used between two non-natural sequences it is understood that this is not necessarily indicating an evolutionary relationship between these two sequences, but rather is looking at the similarity or relatedness between their nucleic acid sequences. Many of the methods for determining homology between two evolutionarily related molecules are routinely applied to any two or more nucleic acids or proteins for the purpose of measuring sequence similarity regardless of whether they are evolutionarily related or not. p21^{CIP} levels before and after adriamycin treatment. FIG. 16 shows that 21^{CIP} is increased after adriamycin treatment in both control and E7-expressing HFK and that active Akt levels are maintained only in E7-expressing cells.

### F. REFERENCES

Alevizopoulos, K., Vlach, J., Hennecke, S. and Amati, B. (1997) Cyclin E and c-Myc promote cell proliferation in the presence of p161NK4a and of hypophosphorylated retinoblastoma family proteins. EMBO Journal, 16, 5322-33.

Banks, L., Edmonds, C. and Vousden, K. (1990) Ability of the HPV16 E7 protein to bind RB and induce DNA synthesis is not sufficient for efficient transforming activity in NIH3T3 cells. Oncogene, 5, 1383-1389.

Barbosa, M. S., Edmonds, C., Fisher, C., Schiller, J. T., Lowy, D. R. and Vousden, K. H. (1990) The region of the HPV E7 oncoprotein homologous to adenovirus E1a and Sv40 large T antigen contains separate domains for Rb binding and casein kinase II phosphorylation. EMBO Journal, 9, 153-60.

Bouchard, C., Thieke, K., Maier, A., Saffrich, R., Hanley-Hyde, J., Ansorge, W., Reed, S., Sicinski, P., Bartek, J. and Eilers, M. (1999) Direct induction of cyclin D2 by Myc contributes to cell cycle progression and sequestration of p27. EMBO Journal, 18, 5321-33.

Boyer, S. N., Wazer, D. E. and Band, V. (1996) E7 protein of human papilloma virus-16 induces degradation of retinoblastoma protein through the ubiquitin-proteasome pathway. Cancer Research, 56, 4620-4.

Brugarolas, J., Chandrasekaran, C., Gordon, J. I., Beach, D., Jacks, T. and Hannon, G. J. (1995) Radiation-induced cell cycle arrest compromised by p21 deficiency. Nature, 377, 552-7.

Burgering, B. M. and Coffer, P. J. (1995) Protein kinase B (c-Akt) in phosphatidylinositol-3-OH kinase signal transduction. Nature, 376, 599-602.

Cai, K. and Dynlacht, B. D. (1998) Activity and nature of p21(WAFI) complexes during the cell cycle. Proceedings of the National Academy of Sciences of the United States of America, 95, 12254-9.

Cheng, M., Olivier, P., Diehl, J. A., Fero, M., Roussel, M. F., Roberts, J. M. and Sherr, C. J. (1999) The p21 (Cip1) and p27(Kip1) CDK ‘inhibitors’ are essential activators of cyclin D-dependent kinases in murine fibroblasts. EMBO Journal, 18, 1571-83.

Coats, S., Flanagan, W. M., Nourse, J. and Roberts, J. M. (1996) Requirement of p27Kip1 for restriction point control of the fibroblast cell cycle. Science, 272, 877-80.

Coffer, P. J., Jin, J. and Woodgett, J. R. (1998) Protein kinase B (c-Akt): a multifunctional mediator of phosphatidylinositol 3-kinase activation. Biochemical Journal, 335, 1-13.

Datta, S. R., Brunet, A. and Greenberg, M. E. (1999) Cellular survival: a play in three Akts. Genes & Development, 13, 2905-27.

Davenport, E. A., Drobes, B. L., Menke, S. L., Vaidya, T. B. and Taparowsky, E. J. (1988) Cooperation of oncogenes in the multistep transformation of established fibroblasts in culture. Anticancer Research, 8, 959-69.

Demers, G. W., Espling, E., Harry, J. B., Etscheid, B. G. and Galloway, D. A. (1996) Abrogation of growth arrest signals by human papillomavirus type 16 E7 is mediated by sequences required for transformation. Journal of Virology, 70, 6862-9.

Deng, C., Zhang, P., Harper, J. W., Elledge, S. J. and Leder, P. (1995) Mice lacking p21CIP1/WAF1 undergo normal development, but are defective in G1 checkpoint control. Cell, 82, 675-84.

Dulic, V., Kaufmann, W. K., Wilson, S. J., Tlsty, T. D., Lees, E., Harper, J. W., Elledge, S. J. and Reed, S. I. (1994) p53-dependent inhibition of cyclin-dependent kinase activities in human fibroblasts during radiation-induced G1 arrest. Cell, 76, 1013-23.

Dulic, V., Stein, G. H., Far, D. F. and Reed, S. I. (1998) Nuclear accumulation of p21Cip1 at the onset of mitosis: a role at the G2/M-phase transition. Molecular & Cellular Biology, 18, 546-57.

Edmonds, C. and Vousden, K. H. (1989) A point mutational analysis of human papillomavirus type 16 E7 protein. Journal of Virology, 63, 2650-6.

el-Deiry, W. S., Tokino, T., Velculescu, V. E., Levy, D. B., Parsons, R., Trent, J. M., Lin, D., Mercer, W. E., Kinzler, K. W. and Vogelstein, B. (1993) WAF1, a potential mediator of p53 tumor suppression. Cell, 75, 817-25.

Fang, F., Orend, G., Watanabe, N., Hunter, T. and Ruoslahti, E. (1996) Dependence of cyclin E-CDK2 kinase activity on cell anchorage. Science, 271, 499-502.

Funk, J. O., Waga, S., Harry, J. B., Espling, E., Stillman, B. and Galloway, D. A. (1997) Inhibition of CDK activity and PCNA-dependent DNA replication by p21 is blocked by interaction with the HPV-16 E7 oncoprotein. Genes & Development, 11, 2090-100.

Goubin, F. and Ducommun, B. (1995) Identification of binding domains on the p21Cip1 cyclin-dependent kinase inhibitor. Oncogene, 10, 2281-7.

Halpem, A. L. a. M., K. (1997) HPV16 E7: Primary Structure and Biological Properties. Theoretical Biology and Biophysics Group, Los Alamos National Laboratory, Los Alamos.

Kohn, A. D., Takeuchi, F. and Roth, R. A. (1996) Akt, a pleckstrin homology domain containing kinase, is activated primarily by phosphorylation. Journal of Biological Chemistry, 271, 21920-6.

LaBaer, J., Garrett, M. D., Stevenson, L. F., Slingerland, J. M., Sandhu, C., Chou, H. S., Fattaey, A. and Harlow, E. (1997) New functional activities for the p21 family of CDK inhibitors. Genes & Development, 11, 847-62.

Laimins, L. A. (1993) The biology of human papillomaviruses: from warts to cancer. Infectious Agents & Disease, 2, 74-86.

Land, H., Chen, A. C., Morgenstern, J. P., Parada, L. F. and Weinberg, R. A. (1986) Behavior of myc and ras oncogenes in transformation of rat embryo fibroblasts. Molecular & Cellular Biology, 6, 1917-25.

Lloyd, A. C., Obermuller, F., Staddon, S., Barth, C. F., McMahon, M. and Land, H. (1997) Cooperating oncogenes converge to regulate cyclin/cdk complexes. Genes & Development, 11, 663-77.

Lukas, J., Herzinger, T., Hansen, K., Moroni, M. C., Resnitzky, D., Helin, K., Reed, S. I. and Bartek, J. (1997) Cyclin E induced S phase without activation of the Rb/E2F pathway. Genes & Development, 11, 1479-1492.

Martin, L. G., Demers, G. W. and Galloway, D. A. (1998) Disruption of the G1/S transition in human papillomavirus type 16 E7-expressing human cells is associated with altered regulation of cyclin E. Journal of Virology, 72, 975-85.

Missero, C., Calautti, E., Eckner, R., Chin, J., Tsai, L. H., Livingston, D. M. and Dotto, G. P. (1995) Involvement of the cell-cycle inhibitor Cip1/WAF1 and the E1A-associated p300 protein in terminal differentiation. Proceedings of the National Academy of Sciences of the United States of America, 92, 5451-5.

Missero, C., Di Cunto, F., Kiyokawa, H., Koff, A. and Dotto, G. P. (1996) The absence of p21Cip1/WAF1 alters keratinocyte growth and differentiation and promotes ras-tumor progression. Genes & Development, 10, 3065-75.

Morgan, D. O. (1995) Principles of CDK regulation. Nature, 374, 1314.

Morgenstern, J. P. and Land, H. (1990) Advanced mammalian gene transfer: high titre retroviral vectors with multiple drug selection markers and a complementary helper-free packaging cell line. Nucleic Acids Research, 18, 3587-96.

Munger, K., Werness, B. A., Dyson, N., Phelps, W. C., Harlow, E. and Howley, P. M. (1989) Complex formation of human papillomavirus E7 proteins with the retinoblastoma tumor suppressor gene product. EMBO Journal, 8, 4099-105.

Harper, J. W., Adami, G. R., Wei, N., Keyomarsi, K. and Elledge, S. J. (1993) The p21 Cdk-interacting protein Cip1 is a potent inhibitor of G1 cyclin-dependent kinases. Cell, 75, 805-16.

Harper, J. W. and Elledge, S. J. (1996) Cdk inhibitors in development and cancer. Current Opinion in Genetics & Development, 6, 56-64.

Harper, J. W., Elledge, S. J., Keyomarsi, K., Dynlacht, B., Tsai, L. H., Zhang, P., Dobrowolski, S., Bai, C., Connell-Crowley, L. and Swindell, E. (1995) Inhibition of cyclin-dependent kinases by p21. Molecular Biology of the Cell, 6, 387-400.

Hengst, L., Gopfert, U., Lashuel, H. A. and Reed, S. I. (1998) Complete inhibition of Cdk/cyclin by one molecule of p21(Cip1). Genes & Development, 12, 3882-8.

Hickman, E. S., Bates, S. and Vousden, K. H. (1997) Pertubation of the p53 response by human papillomavirus type 16 E7. Journal of Virology, 71, 3710-3718.

Hicks, G. G., Egan, S. E., Greenberg, A. H. and Mowat, M. (1991) Mutant p53 tumor suppressor alleles release ras-induced cell cycle growth arrest. Molecular & Cellular Biology, 11, 1344-52.

Hirakawa, T. and Ruley, H. E. (1988) Rescue of cells from ras oncogene-induced growth arrest by a second, complementing, oncogene. Proceedings of the National Academy of Sciences of the United States of America, 85, 1519-23.

Hofmann, F. and Livingston, D. M. (1996) Differential effects of cdk2 and cdk3 on the control of pRb and E2F function during G1 exit. Genes & Development, 10, 851-61.

Jones, D. L., Alani, R. M. and Munger, K. (1997) The human papillomavirus E7 oncoprotein can uncouple cellular differentiation and proliferation in human keratinocytes by abrogating p21Cip1-mediated inhibition of cdk2. Genes & Development, 11, 2101 -11.

Jones, D. L. and Munger, K. (1997) Analysis of the p53 -mediated G1 growth arrest pathway in cells expressing the human papillomavirus type 16 E7 oncoprotein. Journal of Virology, 71, 2905-12.

Kandel, E. S. and Hay, N. (1999) The regulation and activities of the multifunctional serine/threonine kinase Akt/PKB. Experimental Cell Research, 253, 210-29.

Kemp, C. J., Donehower, L. A., Bradley, A. and Balmain, A. (1993) Reduction of p53 gene dosage does not increase initiation or promotion but enhances malignant progression of chemically induced skin tumors. Cell, 74, 813-22.

Koff, A., Ohtsuki, M., Polyak, K., Roberts, J. M. and Massague, J. (1993) Negative regulation of G1 in mammalian cells: inhibition of cyclin E-dependent kinase by TGF-beta. Science, 260, 536-9.

Nead, M. A., Baglia, L. A., Antinore, M. J., Ludlow, J. W. and McCance, D. J. (1998) Rb binds c-Jun and activates transcription. EMBO Journal, 17, 2342-52.

Noda, A., Ning, Y., Venable, S. F., Pereira-Smith, O. M. and Smith, J. R. (1994) Cloning of senescent cell-derived inhibitors of DNA synthesis using an expression screen. Experimental Cell Research, 211, 90-8.

Ohtsubo, M., Theodoras, A. M., Schumacher, J., Roberts, J. M. and Pagano, M. (1995) Human cyclin E, a nuclear protein essential for the G1-to-S phase transition. Molecular & Cellular Biology, 15, 2612-24.

Parker, S. B., Eichele, G., Zhang, P., Rawls, A., Sands, A. T., Bradley, A., Olson, E. N., Harper, J. W. and Elledge, S. J. (1995) p53-independent expression of p21Cip1 in muscle and other terminally differentiating cells. Science, 267, 1024-7.

Perez-Roger, I., Kim, S. H., Griffiths, B., Sewing, A. and Land, H. (1999) Cyclins D1 and D2 mediate myc-induced proliferation via sequestration of p27(Kip1) and p21(Cip1). EMBO Journal, 18, 5310-20.

Perez-Roger, I., Solomon, D. L. C., Sewing, A. and Land, H. (1997) Myc activation of cyclinEcdk2 kinase involves induction of cyclin E gene transcription and inhibition of p27^{Kip1} binding to newly formed complexes. Oncogene, 14, 2373-2381.

Phelps, W. C., Munger, K., Yee, C. L., Barnes, J. A. and Howley, P. M. (1992) Structure-function analysis of the human papillomavirus type 16 E7 oncoprotein. Journal of Virology, 66, 2418-27.

Philipp, J., Vo, K., Gurley, K. E., Seidel, K. and Kemp, C. J. (1999) Tumor suppression by p27Kip1 and p21Cip1 during chemically induced skin carcinogenesis. Oncogene, 18, 4689-98.

Polyak, K., Kato, J. Y., Solomon, M. J., Sherr, C. J., Massague, J., Roberts, J. M. and Koff, A. (1994a) p27Kip1, a cyclin-Cdk inhibitor, links transforming growth factor-beta and contact inhibition to cell cycle arrest. Genes & Development, 8, 9-22.

Polyak, K., Lee, M. H., Erdjument-Bromage, H., Koff, A., Roberts, J. M., Tempst, P. and Massague, J. (1994b) Cloning of p27Kip1, a cyclin-dependent kinase inhibitor and a potential mediator of extracellular antimitogenic signals. Cell, 78, 59-66.

Quelle, D. E., Ashmun, R. A., Shurtleff, S. A., Kato, J. Y., Bar-Sagi, D., Roussel, M. F. and Sherr, C. J. (1993) Overexpression of mouse D-type cyclins accelerates G1 phase in rodent fibroblasts. Genes & Development, 7, 1559-71.

Reynisdottir, I. and Massague, J. (1997) The subcellular locations of p15(Ink4b) and p27(Kip1) coordinate their inhibitory interactions with cdk4 and cdk2. Genes & Development, 11, 492-503.

Ridley, A. J., Paterson, H. F., Noble, M. and Land, H. (1988) Ras-mediated cell cycle arrest is altered by nuclear oncogenes to induce Schwann cell transformation. EMBO Journal, 7, 1635-45.

Rommel, C., Clarke, B. A., Zimmermann, S., Nunez, L., Rossman, R., Reid, K., Moelling, K., Yancopoulos, G. D. and Glass, D. J. (1999) Differentiation stage-specific inhibition of the Raf-MEK-ERK pathway by Akt. Science, 286, 1738-41.

Roper, E., Weinberg, W., Watt, F. M. and Land, H. (2001) p19ARF-independent induction of p53 and cell cycle arrest by Raf in murine keratinocytes. EMBO Reports, 2, 145-50.

Rossig, L., Jadidi, A. S., Urbich, C., Badorff, C., Zeiher, A. M. and Dimmeler, S. (2001) Akt-dependent phosphorylation of p21(Cip1) regulates PCNA binding and proliferation of endothelial cells. Molecular & Cellular Biology, 21, 5644-57.

Rudolph, B., Saffrich, R., Zwicker, J., Henglein, B., Muller, R., Ansorge, W. and Eilers, M. (1996) Activation of cyclin-dependent kinases by Myc mediates induction of cyclin A, but not apoptosis. EMBO Journal, 15, 3065-76.

Ruesch, M. and Laimins, L. A. (1997) Initiation of DNA synthesis by human papillomavirus E7 oncoproteins is resistant to p21 mediated inhibition of cyclin E-cdk2 activity. Journal of Virology, 71, 5570-5578.

Ruesch, M. and Laimins, L. A. (1998) Human papillomavirus oncoproteins alter differentiation-dependent cell cycle exit on suspension in semisolid medium. Virology, 250, 19-29.

Schneid, M. P., Woodgett, J. R. (2001) PKB/Akt: Functional Insights from Genetic Models. Nature Reviews Molecular Cell Biology, 2, 760-768.

Schulze, A., Mannhardt, B., Zerfass-Thome, K., Zwerschke, W. and Jansen-Durr, P. (1998) Anchorage-independent transcription of the cyclin A gene induced by the E7 oncoprotein of human papillomavirus type 16. Journal of Virology, 72, 2323-34.

Serrano, M., Lin, A. W., McCurrach, M. E., Beach, D. and Lowe, S. W. (1997) Oncogenic ras provokes premature cell senescence associated with accumulation of p53 and p161NK4a. Cell, 88, 593-602.

Sewing, A., Wiseman, B., Lloyd, A. C. and Land, H. (1997) High-intensity Raf signal causes cell cycle arrest mediated by p21Cip1. Molecular & Cellular Biology, 17, 5588-97.

Sherr, C. J. (1994a) G1 phase progression: cycling on cue. Cell, 79, 551-5.

Sherr, C. J. (1994b) Growth factor-regulated G1 cyclins. Stem Cells, 12, 47-55; discussion 55-7.

Sherr, C. J. (1996) Cancer cell cycles. Science, 274, 1672-7.

Sherr, C. J. and Roberts, J. M. (1995) Inhibitors of mammalian G1 cyclin-dependent kinases. Genes & Development, 9, 1149-63.

Sherr, C. J. and Weber, J. D. (2000) The ARF/p53 pathway. Current Opinion in Genetics & Development, 10, 94-9.

Steiner, P., Philipp, A., Lukas, J., Godden-Kent, D., Pagano, M., Mittnacht, S., Bartek, J. and Eilers, M. (1995) Identification of a Myc-dependent step during the formation of active G1 cyclin-cdk complexes. EMBO Journal, 14, 4814-26.

Topley, G. I., Okuyama, R., Gonzales, J. G., Conti, C. and Dotto, G. P. (1999) p21(WAF1/Cip1) functions as a suppressor of malignant skin tumor formation and a determinant of keratinocyte stem-cell potential. Proceedings of the National Academy of Sciences of the United States of America, 96, 9089-94.

Vlach, J., Hennecke, S., Alevizopoulos, K., Conti, D. and Amati, B. (1996) Growth arrest by the cyclin-dependent kinase inhibitor p27Kip1 is abrogated by c-Myc. EMBO Journal, 15, 6595-604.

Weinberg, R. A. (1995) The retinoblastoma protein and cell cycle control. Cell, 81, 323-30.

Weinberg, W. C., Azzoli, C. G., Kadiwar, N. and Yuspa, S. H. (1994) p53 gene dosage modifies growth and malignant progression of keratinocytes expressing the v-rasHa oncogene. Cancer Research, 54, 5584-92.

Weinberg, W. C., Femandez-Salas, E., Morgan, D. L., Shalizi, A., Mirosh, E., Stanulis, E., Deng, C., Hennings, H. and Yuspa, S. H. (1999) Genetic deletion of p21WAF1 enhances papilloma formnation but not malignant conversion in experimental mouse skin carcinogenesis. Cancer Research, 59, 2050-4.

Woods, D., Parry, D., Cherwinski, H., Bosch, E., Lees, E. and McMahon, M. (1997) Raf-induced proliferation or cell cycle arrest is determined by the level of Raf activity with arrest mediated by p21Cip1. Molecular & Cellular Biology, 17, 5598-611.

Zerfass, K., Schulze, A., Spitkovsky, D., Friedman, V., Henglein, B. and Jansen-Durr, P. (1995) Sequential activation of cyclin E and cyclin A gene expression by human papillomavirus type 16 E7 through sequences necessary for transformnation. Journal of Virology, 69, 6389-99.

Zerfass-Thome, K., Schulze, A., Zwerschke, W., Vogt, B., Helin, K., Bartek, J., Henglein, B. and Jansen-Durr, P. (1997) p27KIP1 blocks cyclin E-dependent transactivation of cyclin A gene expression. Molecular & Cellular Biology, 17, 407-15.

Zhang, H., Hannon, G. J. and Beach, D. (1994a) p21-containing cyclin kinases exist in both active and inactive states. Genes & Development, 8, 1750-8.

Zhang, H., Hannon, G. J., Casso, D. and Beach, D. (1994b) p21 is a component of active cell cycle kinases. Cold Spring Harbor Symposia on Quantitative Biology, 59, 21-9.

Zhou, B. P., Liao, Y., Xia, W., Spohn, B., Lee, M. H. and Hung, M. C. (2001) Cytoplasmic localization of p21Cip1/WAF1 by Akt-induced phosphorylation in HER-2/neu-overexpressing cells. Nature Cell Biology, 3, 245-52.

Zimmermann, S. and Moelling, K. (1999) Phosphorylation and regulation of Raf by Akt (protein kinase B). Science, 286, 1741-4.

### G. SEQUENCES

1. SEQ ID NO:1 Protein hormone binding domain of human androgen receptor (aa 646-917)

2. SEQ ID NO:2 Protein c-Raf-1 (aa 305-648)

3. SEQ ID NO:3 K-ras (aa 169-188)

4. SEQ ID NO:4 human p21^{Cip1} protein sequence

5. SEQ ID NO:5 human p21^{Cip1} DNA sequence

6. SEQ ID NO:6 human Akt/PKB protein sequenc

7. SEQ ID NO:7 human Akt1 DNA sequence

8. SEQ ID NO:8 HPV16 E7 aa sequence

9. SEQ ID NO:9 HPV16 E7 DNA sequence

10. SEQ ID NO:10 HPV16 E7 degenerate DNA sequence 6^{th} nucleotide changed from T to a C at capital C, still encoding His

11. SEQ ID NO:11 HPV16 variant E7 aa sequence I to V at position 38

12. SEQ ID NO:12 DNA encoding HPV16 E7 variant set forth in SEQ ID NO:11 (A to G change at capital G)

13. SEQ ID NO:13 degenerate DNA encoding SEQ ID NO 11. T to C change at capital C still encoding His

14. SEQ ID NO:14, Accession No. AAA42001, raf protein, rattus norvegicus

15. SEQ ID NO:15, Accession No. X03484 human mRNA for raf oncogene

16. SEQ ID NO:16, Accession No. AAA28142 raf proto-oncogene, Caenorhabditis elegans

17. SEQ ID NO:17, Accession No. NM-076862 Caenorhabditis elegans MAP kinase kinase or Erk Kinase MEK-1 DNA

18. SEQ ID NO:18, Accession No. P53349, mitogen-activated protein kinase kinase kinase 1 (MAPKK1) (Erk activator kinase 1) (MAPK/ERK kinase kinase 1) (MEK1 kinase 1) (MEKK1), mus musculus

19. SEQ ID NO:19, Accession No. Q02750 Dual Specificity mitogen-activated protein kinase kinase 1 (MAP kinase kinase 1) (MAPKK1) (Erk activator kinase 1). (MAPK/ERK kinase 1) (MEK1), homo sapiens

20. SEQ ID NO:20, Accession No. P31938, Dual Specificity mitogen-activated protein kinase kinase 1 (MAP kinase kinase 1) (MAPKK1) (Erk activator kinase 1) (MAPK/ERK kinase 1) (MEK1), mus musculus

21. SEQ ID NO:21, Accession No. NP-002746 mitogen-activated protein kinase kinase 1, protein kinase, mitogen activated, kinase 1 (MAP kinase kinase 1) homo sapiens

22. P04049 RAF proto-oncogene serine/threonine-protein kinase (RAF-1)

1. A method of identifying a compound that inhibits E7 cellular proliferation activity comprising,
a) administering a compound to a system, wherein the system maintains Akt activity;
b) assaying the effect of the compound on the amount of Akt activity in the system; and
c) selecting a compound which causes a decrease in the amount of Akt activity present in the system.

2. The method of claim 1, wherein the system comprises an arrest signal.

3. The method of claim 1, wherein the arrest signal comprises an inducible Raf protein or conserved variant of the Raf protein.

4. The method of claim 3, wherein the inducible Raf protein is cRaf-1 or a conserved variant of cRaf-1.

5. The method of claim 1, wherein the step of assaying the effect of the compound comprises using an antibody for Akt.

6. A method of inhibiting E7 cellular proliferation activity comprising administering a compound, wherein the compound decreases the amount of Akt activity, wherein the compound is defined as a compound capable of being identified by administering the compound to a system, wherein the system maintains Akt activity, assaying the effect of the compound on the amount of Akt activity in the system, and selecting a compound which causes a decrease in the amount of Akt activity present in the system.

7. A method of inhibiting E7 cellular proliferation activity comprising administering a compound that decreases the amount of Akt activity.

8. A method of making a composition capable of inhibiting E7 cellular proliferation activity comprising mixing an E7 inhibiting compound with a pharmaceutically acceptable carrier, wherein the compound can be identified by administering the compound to a system, wherein the system maintains Akt activity, assaying the effect of the compound on the amount of Akt activity in the system, and selecting a compound which causes a decrease in the amount of Akt activity present in the system.

9. A method of making a compound that inhibits E7 cellular proliferation activity comprising,
a) administering a compound to a system, wherein the system causes maintenance of Akt activity,
b) assaying the effect of the compound on the amount of Akt activity in the system,
c) selecting a compound which causes a decrease in the amount of Akt activity present in the system, and d) synthesizing the compound.

10. A method of identifying a compound capable of reversing the effect E7 has on Akt comprising,
a) administering a compound to a system, wherein the system comprises E7 maintenance of Akt activity,
b) assaying the effect of the compound on E7 maintenance of Akt activity, and
c) selecting a compound which inhibits E7 maintenance of Akt activity.

11. A method of inhibiting E7 cellular proliferation activity comprising administering a compound, wherein the compound is identified as decreasing Akt activity.

12. A method of inhibiting E7 cellular proliferation activity comprising administering an inhibitor of E7 maintenance of Akt activity, wherein the inhibitor is a compound capable of being identified by administering the compound to a system, wherein the system comprises E7 maintenance of Akt activity, assaying the effect of the compound on E7 maintenance of Akt activity, and selecting a compound which inhibits E7 maintenance of Akt activity.

13. A method of inhibiting E7 cellular proliferation activity comprising administering an inhibitor of E7 maintenance of Akt activity.

14. A method of making a composition capable of inhibiting E7 maintenance of Akt activity comprising mixing the compound with a pharmaceutical carrier and wherein the compound can be identified by administering the compound to a system, wherein the system comprises E7 maintenance of Akt activity, assaying the effect of the compound on E7 maintenance of Akt activity, and selecting a compound which inhibits E7 maintenance of Akt activity.

15. A method of making a compound capable of reversing the effect E7 has on Akt comprising,
a) administering a compound to a system, wherein the system comprises E7 maintenance of Akt activity,
b) assaying the effect of the compound on E7 maintenance of Akt activity,
c) selecting a compound which inhibits E7 maintenance of Akt activity, and
d) synthesizing the compound.

16. A method of inhibiting E7 cellular proliferation activity comprising administering a compound, wherein the compound is identified as inhibiting E7 maintenance of Akt activity.

17. A method of identifying a compound which promotes the nuclear localization of p21^{Cip1} comprising,
a) administering a compound to a system, wherein the system comprises E7 p21^{Cip1} cytoplasmic localization activity,
b) assaying the effect of the compound on E7 p21^{Cip1} cytoplasmic localization activity, and
c) selecting a compound which promotes p21^{Cip1} nuclear localization activity.

18. A method of promoting p21^{Cip1} nuclear localization, comprising
a) administering a compound to a system, wherein the system comprises E7 p21^{Cip1} cytoplasmic localization activity,
b) assaying the effect of the compound on E7 p21^{Cip1} cytoplasmic localization activity, and
c) selecting a compound which promotes p21^{Cip1} nuclear localization activity.

19. A method of identifying an inhibitor of an interaction between Akt and E7 comprising
a) administering a compound to a system, wherein the system comprises E7,
b) assaying the effect of the compound on an E7-Akt interaction, and
c) selecting a compound which inhibits E7 Akt interaction.

20. A cell comprising,
a) a regulatable nucleic acid comprising sequence encoding Raf or conserved variant, and
b) a nucleic acid comprising sequence encoding an E7 or conserved variant.

21. The cell of claim 21, wherein the Raf is cRaf-1 or conserved variant.

22. A cell comprising,
a) a regulatable nucleic acid comprising sequence encoding Raf or conserved variant and sequence encoding E7 or conserved variant.

23. The cell of claim 20 further comprising an inhibitor of Akt.

24. The cell of claim 20 further comprising and inhibitor of E7.

25. The cell of claim 20 further comprising an inhibitor of PI3K.

26. A cell comprising,
a) a regulatable nucleic acid comprising sequence encoding Raf or conserved variant and
b) a nucleic acid comprising sequence encoding cyclin or conserved variant.

27. A cell comprising,
a) a regulatable nucleic acid comprising sequence encoding Raf or conserved variant and sequence encoding cyclin or conserved variant.

28. A method of inhibiting aberrant cellular proliferation comprising, administering a compound which inhibits E7 maintenance of Akt activity.

29. The method of claim 26, wherein administering the compound occurs in a subject.

30. The method of claim 27, wherein the subject is a subject who has cancer.

31. A method of inhibiting E7 cellular proliferation activity, comprising administering a compound that promotes or maintains MEK-1 activity.
